(19) United States
(12) Patent Application Publication
(10) Pub. No.: US 2023/0398265 A1
(43) Pub. Date: Dec. 14, 2023

(54) COMPOSITIONS AND METHODS FOR INHIBITION OF TISSUE ADHESION

(71) Applicant: WAYNE STATE UNIVERSITY, Detroit, MI (US)

(72) Inventors: Zhiqiang CAO, Troy, MI (US); Ershuai ZHANG, Troy, MI (US)

(21) Appl. No.: 18/252,416

(22) PCT Filed: Nov. 9, 2021
(86) PCT No.: PCT/US2021/058655

§ 371 Date: May 10, 2023

Related U.S. Application Data
(60) Provisional application No. 63/111,952, filed on Nov. 10, 2020.

(51) Int. Cl. A61L 31/06; A61K 9/00

(52) U.S. Cl. CPC A61L 31/06; A61K 9/0024; A61L 2400/12

(57) ABSTRACT

The present disclosure provides compositions and methods for inhibition of tissue adhesion. According to specific aspects, implantable anti-adhesion zwitterionic compositions which include: an aqueous liquid; and a zwitterionic polymer are disclosed, wherein the zwitterionic polymer is, consists essentially of, or includes, poly(3-((3-acrylamidopropyl)dimethylammonio)propanoate) (PCBAA). According to specific aspects, implantable anti-adhesion zwitterionic compositions are provided which include nanoparticles and/or microparticles of zwitterionic hydrogel, wherein the zwitterionic hydrogel is, consists essentially of, or includes, PCBAA. Methods of inhibiting tissue adhesion in a subject in need thereof are provided according to aspects of the present disclosure which include administering an implantable anti-adhesion zwitterionic composition to a tissue surface exposed by surgery.

### REFERENCE TO RELATED APPLICATION

This application claims priority to U.S. Provisional Patent Application Ser. No. 63/111,952, filed Nov. 10, 2020, the entire content of which is incorporated herein by reference.

### GRANT REFERENCE

This invention was made with government support under Grant No. DP2DK111910 awarded by the National Institute of Diabetes and Digestive and Kidney Diseases of the National Institutes of Health. The Government has certain rights in the invention.

### FIELD OF THE INVENTION

The present disclosure relates generally to compositions and methods for inhibition of tissue adhesion. According to specific aspects, the present disclosure relates to implantable anti-adhesion zwitterionic compositions which include: an aqueous liquid; and a zwitterionic polymer, wherein the zwitterionic polymer is, consists essentially of, or includes, poly(3-((3-acrylamidopropyl)dimethylammonio)propanoate) (PCBAA). According to specific aspects of the present disclosure, implantable anti-adhesion zwitterionic compositions are provided comprising nanoparticles and/or microparticles of zwitterionic hydrogel, wherein the zwitterionic hydrogel is, consists essentially of, or includes, PCBAA.

### BACKGROUND OF THE INVENTION

Postoperative peritoneal adhesions are frequent complications for almost any types of abdominal and pelvic surgery and are found in up to 93% of the patients. Without wishing to be bound by theory, post-operative adhesions are believed to be triggered by a mass of serosanguinous exudates on the traumatized surface within a few hours after surgery. The exudate contains platelets and extracellular matrices, and activated coagulation cascade and fibrin deposition at the wound surface—a natural wound healing process. The fibrin matrix serves as a weak, temporal adhesive and a tissue-tissue adherence can quickly form. The matrix is then invaded by inflammatory cells which further recruits other cells, in particular fibroblasts, enriched by a few days after a surgery. The temporal fibrin matrix was gradually replaced with clustered and aligned fibroblasts together with the collagen secretion, resulting in a mature and permanent adhesion.

Postoperative adhesions are severe issues leading to many adverse consequences including chronic pain, female infertility, intestinal obstruction, and even death. Typically, treatment of postoperative adhesions has required further surgical intervention (e.g. adhesiolysis). However, these established tissue adhesions from previous surgery can result in difficult surgical procedures and longer operation times during the reoperation. In addition, patients may suffer a high risk of recurrent adhesion following the surgical lysis of pre-existing adhesions.

There is an urgent need for compositions and methods to ameliorate or prevent postoperative adhesions and thereby significantly improve clinical outcomes.

### SUMMARY OF THE INVENTION

Implantable anti-adhesion zwitterionic compositions according to aspects of the present disclosure are provided which include: an aqueous liquid; and a zwitterionic polymer, wherein the zwitterionic polymer is, consists essentially of, or includes, PCBAA having the structural formula:

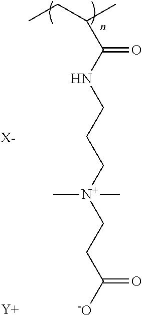

where n is an integer in the range of 2 to about 100,000, X⁻, if present, is a counter ion associated with the cationic center, and Y+, if present, is a metal ion, an ammonium ion, or an organic ion. According to aspects of the present disclosure, the zwitterionic polymer has an average molecular weight in the range of: 1 kDa to 100 kDa, 10 kDa to 100 kDa, 20 kDa to 80 kDa, or 30 kDa to 70 kDa. According to aspects of the present disclosure, the zwitterionic polymer has an average molecular weight in the range of: 1 kDa to 100 kDa, 10 kDa to 100 kDa, 20 kDa to 80 kDa, or 30 kDa to 70 kDa. According to aspects of the present disclosure, the zwitterionic polymer is present in an amount in the range of: 1 wt % to 90 wt %, 1 wt % to 60 wt %, 5 wt % to 40 wt %, 10 wt % to 40 wt %, 10 wt % to 30 wt %, or 20 wt % to 30 wt %. According to aspects of the present disclosure, the implantable anti-adhesion zwitterionic composition is biodegradable and/or pharmaceutically acceptable. According to aspects of the present disclosure, the implantable anti-adhesion zwitterionic composition is provided in a packaged dosage unit.

According to aspects of the present disclosure, implantable anti-adhesion zwitterionic compositions are provided comprising nanoparticles and/or microparticles of zwitterionic hydrogel, wherein the zwitterionic hydrogel is, consists essentially of, or includes, PCBAA. According to aspects of the present disclosure, the average particle diameter of the nanoparticles and/or microparticles of zwitterionic hydrogel is in the range of: 1 nanometer to 1000 microns, 0.01 micron to 1000 microns, 0.01 micron to 500 microns, 0.1 micron to 500 microns, 1 micron to 500 microns, 1 micron to 300 microns, 1 micron to 100 microns, 5 microns to 100 microns, 5 microns to 50 microns, 10 microns to 50 microns, 20 microns to 50 microns, 0.1 micron to 50 microns, 0.1 micron to 20 microns, 0.1 micron to 10 microns, or 0.1 micron to 1 micron. According to aspects of the present disclosure, the zwitterionic polymer has an average molecular weight in the range of: 1 kDa to 100 kDa, 10 kDa to 100 kDa, 20 kDa to 80 kDa, or 30 kDa to 70 kDa. According to aspects of the present disclosure, the zwitterionic polymer has an average molecular weight in the range of: 1 kDa to 100 kDa, 10 kDa to 100 kDa, 20 kDa to 80 kDa, or 30 kDa to 70 kDa. According to aspects of the present disclosure, the zwitterionic polymer is present in an amount in the range of: 1 wt % to 90 wt %, 1 wt % to 60 wt %, 5 wt % to 40 wt %, 10 wt % to 40 wt %, 10 wt % to 30 wt %, or 20 wt % to 30 wt %. According to aspects of the present disclosure, the nanoparticles and/or microparticles of zwitterionic hydrogel are present in an amount in the range of: 0.01 wt % to 40 wt %, 0.1 wt % to 40 wt %, 1 wt % to 30 wt %, 1 wt % to 20 wt %, 2 wt % to 10 wt %, 2 wt % to 6 wt %, relative to the aqueous liquid. According to aspects of the present disclosure, the implantable anti-adhesion zwitterionic composition is biodegradable and/or pharmaceutically acceptable. According to aspects of the present disclosure, the implantable anti-adhesion zwitterionic composition is provided in a packaged dosage unit.

Methods of inhibiting tissue adhesion in a subject in need thereof, are provide according to aspects of the present disclosure which include: administering an effective amount of an implantable anti-adhesion zwitterionic composition of the present disclosure to a tissue surface of the subject in need thereof. According to aspects of the present disclosure, the tissue surface is exposed by surgery. According to aspects of the present disclosure, the implantable anti-adhesion zwitterionic composition requires no curing following administration. According to aspects of the present disclosure, the implantable anti-adhesion zwitterionic composition undergoes no polymerization or crosslinking reaction following administration. According to aspects of the present disclosure, the implantable anti-adhesion zwitterionic composition is flowable and/or malleable, and conforms to the tissue surface. According to aspects of the present disclosure, the implantable anti-adhesion zwitterionic composition does not undergo further chemical reaction which leads to an increase of the MW of the composition following administration.

According to aspects of the present disclosure, a packaged dosage unit of an implantable anti-adhesion zwitterionic composition, comprising: an aqueous liquid and a zwitterionic polymer or copolymer, the zwitterionic polymer or copolymer comprising a plurality of repeating units, where each repeating unit has structural formula (VI), (VIII), or (X):

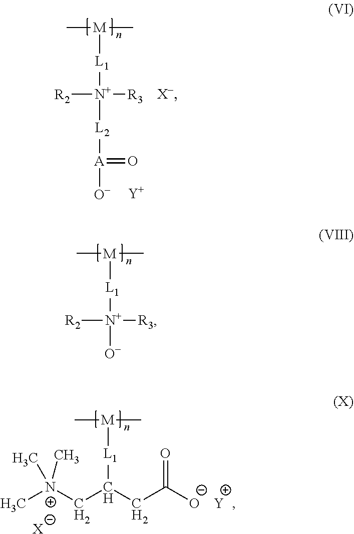

where M is a monomeric repeating unit, n is an integer from 1 to about 100,000, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion. According to aspects of the present disclosure, A is C, S, SO, P, or PO. According to aspects of the present disclosure, M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin.

According to aspects of the present disclosure, a packaged dosage unit is provided including an implantable anti-adhesion zwitterionic composition wherein the zwitterionic polymer and/or copolymer comprises a polymerization reaction product of a zwitterionic monomer having structural formula (I), (III), or (V):

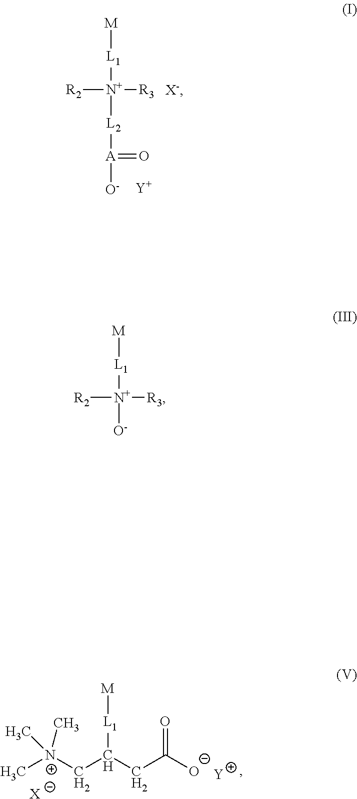

where M is a monomeric repeating unit, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion. According to aspects of the present disclosure, A is C, S, SO, P, or PO. According to aspects of the present disclosure, M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin.

According to aspects of the present disclosure, a packaged dosage unit is provided including an implantable anti-adhesion zwitterionic composition wherein the zwitterionic polymer or copolymer comprises a plurality of repeating units, where each repeating unit has structural formula:

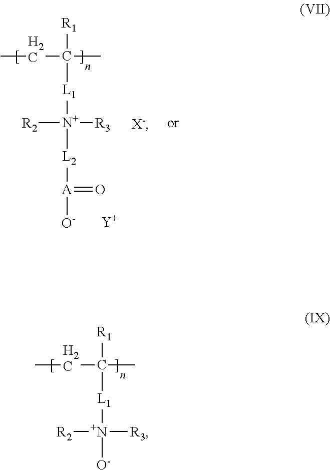

where R₁, R₂, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion. According to aspects of the present disclosure, A is C, S, SO, P, or PO. According to aspects of the present disclosure, a packaged dosage unit is provided further including a crosslinker covalently bound to at least two of the repeating units. According to aspects of the present disclosure, the crosslinker is present in an amount in the range of 0.001 wt % to 50 wt %, 0.001 wt % to 20 wt %, 0.01 wt % to 10 wt %, 0.01 wt % to 5 wt %, 0.01 wt % to 1 wt %, 0.1 wt % to 1 wt %, relative to n repeating units.

According to aspects of the present disclosure, a packaged dosage unit is provided including an implantable anti-adhesion zwitterionic composition wherein the zwitterionic polymer and/or copolymer comprises a polymerization reaction product of a zwitterionic monomer having structural formula:

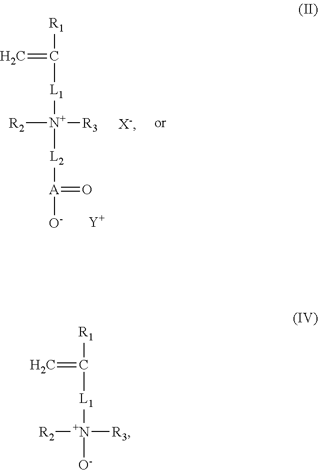

where R₁, R₂, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion. According to aspects of the present disclosure, a packaged dosage unit is provided further including a crosslinker covalently bound to at least two of the monomeric repeating units. According to aspects of the present disclosure, the crosslinker is present in an amount in the range of 0.001 wt % to 50 wt %, 0.001 wt % to 20 wt %, 0.01 wt % to 10 wt %, 0.01 wt % to 5 wt %, 0.01 wt % to 1 wt %, 0.1 wt % to 1 wt %, relative to n monomeric repeating units. According to aspects of the present disclosure, A is C, S, SO, P, or PO.

According to aspects of the present disclosure, a packaged dosage unit is provided which further includes a polymerization reaction product of a zwitterionic copolymer having reactive groups and comprising the structural formula (XI):

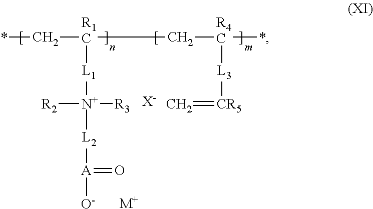

where R₁, R₂, R₃, R₄, and R₅ are each independently selected from hydrogen, alkyl, and aryl groups; L₁ is a linker that covalently couples a cationic center to the polymer backbone; L₂ is a linker that covalently couples the cationic center to an anionic group; A(═O)O⁻ is the anionic group; A is C, S, SO, P, or PO; X⁻ is the counter ion associated with the cationic center; and M⁺ is a metal ion, an ammonium ion, or an organic ion; L₃ is a linker that covalently couples a double bond to a polymer backbone, n is an integer in the range of 2 to about 100,000, m is a positive non-zero number; and m/n is in the range of 0.1%-10000%. According to aspects of the present disclosure, m/n is in the range of: 0.2% to 10%, 0.25% to 10%, 0.5% to 10%, 0.75% to 10%, 1% to 10%, 2% to 10%, 3% to 10%, 4% to 10%, 5% to 10%, 6% to 10%, 7% to 10%, 8% to 10%, 9% to 10%, 0.2% to 50%, 0.25% to 50%, 0.5% to 50%, 0.75% to 50%, 1% to 50%, 2% to 50%, 3% to 50%, 4% to 50%, 5% to 50%, 6% to 50%, 7% to 50%, 8% to 50%, 9% to 50%, 10% to 50%, 15% to 50% or 20% to 50%.

According to aspects of the present disclosure, a packaged dosage unit is provided which further includes a polymerization reaction product of a zwitterionic copolymer having reactive groups and comprising the structural formula (XII):

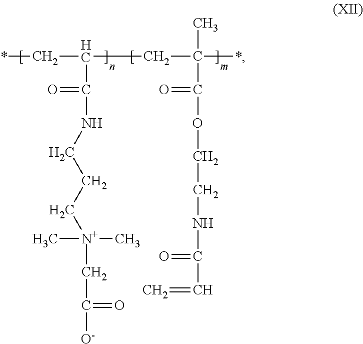

where n is an integer in the range of 2 to about 100,000, m is a positive non-zero number; and m/n is in the range of 0.1% to 10000%. According to aspects of the present disclosure, m/n is in the range of: 0.2% to 10%, 0.25% to 10%, 0.5% to 10%, 0.75% to 10%, 1% to 10%, 2% to 10%, 3% to 10%, 4% to 10%, 5% to 10%, 6% to 10%, 7% to 10%, 8% to 10%, 9% to 10%, 0.2% to 50%, 0.25% to 50%, 0.5% to 50%, 0.75% to 50%, 1% to 50%, 2% to 50%, 3% to 50%, 4% to 50%, 5% to 50%, 6% to 50%, 7% to 50%, 8% to 50%, 9% to 50%, 10% to 50%, 15% to 50% or 20% to 50%.

According to aspects of the present disclosure, a packaged dosage unit is provided including an implantable anti-adhesion zwitterionic composition comprising nanoparticles and/or microparticles of zwitterionic hydrogel.

According to aspects of the present disclosure, a packaged dosage unit is provided including an implantable anti-adhesion zwitterionic composition comprising nanoparticles and/or microparticles of zwitterionic hydrogel, wherein the average particle diameter of the nanoparticles and/or microparticles of zwitterionic hydrogel is in the range of: 1 nanometer to 1000 microns, 0.01 micron to 1000 microns, 0.01 micron to 500 microns, 0.1 micron to 500 microns, 1 micron to 500 microns, 1 micron to 300 microns, 1 micron to 100 microns, 5 microns to 100 microns, 5 microns to 50 microns, 10 microns to 50 microns, 20 microns to 50 microns, 0.1 micron to 50 microns, 0.1 micron to 20 microns, 0.1 micron to 10 microns, or 0.1 micron to 1 micron.

According to aspects of the present disclosure, a packaged dosage unit is provided including an implantable anti-adhesion zwitterionic composition comprising the zwitterionic polymer and/or copolymer, wherein the zwitterionic polymer and/or copolymer has an average molecular weight in the range of: 1 kDa to 100 kDa, 10 kDa to 100 kDa, 20 kDa to 80 kDa, or 30 kDa to 70 kDa.

According to aspects of the present disclosure, a packaged dosage unit is provided including an implantable anti-adhesion zwitterionic composition comprising the zwitterionic polymer and/or copolymer, wherein the zwitterionic polymer and/or copolymer is present in an amount in the range of: 1 wt % to 90 wt %, 1 wt % to 60 wt %, 5 wt % to 40 wt %, 10 wt % to 40 wt %, 10 wt % to 30 wt %, or 20 wt % to 30 wt %.

According to aspects of the present disclosure, a packaged dosage unit is provided including an implantable anti-adhesion zwitterionic composition comprising nanoparticles and/or microparticles of zwitterionic hydrogel, wherein the nanoparticles and/or microparticles of zwitterionic hydrogel are present in an amount in the range of: 0.01 wt % to 40 wt %, 0.1 wt % to 40 wt %, 1 wt % to 30 wt %, 1 wt % to 20 wt %, 2 wt % to 10 wt %, 2 wt % to 6 wt %, relative to the aqueous liquid.

According to aspects of the present disclosure, a packaged dosage unit is provided including an implantable anti-adhesion zwitterionic composition, wherein the implantable anti-adhesion zwitterionic composition is biodegradable and/or pharmaceutically acceptable.

According to aspects of the present disclosure, a packaged dosage unit is provided including an implantable anti-adhesion zwitterionic composition, wherein the zwitterionic polymer is, consists essentially of, or comprises, PCBAA having the structural formula:

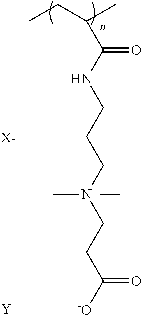

where n is an integer in the range of 2 to about 100,000, X⁻, if present, is a counter ion associated with the cationic center, and Y+, if present, is a metal ion, an ammonium ion, or an organic ion.

Implantable anti-adhesion zwitterionic compositions are provided according to aspects of the present disclosure which include: an aqueous liquid and a zwitterionic polymer or copolymer, the zwitterionic polymer or copolymer comprising a plurality of repeating units, where each repeating unit has structural formula (VI), (VIII), or (X):

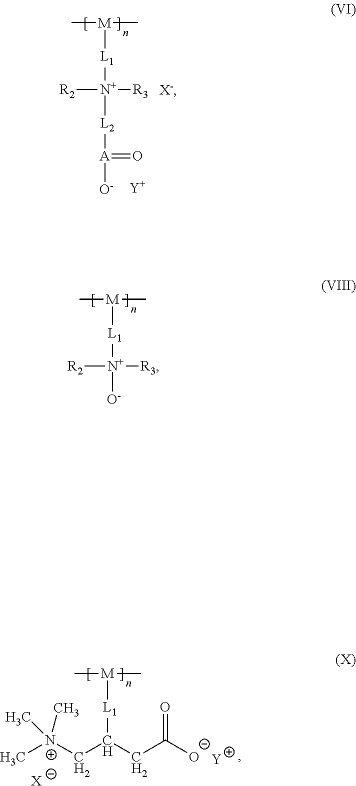

where M is a monomeric repeating unit, n is an integer from 1 to about 100,000, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion. According to aspects of the present disclosure, M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin.

Implantable anti-adhesion zwitterionic compositions are provided according to aspects of the present disclosure which include: an aqueous liquid and a zwitterionic polymer or copolymer, wherein the zwitterionic polymer and/or copolymer comprises a polymerization reaction product of a zwitterionic monomer having structural formula (I), (III), or (V):

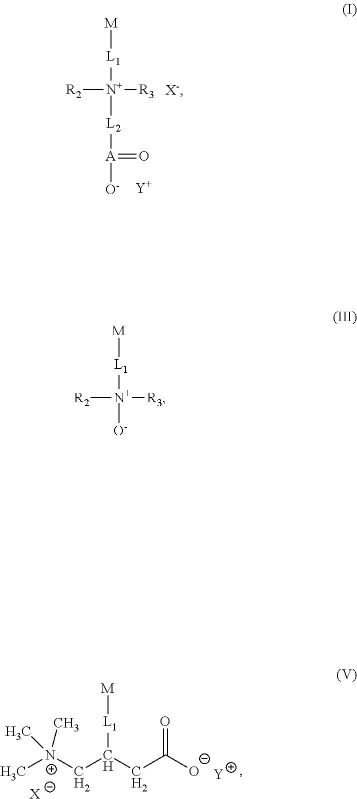

where M is a monomeric repeating unit, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion. According to aspects of the present disclosure, M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin.

Implantable anti-adhesion zwitterionic compositions are provided according to aspects of the present disclosure which include: an aqueous liquid and a zwitterionic polymer or copolymer, wherein the zwitterionic polymer and/or copolymer comprises a plurality of repeating units, where each repeating unit has structural formula:

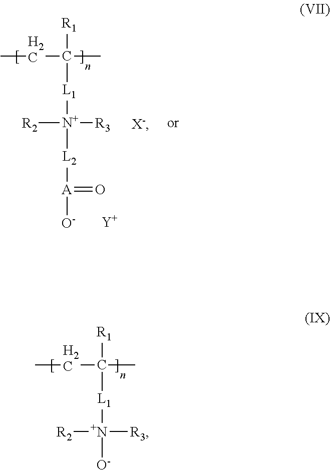

where R₁, R₂, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

Implantable anti-adhesion zwitterionic compositions are provided according to aspects of the present disclosure which include: an aqueous liquid and a zwitterionic polymer or copolymer, wherein the zwitterionic polymer and/or copolymer comprises a polymerization reaction product of a zwitterionic monomer having structural formula:

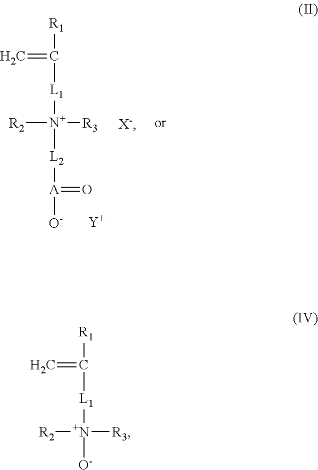

where R₁, R₂, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

Implantable anti-adhesion zwitterionic compositions are provided according to aspects of the present disclosure which include: an aqueous liquid and a zwitterionic polymer or copolymer, and further include a crosslinker covalently bound to at least two of the monomeric repeating units. According to aspects of the present disclosure, the crosslinker is present in an amount in the range of 0.001 wt % to 50 wt %, 0.001 wt % to 20 wt %, 0.01 wt % to 10 wt %, 0.01 wt % to 5 wt %, 0.01 wt % to 1 wt %, 0.1 wt % to 1 wt %, relative to n monomeric repeating units

Implantable anti-adhesion zwitterionic compositions are provided according to aspects of the present disclosure which include: an aqueous liquid and a zwitterionic polymer or copolymer, and further include a polymerization reaction product of a zwitterionic copolymer having reactive groups and comprising the structural formula (XI):

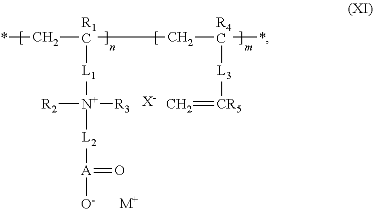

where R₁, R₂, R₃, R₄, and R₅ are each independently selected from hydrogen, alkyl, and aryl groups; L₁ is a linker that covalently couples a cationic center to the polymer backbone; L₂ is a linker that covalently couples the cationic center to an anionic group; A(═O)O⁻ is the anionic group; A is C, S, SO, P, or PO; X⁻ is the counter ion associated with the cationic center; and M⁺ is a metal ion, an ammonium ion, or an organic ion; L₃ is a linker that covalently couples a double bond to a polymer backbone, n is an integer in the range of 2 to about 100,000, m is a positive non-zero number; and m/n is in the range of 0.1%-10000%.

According to aspects of the present disclosure, A is C, S, SO, P, or PO.

Implantable anti-adhesion zwitterionic compositions are provided according to aspects of the present disclosure which include: an aqueous liquid and a zwitterionic polymer or copolymer, and further include a polymerization reaction product of a zwitterionic copolymer having reactive groups and comprising the structural formula (XII):

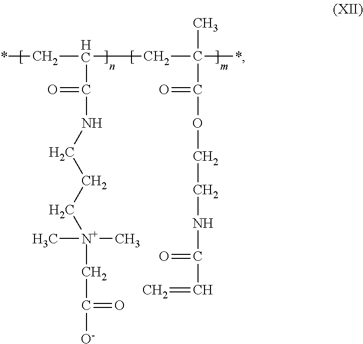

where n is an integer in the range of 2 to about 100,000, m is a positive non-zero number; and m/n is in the range of 0.1% to 10000%.

According to aspects of the present disclosure, m/n is in the range of: 0.2% to 10%, 0.25% to 10%, 0.5% to 10%, 0.75% to 10%, 1% to 10%, 2% to 10%, 3% to 10%, 4% to 10%, 5% to 10%, 6% to 10%, 7% to 10%, 8% to 10%, 9% to 10%, 0.2% to 50%, 0.25% to 50%, 0.5% to 50%, 0.75% to 50%, 1% to 50%, 2% to 50%, 3% to 50%, 4% to 50%, 5% to 50%, 6% to 50%, 7% to 50%, 8% to 50%, 9% to 50%, 10% to 50%, 15% to 50% or 20% to 50%.

Implantable anti-adhesion zwitterionic compositions are provided according to aspects of the present disclosure which include: an aqueous liquid and a zwitterionic polymer or copolymer, and/or nanoparticles and/or microparticles of a zwitterionic hydrogel, wherein the average particle diameter of the nanoparticles and/or microparticles of zwitterionic hydrogel is in the range of: 1 nanometer to 1000 microns, 0.01 micron to 1000 microns, 0.01 micron to 500 microns, 0.1 micron to 500 microns, 1 micron to 500 microns, 1 micron to 300 microns, 1 micron to 100 microns, 5 microns to 100 microns, 5 microns to 50 microns, 10 microns to 50 microns, 20 microns to 50 microns, 0.1 micron to 50 microns, 0.1 micron to 20 microns, 0.1 micron to 10 microns, or 0.1 micron to 1 micron.

According to aspects of the present disclosure, the zwitterionic polymer and/or copolymer has an average molecular weight in the range of: 1 kDa to 100 kDa, 10 kDa to 100 kDa, 20 kDa to 80 kDa, or 30 kDa to 70 kDa.

According to aspects of the present disclosure, the zwitterionic polymer and/or copolymer is present in an amount in the range of: 1 wt % to 90 wt %, 1 wt % to 60 wt %, 5 wt % to 40 wt %, 10 wt % to 40 wt %, 10 wt % to 30 wt %, or 20 wt % to 30 wt %.

Implantable anti-adhesion zwitterionic compositions are provided according to aspects of the present disclosure which include: an aqueous liquid and a zwitterionic polymer or copolymer, and/or nanoparticles and/or microparticles of a zwitterionic hydrogel, wherein the nanoparticles and/or microparticles of zwitterionic hydrogel are present in an amount in the range of: 0.01 wt % to 40 wt %, 0.1 wt % to 40 wt %, 1 wt % to 30 wt %, 1 wt % to 20 wt %, 2 wt % to 10 wt %, 2 wt % to 6 wt %, relative to the aqueous liquid.

According to aspects of the present disclosure, the implantable anti-adhesion zwitterionic composition is biodegradable and/or pharmaceutically acceptable.

Implantable anti-adhesion zwitterionic compositions are provided according to aspects of the present disclosure which include: an aqueous liquid and a zwitterionic polymer or copolymer, wherein the zwitterionic polymer is, consists essentially of, or comprises, PCBAA having the structural formula:

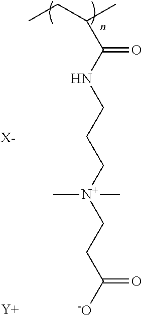

where n is an integer in the range of 2 to about 100,000, X⁻, if present, is a counter ion associated with the cationic center, and Y+, if present, is a metal ion, an ammonium ion, or an organic ion.

Methods of inhibiting tissue adhesion in a subject in need thereof are provided according to aspects of the present disclosure which include administering an effective amount of an implantable anti-adhesion zwitterionic composition of the present disclosure to a tissue surface of the subject in need thereof.

According to aspects of the present disclosure, the tissue surface is exposed by surgery.

According to aspects of the present disclosure, the implantable anti-adhesion zwitterionic composition requires no curing following administration.

According to aspects of the present disclosure, the implantable anti-adhesion zwitterionic composition undergoes no polymerization or crosslinking reaction following administration.

According to aspects of the present disclosure, the implantable anti-adhesion zwitterionic composition is flowable and/or malleable, and conforms to the tissue surface.

According to aspects of the present disclosure, the implantable anti-adhesion zwitterionic composition does not undergo further chemical reaction which leads to an increase of the MW of the composition following administration to the subject.

### BRIEF DESCRIPTION OF THE DRAWINGS

FIG. 1A is a graph showing a gel permeation chromatography (GPC) spectrum of the prepared PCBAA;

FIG. 1B is a graph showing steady-shear rheology of PCBAA solutions at 25° C. showing a shear-thinning behavior;

FIG. 1C is a graph showing in-vitro dissolution studies of PCBAA solutions with different concentrations; data presented as mean±SD (n=3);

FIG. 2 is a graph showing quantification of FITC-Fn fluorescent intensity in the region of abdominal wall defects where FITC-Fn was adsorbed onto the untreated wounds while no adsorption on the PCBAA treated defects was observed, at 2 h and 24 h after surgery; the results were presented as mean±SD (n=3); a two-tailed t-test analysis was used for statistical analysis; **P<0.01. ***P<0.001;

FIG. 3 is a graph showing quantification of fluorescent intensity in the region of abdominal wall defects wherein no significant red fluorescence-labeled fibroblasts adhesion on the PCBAA treated abdominal wall defects was observed at 2 hours, 1 day, and 4 day after the surgery while abundant fibroblasts adhered onto the untreated wounds at 1 day and 4 day after surgery; the results were presented as mean±SD (n=3); a two-tailed t-test analysis was used for statistical analysis; **P<0.01. NS, not significant;

FIGS. 4A-4E illustrate evaluation of anti-adhesion efficacy at day 7 and day 14 after surgery in a rat sidewall defect-cecum abrasion adhesion model;

FIG. 4A is a set of images showing post-operative adhesions were observed in the untreated control and Interceed® film groups while no adhesion was observed in rats treated with PCBAA on day 7 and 14 after the surgery;

FIG. 4B is a graph showing distribution of adhesion scores in the untreated, film, and PCBAA groups on day 7 and 14 after the surgery; the results are presented as mean±SD (n=6); **P<0.01; NS, not significant;

FIG. 4C is a graph showing weight loss in the untreated, film, and PCBAA groups on day 7 and 14 after the surgery; the results are presented as mean±SD (n=6); **P<0.01; NS, not significant;

FIG. 4D shows representative histology images of tissues from different groups stained with H&E (hematoxylin and eosin) on day 7 after surgery, AW: abdominal wall; CE: cecal mucosa; Me: mesothelial layer; SK: skeletal muscle of AW, scale bar, 400 μm;

FIG. 4E shows representative histology images of tissues from different groups stained with Masson trichrome on day 14 after surgery, AW: abdominal wall; CE: cecal mucosa; Me: mesothelial layer; SK: skeletal muscle of AW; the deposited collagen in the adhesion site was stained in blue while muscle showed red in Masson trichrome staining, scale bar, 400 μm;

FIG. 5 is a graph showing quantification of Cy7 fluorescent intensity in region of abdominal wall defects, indicative of retained PCBAA-cy7 polymer at the application site, at 1 day, 3 days and 7 days after treatment in the rat sidewall defect-cecum abrasion adhesion model; the results are presented as mean±SD (n=3); a one-way ANOVA with Tukey multi-comparison was used for statistical analysis; *P<0.05; **P<0.01; ***P<0.001;

FIG. 6A shows histological images of adhesion tissues stained with H&E from untreated control group on day 7 and 14 after surgery in a rat sidewall defect-cecum abrasion model; scale bar, 100 μm;

FIG. 6B shows histological images of adhesion tissues stained with Masson trichrome from Interceed® film-treated group on day 7 and 14 after surgery in a rat sidewall defect-cecum abrasion model; scale bar, 100 μm;

FIG. 7 is a set of histology images of the normal abdominal wall and cecum, Me: mesothelial layer; SK: skeletal muscle; CE: cecal mucosa; SM: visceral smooth muscle, scale bar, 100 μm;

FIGS. 8A-8D show aspects of evaluation of recurrent adhesion prevention on day 7 and 14 after the second surgery in a rat repeated-injury model;

FIG. 8A is a set of images showing that severe adhesions were observed in the control and Interceed® film groups while no adhesion was observed in rats treated with PCBAA on day 7 and 14 after the second surgery;

FIG. 8B is a graph showing distribution of adhesion scores in the untreated control, film, and PCBAA groups on days 7 and 14 after the second surgery; the results are presented as mean±SD (n=6); **P<0.01; NS, not significant;

FIG. 8C is a graph showing weight loss (as percentage of starting body mass) in the untreated control, film, and PCBAA groups on days 7 and 14 after the second surgery; the results are presented as mean±SD (n=6); **P<0.01; NS, not significant;

FIG. 8D shows representative histology images of tissues stained with H&E or Masson trichrome from the indicated different treatment groups on day 7 and 14 after the second surgery, respectively; AW: abdominal wall; CE: cecal mucosa; Me: mesothelial layer; SK: skeletal muscle of AW; the deposited collagen in the adhesion site was stained in blue while muscle showed red in Masson trichrome staining; scale bar, 400 μm;

FIG. 9A shows histological images of adhesion tissues from the untreated control group stained with H&E or Masson trichrome on day 7 and 14 after the second surgery in a rat repeated-injury model; scale bar, 100 μm;

FIG. 9B shows histological images of adhesion tissues from the Interceed® film-treated group stained with H&E or Masson trichrome on day 7 and 14 after the second surgery in a rat repeated-injury model; scale bar, 100 μm;

FIGS. 10A-10E show results of evaluation of postoperative adhesions on day 7, 14 and 30 in a rat 70% hepatectomy model;

FIG. 10A is a set of images showing that severe adhesions were observed in the control and Interceed® film groups while almost no adhesion was observed in rats treated with PCBAA after the hepatectomy;

FIG. 10B is a graph showing results of scoring adhesions presented at the cut surface for rats of the control, film, and PCBAA groups after the hepatectomy; the results are presented as mean±SD (n=6); *P<0.05; **P<0.01;

FIG. 10C is a graph showing results of scoring adhesions presented in the diaphragm of rats of the control, film, and PCBAA groups after the hepatectomy; the results are presented as mean±SD (n=6); *P<0.05; **P<0.01;

FIG. 10D is a graph showing results of scoring adhesions presented in the hepatic hilum of rats of the control, film, and PCBAA groups after the hepatectomy; the results are presented as mean±SD (n=6); *P<0.05; **P<0.01;

FIG. 10E is a graph showing results of scoring adhesions presented in the remnant liver surface of rats of the control, film, and PCBAA groups after the hepatectomy; the results are presented as mean±SD (n=6); *P<0.05; **P<0.01;

FIG. 11A shows histological images of H&E stained liver from animals of the PCBAA treated group on day 7 and 14 in a rat 70% hepatectomy-induced adhesion model;

FIG. 11B shows histological images of H&E stained liver from a healthy rat;

FIGS. 12A-12C demonstrate rheological properties, biodegradability, cytotoxicity and protein/cell resistance of the zwitterionic polymer cream gels;

FIG. 12A is a graph showing frequency-dependent, under 1% strain, 25° C., oscillatory sweeps of zwitterionic polymer cream gel formulations with different crosslinker content, 0.1% or 1% BAC;

FIG. 12B is a graph showing strain-dependent, 10 rad/s frequency, 25° C., oscillatory sweeps of zwitterionic polymer cream gel formulations with different crosslinker content, 0.1% or 1% BAC;

FIG. 12C is a graph showing in-vitro biodegradation of zwitterionic polymer cream gel formulations with different crosslinker content, 0.1% or 1% BAC; data presented as mean±s.d. (n=3);

FIGS. 12D-12H demonstrate aspects of rheological properties, biodegradability, cytotoxicity and protein/cell resistance of the cream gels according to aspects of the present disclosure.

FIG. 12D is a graph showing results of cell viability tests in which NIH/3T3 fibroblast cells were incubated with either the cream gel extract and the degradation products (zwitterionic polymer cream gel degraded in DMEM with 20 mM GSH for 48 h at 37° C.) with different crosslinker content (0.1% or 1% BAC) for 24 hours; data presented as mean±s.d. n=6);

FIG. 12E is a graph showing results of live/dead imaging tests in which NIH/3T3 fibroblast cells were incubated with either the cream gel extract and the degradation products (zwitterionic polymer cream gel degraded in DMEM with 20 mM GSH for 48 h at 37° C.) with different crosslinker content (0.1% or 1% BAC) for 24 hours; scale bar=400 μm;

FIG. 12F is a graph showing fibronectin adsorption on the surface of disk gels or cream gels using a BCA protein quantification assay; data presented as mean±s.d. (n=6);

FIG. 12G is a graph showing results of quantification of NIH/3T3 fibroblasts adhered on the disk gel surface; data presented as mean±s.d. n=6;

FIG. 12H shows representative fluorescence images of NIH/3T3 fibroblasts adhered on the surfaces of tissue culture plate (TCP) or disk gel, scale bar=400 μm;

FIG. 13 shows representative fluorescent images of NIH/3T3 fibroblasts adhered on the surfaces of tissue culture plates (TCPS) or disk gels with different crosslinker content (0.1% or 1% BAC); scale bar=400 μm;

FIGS. 14A-14C show results of evaluation of anti-adhesion efficacy in a rat sidewall defect-cecum abrasion adhesion model;

FIG. 14A is a set of images showing the establishment of a rat sidewall defect-cecum abrasion adhesion model and the application of commercial Interceed® film or zwitterionic polymer cream gel onto the traumatized sites (day 0); on day 7 and day 14 post-surgery, adhesions were observed in the untreated control and Interceed® film groups while no adhesion was observed in rats treated with zwitterionic polymer cream gel;

FIG. 14B is a graph showing distribution of adhesion scores in the indicated different treatment groups on day 7 and day 14 after the surgery; data presented as mean s.d. (n=6);**P<0.01; NS, not significant;

FIG. 14C is a graph showing weight loss in the indicated different treatment groups on day 7 and day 14 after the surgery; data presented as mean±s.d. (n=6);**P<0.01; NS, not significant;

FIGS. 14D and 14E are images showing results of evaluation of anti-adhesion efficacy in a rat sidewall defect-cecum abrasion adhesion model;

FIG. 14D shows representative histology images of tissues stained with H&E or Masson trichrome from different groups on day 7 after surgery, respectively; AW: abdominal wall; CE: cecal mucosa; Me: mesothelial layer; SK: skeletal muscle of AW;

FIG. 14E shows representative histology images of tissues stained with H&E or Masson trichrome from different groups on day 14 after surgery, respectively; AW: abdominal wall; CE: cecal mucosa; Me: mesothelial layer; SK: skeletal muscle of AW;

FIG. 15 is a set of images showing results of histological examinations of major organs, heart, liver, spleen, lung, and kidney, from healthy rat and the rats treated with the zwitterionic polymer cream gel on day 7 and 14 post-surgery;

FIGS. 16A-16G show results of evaluation of anti-adhesion efficacy of compositions according to aspects of the present disclosure in a rat repeated-injury adhesion model;

FIG. 16A is a schematic illustration of the procedure schedule;

FIG. 16B is a set of images showing procedures to establish the rat repeated-injury model and apply anti-adhesion materials onto the re-injured sites; a first abdominal wall and cecum injury was created without any anti-adhesion treatment (day −7); one week later, the developed primary adhesion was separated by adhesiolysis and a second injury was created (day 0); this was immediately followed by zwitterionic polymer cream gel treatment or film treatment;

FIG. 16C is a graph showing distribution of adhesion scores; data presented as mean±s.d., n=6, **P<0.01;

FIG. 16D is a set of representative images of recurrent adhesions in different groups on day 7 and day 14 after the second surgery;

FIG. 16E is a graph showing weight loss, as a percentage of starting body mass, in different groups on day 7 and day 14 after the second surgery; data presented as mean±s.d. (n=6), NS, not significant;

FIG. 16F is a set of representative histology images of tissues from the indicated different treatment groups on day 7 after the second surgery; AW: abdominal wall; CE: cecal mucosa; Me: mesothelial layer; SK: skeletal muscle of AW;

FIG. 16G is a set of representative histology images of tissues from the indicated different treatment groups on day 14 after the second surgery; AW: abdominal wall; CE: cecal mucosa; Me: mesothelial layer; SK: skeletal muscle of AW;

FIGS. 17A-17H are images showing anti-adhesion study in a pig sidewall defect-cecum abrasion adhesion model (n=1) including the establishment of a pig sidewall defect-cecum abrasion adhesion model and the application of zwitterionic polymer PCBAA hydrogel onto the defects;

FIG. 17A is an image showing the establishment of abdominal sidewall defects;

FIG. 17B is an image showing the establishment of cecum defects;

FIG. 17C is an image showing that the peritoneal cavity in the untreated control group was closed without any anti-adhesion material applied.

FIG. 17D is an image showing postoperative adhesions on day 30 after the surgery were observed in the untreated pig;

FIG. 17E is an image showing the establishment of abdominal sidewall defects;

FIG. 17F is an image showing the establishment of cecum defects;

FIG. 17G is an image showing zwitterionic polymer PCBAA hydrogel was applied on the injured abdominal wall and cecum, followed by closure; and

FIG. 17H is an image showing postoperative adhesions on day 30 after the surgery were observed in the pig treated with PCBAA.

### DETAILED DESCRIPTION OF THE INVENTION

Scientific and technical terms used herein are intended to have the meanings commonly understood by those of ordinary skill in the art. Such terms are found defined and used in context in various standard references illustratively including Remington: The Science and Practice of Pharmacy, Lippincott Williams & Wilkins, 21st Ed., 2005; L.V. Allen, Jr. et al., Ansel's Pharmaceutical Dosage Forms and Drug Delivery Systems, 8th Ed., Philadelphia, PA: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

A packaged dosage unit of an implantable anti-adhesion zwitterionic composition is provided according to aspects of the present disclosure, including: an aqueous liquid and a zwitterionic polymer or copolymer, the zwitterionic polymer or copolymer comprising a plurality of repeating units, where each repeating unit has structural formula (VI), (VIII), or (X):

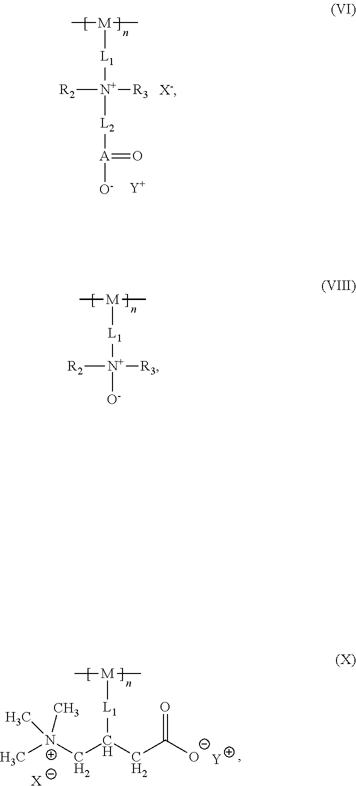

where M is a monomeric repeating unit, n is an integer from 1 to about 100,000, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

The term “packaged dosage unit” as used herein describes a closed or closable container and a dosage unit of an implantable anti-adhesion zwitterionic composition inside the container. An implantable anti-adhesion zwitterionic composition is packaged in dosage unit form for ease of administration. Dosage unit form refers to a physically discrete units of a predetermined quantity to produce an anti-adhesion effect in the subject. A container for a packaged dosage unit can be, without limitation, a syringe, a blister pack, ajar, a vial, or a bottle.

The aqueous liquid can be any pharmaceutically acceptable aqueous liquid, such as, but not limited to, water, a buffered aqueous liquid, such as phosphate buffered saline.

The term “implantable” refers to a characteristic of the anti-adhesion zwitterionic composition, being biocompatible, inserted into a subject's body, and imparting a beneficial anti-adhesion property when present in contact with one or more body tissues in a subject's body.

The term “biodegradable” refers to a characteristic of the anti-adhesion zwitterionic composition, being broken down by physiological actions of a subject's body over time when inserted into the subject's body.

According to aspects of the present disclosure, a packaged dosage unit of an implantable anti-adhesion zwitterionic composition is provided wherein the zwitterionic polymer and/or copolymer comprises a polymerization reaction product of a zwitterionic monomer having structural formula (I), (III), or (V):

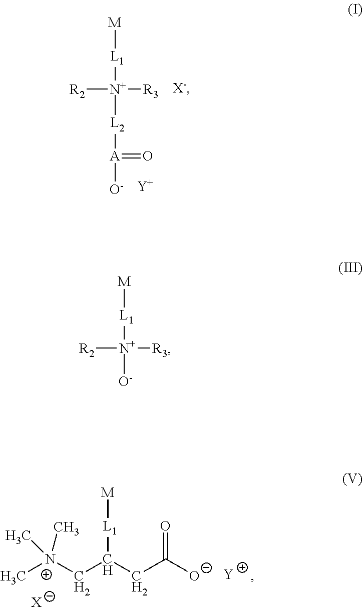

where M is a monomeric repeating unit, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

According to aspects of the present disclosure, a packaged dosage unit of an implantable anti-adhesion zwitterionic composition is provided wherein the zwitterionic polymer or copolymer comprises a plurality of repeating units, where each repeating unit has structural formula:

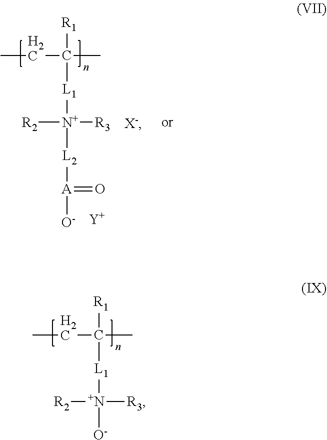

where R₁, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

According to aspects of the present disclosure, a packaged dosage unit of an implantable anti-adhesion zwitterionic composition is provided wherein the zwitterionic polymer and/or copolymer comprises a polymerization reaction product of a zwitterionic monomer having structural formula:

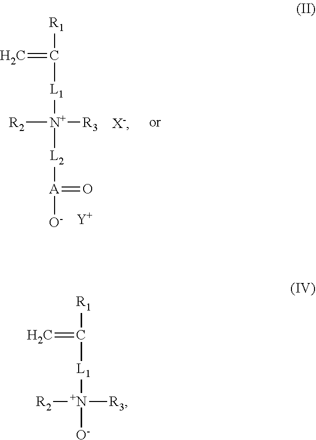

where R₁, R₂, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

According to aspects of the present disclosure, a packaged dosage unit of an implantable anti-adhesion zwitterionic composition is provided which further includes a crosslinker covalently bound to at least two of the monomeric repeating units. The crosslinker is present in an amount in the range of 0.001 wt % to 50 wt %, 0.001 wt % to 20 wt %, 0.01 wt % to 10 wt %, 0.01 wt % to 5 wt %, 0.01 wt % to 1 wt %, 0.1 wt % to 1 wt %, relative to n monomeric repeating units.

According to aspects of the present disclosure, a packaged dosage unit of an implantable anti-adhesion zwitterionic composition is provided which further includes a polymerization reaction product of a zwitterionic copolymer having reactive groups and comprising the structural formula (XI):

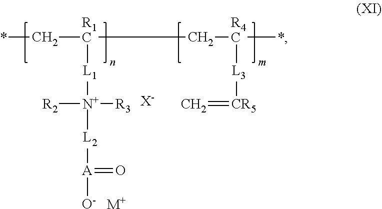

where R₁, R₂, R₃, R₄, and R₅ are each independently selected from hydrogen, alkyl, and aryl groups; L₁ is a linker that covalently couples a cationic center to the polymer backbone; L₂ is a linker that covalently couples the cationic center to an anionic group; A(═O)O⁻ is the anionic group; A is C, S, SO, P, or PO; X⁻ is the counter ion associated with the cationic center; and M⁺ is a metal ion, an ammonium ion, or an organic ion; L₃ is a linker that covalently couples a double bond to a polymer backbone, n is an integer in the range of 2 to about 100,000, m is a positive non-zero number; and m/n is in the range of 0.1%-10000%.

According to aspects of the present disclosure, a packaged dosage unit of an implantable anti-adhesion zwitterionic composition is provided according to any of the structural formulas shown herein wherein M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin.

According to aspects of the present disclosure, a packaged dosage unit of an implantable anti-adhesion zwitterionic composition is provided according to any of the structural formulas shown herein wherein A is C, S, SO, P, or PO.

According to aspects of the present disclosure, a packaged dosage unit of an implantable anti-adhesion zwitterionic composition is provided which further includes a polymerization reaction product of a zwitterionic copolymer having reactive groups and comprising the structural formula (XII):

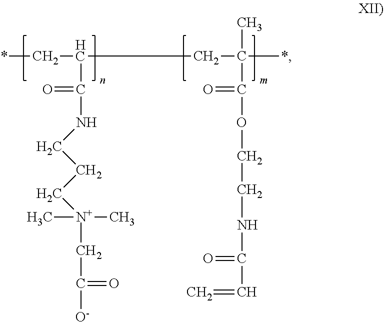

where n is an integer in the range of 2 to about 100,000, m is a positive non-zero number; and m/n is in the range of 0.1% to 10000%.

According to aspects of the present disclosure, a packaged dosage unit of an implantable anti-adhesion zwitterionic composition is provided according to any of the structural formulas shown herein wherein m/n is in the range of: 0.2% to 10%, 0.25% to 10%, 0.5% to 10%, 0.75% to 10%, 1% to 10%, 2% to 10%, 3% to 10%, 4% to 10%, 5% to 10%, 6% to 10%, 7% to 10%, 8% to 10%, 9% to 10%, 0.2% to 50%, 0.25% to 50%, 0.5% to 50%, 0.75% to 50%, 1% to 50%, 2% to 50%, 3% to 50%, 4% to 50%, 5% to 50%, 6% to 50%, 7% to 50%, 8% to 50%, 9% to 50%, 10% to 50%, 15% to 50% or 20% to 50%.

According to aspects of the present disclosure, a packaged dosage unit of an implantable anti-adhesion zwitterionic composition is provided which includes nanoparticles and/or microparticles of zwitterionic hydrogel. According to aspects of the present disclosure, the average particle diameter of the nanoparticles and/or microparticles of zwitterionic hydrogel is in the range of: 1 nanometer to 1000 microns, 0.01 micron to 1000 microns, 0.01 micron to 500 microns, 0.1 micron to 500 microns, 1 micron to 500 microns, 1 micron to 300 microns, 1 micron to 100 microns, 5 microns to 100 microns, 5 microns to 50 microns, 10 microns to 50 microns, 20 microns to 50 microns, 0.1 micron to 50 microns, 0.1 micron to 20 microns, 0.1 micron to 10 microns, or 0.1 micron to 1 micron.

According to aspects of the present disclosure a zwitterionic polymer and/or copolymer included in an implantable anti-adhesion zwitterionic composition in a packaged dosage unit has an average molecular weight in the range of: 1 kDa to 100 kDa, 10 kDa to 100 kDa, 20 kDa to 80 kDa, or 30 kDa to 70 kDa.

According to aspects of the present disclosure a zwitterionic polymer and/or copolymer included in an implantable anti-adhesion zwitterionic composition in a packaged dosage unit is present in an amount in the range of: 1 wt % to 90 wt %, 1 wt % to 60 wt %, 5 wt % to 40 wt %, 10 wt % to 40 wt %, 10 wt % to 30 wt %, or 20 wt % to 30 wt %.

According to aspects of the present disclosure, nanoparticles and/or microparticles of zwitterionic hydrogel included in an implantable anti-adhesion zwitterionic composition in a packaged dosage unit are present in an amount in the range of: 0.01 wt % to 40 wt %, 0.1 wt % to 40 wt %, 1 wt % to 30 wt %, 1 wt % to 20 wt %, 2 wt % to 10 wt %, 2 wt % to 6 wt %, relative to the aqueous liquid.

An included implantable anti-adhesion zwitterionic composition is biodegradable and biologically compatible.

An implantable anti-adhesion zwitterionic composition is provided according to aspects of the present disclosure including: an aqueous liquid and a zwitterionic polymer or copolymer, the zwitterionic polymer or copolymer comprising a plurality of repeating units, where each repeating unit has structural formula (VI), (VIII), or (X):

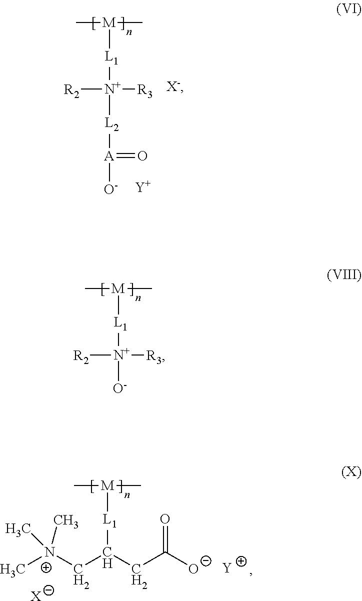

where M is a monomeric repeating unit, n is an integer from 1 to about 100,000, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═O)O if present, is an anionic group, X⁻, if present, is a counter ion associated with the cationic center, and Y+, if present, is a metal ion, an ammonium ion, or an organic ion.

The term “nanoparticles” as used herein refers to discrete particles of zwitterionic hydrogel having an average particle diameter in the nanometer (nm) range.

The term “microparticles” as used herein refers to discrete particles of zwitterionic hydrogel having an average particle diameter in the micrometer (micron, μm) range.

Nanoparticles and microparticles can have any of various shapes, such as but not limited to, spherical, oblong, or irregular.

The term “zwitterionic polymer-based cream gel” is used herein interchangeably with the term “implantable anti-adhesion zwitterionic composition which includes nanoparticles and/or microparticles of a zwitterionic hydrogel.”

An implantable anti-adhesion zwitterionic composition is provided according to aspects of the present disclosure which includes a zwitterionic polymer and/or copolymer comprises a polymerization reaction product of a zwitterionic monomer having structural formula (I), (III), or (V):

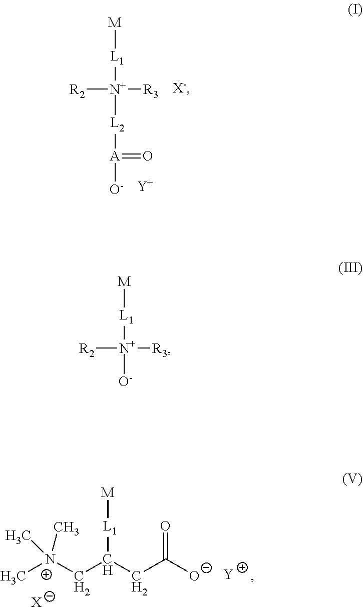

where M is a monomeric repeating unit, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

An implantable anti-adhesion zwitterionic composition is provided according to aspects of the present disclosure which includes a zwitterionic polymer and/or copolymer having a plurality of repeating units, where each repeating unit has structural formula:

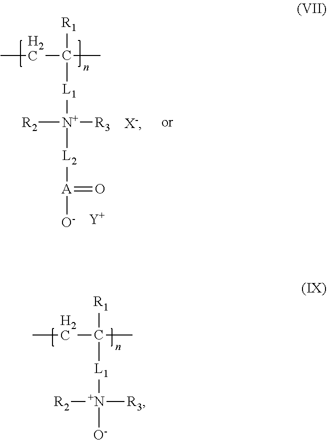

where R₁, R₂, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

An implantable anti-adhesion zwitterionic composition is provided according to aspects of the present disclosure which includes a zwitterionic polymer and/or copolymer which includes a polymerization reaction product of a zwitterionic monomer having structural formula:

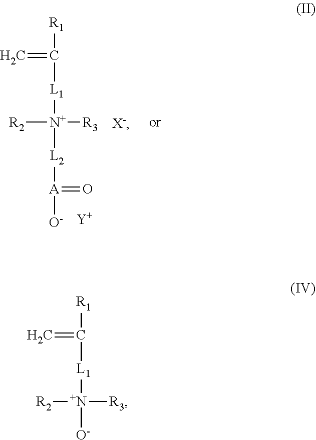

where R₁, R₂, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

An implantable anti-adhesion zwitterionic composition is provided according to aspects of the present disclosure which includes a zwitterionic polymer and/or copolymer and further includes a crosslinker covalently bound to at least two of the monomeric repeating units of the zwitterionic polymer and/or copolymer. According to aspects of the present disclosure, the crosslinker is present in an amount in the range of 0.001 wt % to 50 wt %, 0.001 wt % to 20 wt %, 0.01 wt % to 10 wt %, 0.01 wt % to 5 wt %, 0.01 wt % to 1 wt %, 0.1 wt % to 1 wt %, relative to n monomeric repeating units.

An implantable anti-adhesion zwitterionic composition is provided according to aspects of the present disclosure which further includes a polymerization reaction product of a zwitterionic copolymer having reactive groups and comprising the structural formula (XI):

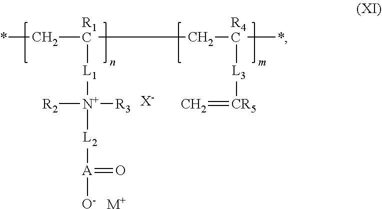

where R₁, R₂, R₃, R₄, and R₅ are each independently selected from hydrogen, alkyl, and aryl groups; L₁ is a linker that covalently couples a cationic center to the polymer backbone; L₂ is a linker that covalently couples the cationic center to an anionic group; A(═O)O⁻ is the anionic group; A is C, S, SO, P, or PO; X⁻ is the counter ion associated with the cationic center; and M⁺ is a metal ion, an ammonium ion, or an organic ion; L₃ is a linker that covalently couples a double bond to a polymer backbone, n is an integer in the range of 2 to about 100,000, m is a positive non-zero number; and m/n is in the range of 0.1%-10000%.

An implantable anti-adhesion zwitterionic composition is provided according to aspects of the present disclosure which includes a zwitterionic polymer and/or copolymer having a structural formula shown or described herein, wherein M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin.

An implantable anti-adhesion zwitterionic composition is provided according to aspects of the present disclosure which includes a zwitterionic polymer and/or copolymer having a structural formula shown or described herein, wherein A is C, S, SO, P, or PO.

An implantable anti-adhesion zwitterionic composition is provided according to aspects of the present disclosure which further includes a polymerization reaction product of a zwitterionic copolymer having reactive groups and comprising the structural formula (XII):

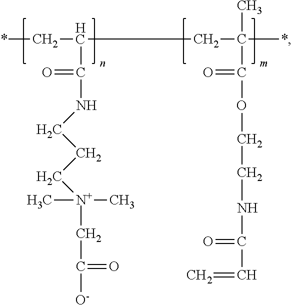

where n is an integer in the range of 2 to about 100,000, m is a positive non-zero number; and m/n is in the range of 0.1% to 10000%.

An implantable anti-adhesion zwitterionic composition is provided according to aspects of the present disclosure which further includes a polymerization reaction product of a zwitterionic copolymer having reactive groups and having a structural formula shown or described herein, wherein m/n is in the range of: 0.2% to 10%, 0.25% to 10%, 0.5% to 10%, 0.75% to 10%, 1% to 10%, 2% to 10%, 3% to 10%, 4% to 10%, 5% to 10%, 6% to 10%, 7% to 10%, 8% to 10%, 9% to 10%, 0.2% to 50%, 0.25% to 50%, 0.5% to 50%, 0.75% to 50%, 1% to 50%, 2% to 50%, 3% to 50%, 4% to 50%, 5% to 50%, 6% to 50%, 7% to 50%, 8% to 50%, 9% to 50%, 10% to 50%, 15% to 50% or 20% to 50%.

An implantable anti-adhesion zwitterionic composition is provided according to aspects of the present disclosure which includes nanoparticles and/or microparticles of a zwitterionic hydrogel, wherein the average particle diameter of the nanoparticles and/or microparticles of zwitterionic hydrogel is in the range of: 1 nanometer to 1000 microns, 0.01 micron to 1000 microns, 0.01 micron to 500 microns, 0.1 micron to 500 microns, 1 micron to 500 microns, 1 micron to 300 microns, 1 micron to 100 microns, 5 microns to 100 microns, 5 microns to 50 microns, 10 microns to 50 microns, 20 microns to 50 microns, 0.1 micron to 50 microns, 0.1 micron to 20 microns, 0.1 micron to 10 microns, or 0.1 micron to 1 micron.

An implantable anti-adhesion zwitterionic composition is provided according to aspects of the present disclosure which includes a zwitterionic polymer and/or copolymer having a structural formula shown or described herein, wherein the zwitterionic polymer and/or copolymer has an average molecular weight in the range of: 1 kDa to 100 kDa, 10 kDa to 100 kDa, 20 kDa to 80 kDa, or 30 kDa to 70 kDa.

An implantable anti-adhesion zwitterionic composition is provided according to aspects of the present disclosure which includes a zwitterionic polymer and/or copolymer having a structural formula shown or described herein, wherein the zwitterionic polymer and/or copolymer is present in the composition in an amount in the range of: 1 wt % to 90 wt %, 1 wt % to 60 wt %, 5 wt %, to 40 wt %, 10 wt % to 40 wt %, 10 wt % to 30 wt %, or 20 wt % to 30 wt %.

An implantable anti-adhesion zwitterionic composition is provided according to aspects of the present disclosure which includes nanoparticles and/or microparticles of a zwitterionic hydrogel, wherein the zwitterionic composition includes a zwitterionic polymer and/or copolymer having a structural formula shown or described herein, wherein the nanoparticles and/or microparticles of zwitterionic hydrogel are present in the composition in an amount in the range of: 0.01 wt % to 40 wt %, 0.1 wt % to 40 wt %, 1 wt % to 30 wt %, 1 wt % to 20 wt %, 2 wt % to 10 wt %, 2 wt % to 6 wt %, relative to an aqueous liquid present in the composition.

An implantable anti-adhesion zwitterionic composition is provided according to aspects of the present disclosure, wherein the zwitterionic composition includes a zwitterionic polymer and/or copolymer having a structural formula shown or described herein, wherein the implantable anti-adhesion zwitterionic composition is biodegradable and pharmaceutically acceptable.

Methods of inhibiting tissue adhesion in a subject in need thereof are provided according to aspects of the present disclosure which include: administering an effective amount of an implantable anti-adhesion zwitterionic composition to a tissue surface of the subject in need thereof. According to particular aspects, the tissue surface is exposed by surgery. Such surgeries are not limited to any particular type of surgery and can be any type of surgery in which formation of undesired tissue adhesions is a concern.

Administration to a tissue surface can be by extrusion of the implantable anti-adhesion zwitterionic composition from a packaged dosage unit onto the tissue surface. Alternatively, the implantable anti-adhesion zwitterionic composition can be administered from a bulk supply.

Methods of inhibiting tissue adhesion in a subject in need thereof are provided according to aspects of the present disclosure which include: administering an effective amount of an implantable anti-adhesion zwitterionic composition to a tissue surface of the subject in need thereof, wherein the implantable anti-adhesion zwitterionic composition requires no curing following administration.

Methods of inhibiting tissue adhesion in a subject in need thereof are provided according to aspects of the present disclosure which include: administering an effective amount of an implantable anti-adhesion zwitterionic composition to a tissue surface of the subject in need thereof, wherein the implantable anti-adhesion zwitterionic composition undergoes no polymerization or crosslinking reaction following administration.

Methods of inhibiting tissue adhesion in a subject in need thereof are provided according to aspects of the present disclosure which include: administering an effective amount of an implantable anti-adhesion zwitterionic composition to a tissue surface of the subject in need thereof, wherein the implantable anti-adhesion zwitterionic composition is flowable and/or malleable, and conforms to the tissue surface.

Methods of inhibiting tissue adhesion in a subject in need thereof are provided according to aspects of the present disclosure which include: administering an effective amount of an implantable anti-adhesion zwitterionic composition to a tissue surface of the subject in need thereof, wherein the implantable anti-adhesion zwitterionic composition does not undergo further chemical reaction which leads to an increase of the MW of the composition.

According to aspects of the present disclosure, the zwitterionic polymer is a polymerization product of zwitterionic monomers. According to aspects of the present disclosure, the zwitterionic polymer further contains amide moieties in the polymer sidechain and/or the polymer backbone. According to aspects of the present disclosure, the zwitterionic polymer is a copolymer which is a reaction product of zwitterionic monomers and amide-containing monomers.

Zwitterionic Monomers

According to aspects described herein, the zwitterionic monomer has the structural formula (I):

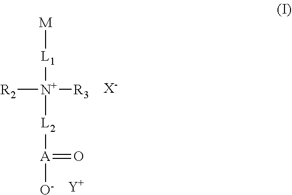

where M is a monomeric repeating unit, According to aspects of the present invention, M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin. According to aspects of the present invention, R₂, and R₃ are each independently selected from hydrogen, alkyl, and aryl groups; L₁ is a linker that covalently couples a cationic center to a polymer backbone; L₂ is a linker that covalently couples the cationic center to an anionic group; A(═O)O⁻ is the anionic group; X⁻ is a counter ion associated with the cationic center; and Y+ is a metal cation, an ammonium cation, or an organic cation.

According to aspects described herein, the zwitterionic monomer has the structural formula (II):

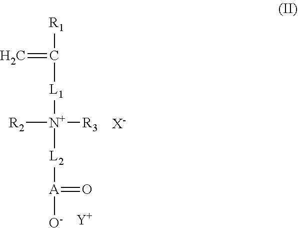

where R₁, R₂, and R₃ are each independently selected from hydrogen, alkyl, and aryl groups; L₁ is a linker that covalently couples a cationic center to a polymer backbone; L₂ is a linker that covalently couples the cationic center to an anionic group; A(═O)O⁻ is the anionic group; X⁻ is a counter ion associated with the cationic center; and Y+ is a metal cation, an ammonium cation, or an organic cation.

According to aspects of the present disclosure, the zwitterionic monomer has the structural formula (III):

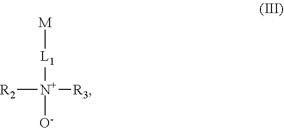

where M is a monomeric repeating unit. According to aspects of the present invention, M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin. According to aspects of the present invention, R₂ and R₃ in structural formulas shown herein representative alkyl groups include C₁-C₃₀ straight chain and branched alkyl groups. According to aspects of the present disclosure, the alkyl group is further substituted with one of more substituents including, for example, an aryl group (e.g., —CH₂C₆H₅, benzyl). L₁ is a linker that covalently couples a cationic center to a polymer backbone.

According to aspects of the present disclosure, the zwitterionic monomer has the structural formula (IV):

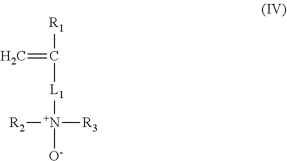

For each R₁, R₂, and R₃ in structural formulas shown herein representative alkyl groups include C₁-C₃₀ straight chain and branched alkyl groups. According to aspects of the present disclosure, the alkyl group is further substituted with one of more substituents including, for example, an aryl group (e.g., —CH₂C₆H₅, benzyl).

For each R₁, R₂, and R₃ in structural formulas shown herein representative aryl groups include C₆-C₁₂ aryl groups including, for example, phenyl including substituted phenyl groups (e.g., benzoic acid).

For each R₁, R₂, and R₃ in structural formulas shown herein representative alkyl groups include O—C₁₀ straight chain and branched alkyl groups.

According to aspects of the present disclosure, the alkyl group is further substituted with one of more substituents including, for example, an aryl group (e.g., —CH₂CH₅H₆, benzyl).

According to aspects of the present disclosure, R₂ and R₃ in structural formulas shown herein are methyl.

According to aspects of the present disclosure, R₁, R₂, and R₃ in structural formulas shown herein are methyl.

According to aspects of the present disclosure, R₂ and R₃ are taken together with N⁺ form the cationic center in structural formulas shown herein.

According to aspects of the present disclosure, L₁ includes a functional group (e.g., ester or amide) that couples the remainder of L₁ to the C═C double bond for the monomers, or the backbone for the polymers. In addition to the functional group, L₁ can include a C₁-C₂₀ alkylene chain. Representative L₁ groups include —C(═O)O—(CH₂)ₙ— and —C(═O)NH—(CH₂)n-, where n is 1-20 (e.g., n:=2).

L₂ can be a C₁-C₂₀ alkylene chain according to aspects of the present disclosure. Representative L₂ groups include —(CH₂)ₙ—, where n is 1-20 (e.g., 1, 2, or 3).

A(═O)O⁻ is an anionic group in structural formulas shown herein. The group is a carboxylic acid (where A is C), a sulfmic acid (where A is S), a sulfonic acid (where A is SO), a phosphinic acid (where A is P), or a phosphonic acid (where A is PO).

As noted, X⁻ in structural formula shown herein is the counter ion associated with the cationic center. The counter ion can be the counter ion that results from the synthesis of the cationic polymers or the monomers (e.g., Cl⁻, Br⁻, I⁻). The counter ion that is initially produced from the synthesis of the cationic center can also be exchanged with other suitable counter ions to provide polymers having controllable hydrolysis properties and other biological properties. According to aspects of the present disclosure, representative hydrophobic counterions include carboxylates, such as benzoic acid and fatty acid anions (e.g., CH₃(CH₂)ₙCO₂⁻ where n can be from 1 to 19); alkyl sulfonates (e.g., CH₃(CH₂)ₙSO₃⁻ where n can be from 1 to 19); salicylate; lactate; bis(trifluoromethylsulfonyl)amide anion (N⁻(SO₂CF₃)₂); and derivatives thereof. Other counter ions also can be chosen from chloride, bromide, iodide, sulfate; nitrate; perchlorate (CIO₄); tetrafluoroborate (BF₄) hexafluorophosphate (PF₆); trifluoromethylsulfonate (SO₃CF₃); and derivatives thereof. Other suitable counter ions include salicylic acid (2-hydroxybenzoic acid), benzoate, and lactate.

Y+ in structural formulas (I) and (II) is a metal ion, an ammonium ion, or an organic ion.

In structural formulas shown herein N⁺ is the cationic center. In certain embodiments, the cationic center is a quaternary ammonium (N bonded to L₁, R₂, R₃, and L₂). In addition to ammonium, other useful cationic centers (R₂ and R₃ taken together with N) include imidazolium, triazaolium, pyridinium, morpholinium, oxazolidinium, pyrazinium, pyridazinium, pyrimidinium, piperazinium, and pyrrolidinium.

According to aspects of the present disclosure, R₁, R₂, and R₃ in structural formulas shown herein are independently selected from the group consisting of C₁-C₃ alkyl. In one embodiment, R₁, R₂, and R₃ in structural formulas shown herein are methyl.

According to aspects of the present disclosure, L₁ in structural formulas shown herein is selected from the group consisting of —C(═O)O—(CH₂)n- and —C(═O)NH—(CH₂)n-, wherein n is 1-20. In one embodiment, L₁ in structural formulas shown herein is —C(═O)O—(CH₂)₂—. In another embodiment, L₁ in structural formulas shown herein is —C(═O)NH—(CH₂)₃—.

According to aspects of the present disclosure, L₂ in structural formulas shown herein is —(CH₂)n-, where n is an integer from 1-20. In one embodiment, L₂ in structural formulas shown herein is —(CH₂)—. In another embodiment, L₂ in structural formulas shown herein is —(CH₂)₂—.

According to aspects of the present disclosure, R₁, R₂, and R₃ in structural formulas shown herein are methyl, L₁ in structural formulas shown herein is —C(═O)NH—(CH₂)₃—, L₂ in structural formulas shown herein is —(CH₂)₂—, A is C.

According to aspects of the present disclosure, the zwitterionic monomer has the structural formula (V):

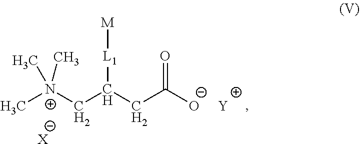

where M is a monomeric repeating unit, L₁ is a linker, X− is a counter ion associated with a cationic center of structure (V) and Y+ is a counter ion associated with an anionic center of structure (V). According to aspects of the present invention, M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin. According to aspects of the present invention, L1 is —C(═O)O—(CH₂)ₙ₁— or —C(═O)NH—(CH₂)ₙ₁—, where n1 is an integer from 0 to 20, such as where n1 is 0, 1, 2, 3, 4, 5, 6, 7, 8, 9, 10, 11, 12, 13, 14, 15, 16, 17, 18, 19, or 20.

Zwitterionic monomers can be obtained by synthesis and/or are commercially available.

Zwitterionic monomers containing carboxybetaine and sulfobetaine can be synthesized by using a tertiary amine containing acrylate, acrylamide, or vinyl monomer to react with lactone or sultone, or to react with alkyl halides containing acid groups, or to react with alkyl halides containing acid esters followed by removal acid ester to give acid groups.

Regarding zwitterionic monomers and zwitterionic polymers, U.S. Patent Application Publication 2012/0322939 is hereby incorporated by reference in its entirety and particularly sections 0081-0159 describing zwitterionic monomers and zwitterionic polymers included in compositions and methods according to aspects of the present disclosure.

Zwitterionic Polymers

An included zwitterionic polymer according to aspects of the present disclosure has a plurality of repeating units, where each repeating unit has structural formula (VI):

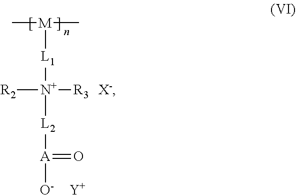

where M is a monomeric repeating unit, n is an integer from 1 to about 100,000. According to aspects of the present invention, M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin.

An included zwitterionic polymer according to aspects of the present disclosure has a plurality of repeating units, where each repeating unit has structural formula (VII):

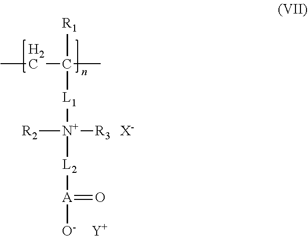

An included zwitterionic polymer according to aspects of the present disclosure has a plurality of repeating units, where each repeating unit has structural formula (VIII):

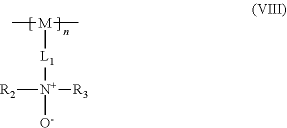

An included zwitterionic polymer according to aspects of the present disclosure has a plurality of repeating units, where each repeating unit has structural formula (IX):

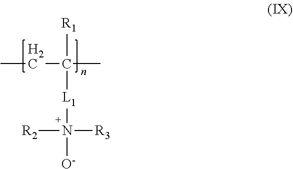

For each R₁, R₂, and R₃ in structural formulas of zwitterionic polymers shown herein, representative alkyl groups include C₁-C₃₀ straight chain and branched alkyl groups. According to aspects of the present disclosure, the alkyl group is further substituted with one of more substituents including, for example, an aryl group (e.g., —CH₂C₆H₅, benzyl).

For each R₁, R₂, and R₃ in structural formulas of zwitterionic polymers shown herein representative aryl groups include C₆-C₁₂ aryl groups including, for example, phenyl including substituted phenyl groups (e.g., benzoic acid).

For each R₁, R₂, and R₃ in structural formulas of zwitterionic polymers shown herein representative alkyl groups include C₁-C₁₀ straight chain and branched alkyl groups. According to aspects of the present disclosure, the alkyl group is further substituted with one of more substituents including, for example, an aryl group (e.g., —CH₂C₆H₅, benzyl).

According to aspects of the present disclosure, R₂ and R₃ in structural formula (VI) and (VIII) shown herein are methyl.

According to aspects of the present disclosure, R₁, R₂, and R₃ in structural formula (VII) and (IX) shown herein are methyl.

According to aspects of the present disclosure, R₂ and R₃ are taken together with N+ form the cationic center in structural formula (VI) and (VII) shown herein.

Y+ in structural formula (VI) and (VII) is a metal ion, an ammonium ion, or an organic ion.

An included zwitterionic polymer according to aspects of the present disclosure is a carnitine-derived zwitterionic polymer having a plurality of repeating units, where each repeating unit has structural formula (X):

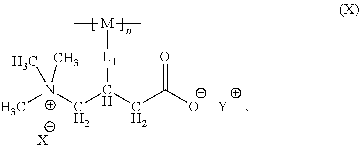

where M is a monomeric repeating unit, L₁ is a linker, n is an integer from 1 to about 10000, X⁻ is a counter ion associated with the cationic center, and Y⁺ is a counter ion associated with the anionic center. According to aspects of the present invention, M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin.

According to aspects of the present disclosure, L₁ includes a functional group (e.g., ester or amide) that couples the remainder of L₁ to the C═C double bond for the monomers, or the backbone for the polymers. In addition to the functional group, L₁ can include a C₁-C₂₀ alkylene chain. Representative L₁ groups include —C(═O)O—(CH₂)ₙ— and —C(═O)NH—(CH₂)ₙ—, where n is 0-20 (e.g., n=3). According to aspects of the present invention, L₁ is —C(═O)O—(CH₂)ₙ₁— or —C(═O)NH—(CH₂)ₙ₁—, where n1 is an integer from 0 to 20, such as where n1 is 0, 1, 2, 3, 4, 5, 6, 7, 8, 9, 10, 11, 12, 13, 14, 15, 16, 17, 18, 19, or 20.

L₂ can be a C₁-C₂₀ alkylene chain according to aspects of the present disclosure. Representative L₂ groups include —(CH₂)ₙ—, where n is 1-20 (e.g., 1, 2, or 3).

A(═O)O− is an anionic group in structural formulas shown herein. The group is a carboxylic acid (where A is C), a sulfinic acid (where A is S), a sulfonic acid (where A is SO), a phosphinic acid (where A is P), or a phosphonic acid (where A is PO).

As noted, X⁻ in structural formulas shown herein is the counter ion associated with the cationic center. The counter ion can be the counter ion that results from the synthesis of the cationic polymers or the monomers (e.g., Cl⁻, Br⁻, I⁻). The counter ion that is initially produced from the synthesis of the cationic center can also be exchanged with other suitable counter ions to provide polymers having controllable hydrolysis properties and other biological properties. According to aspects of the present disclosure, representative hydrophobic counter ions include carboxylates, such as benzoic acid and fatty acid anions (e.g., CH₃(CH₂)ₙCO₂⁻ where n=1-19); alkyl sulfonates (e.g., CH₃(CH₂)ₙSO₃⁻ where n=1-19); salicylate; lactate; bis(trifluoromethylsulfonyl)amide anion (N⁻(SO₂CF₃)₂); and derivatives thereof. Other counter ions also can be chosen from chloride, bromide, iodide, sulfate; nitrate; perchlorate (ClO₄); tetrafluoroborate (BF₄); hexafluorophosphate (PF₆); trifluoromethylsulfonate (SO₃CF₃); and derivatives thereof. Other suitable counter ions include salicylic acid (2-hydroxybenzoic acid), benzoate, and lactate.

According to aspects, R₁, R₂, and R₃ are each independently selected from hydrogen, alkyl, and aryl groups; L₁ is a linker that covalently couples a cationic center to a polymer backbone; L₂ is a linker that covalently couples the cationic center to an anionic group; A(═O)O⁻ is the anionic group; A is C, S, SO, P, or PO; X⁻ is a counter ion associated with the cationic center; Y⁺ is a counter ion associated with the (A=O)O— anionic center; and n is an integer in the range of 2 to about 100,000.

An included zwitterionic polymer according to aspects of the present disclosure has a plurality of repeating units, where each repeating unit has structural formula (VII), where R₁ is selected from the group consisting of hydrogen, fluorine, trifluoromethyl, C₁-C₆ alkyl, and C₆-C₁₂ aryl groups; R₂ and R₃ are independently selected from the group consisting of alkyl and aryl, or taken together with a nitrogen to which they are attached form a cationic center; L₁ is a linker that covalently couples the cationic center [N⁺(R₂)(R₃)] to a monomer double bond or its polymer backbone [—(CH₂—CR₁)n-]; L₂ is a linker that covalently couples an anionic center [A(═O)—O—] to the cationic center; A is C, S, SO, P, or PO; Y⁺ is a metal ion, an ammonium ion, or an organic ion; X⁻ is a counter ion associated with the cationic center; and n is an integer in the range of 2 to about 100,000.

An included zwitterionic polymer according to aspects of the present disclosure according to aspects of the present disclosure has a plurality of repeating units selected from the group consisting of: a sulfobetaine acrylate, a sulfobetaine methacrylate, a sulfobetaine acrylamide, a sulfobetaine methacrylamide, a sulfobetaine vinyl compound, a carboxybetaine acrylate, a carboxybetaine methacrylate, a carboxybetaine acrylamide, a carboxybetaine methacrylamide, a carboxybetaine vinyl compound, a phosphobetaine acrylate, a phosphobetaine methacrylate, a phosphobetaine acrylamide, a phosphobetaine methacrylamide, a phosphobetaine vinyl compound; and a mixture of any two or more thereof.

An included zwitterionic polymer according to aspects of the present disclosure is selected from the group consisting of: a sulfobetaine acrylate polymer, a sulfobetaine methacrylate polymer, a sulfobetaine acrylamide polymer, a sulfobetaine methacrylamide polymer, a sulfobetaine vinyl polymer, a carboxybetaine acrylate polymer, a carboxybetaine methacrylate polymer, a carboxybetaine acrylamide polymer, a carboxybetaine methacrylamide polymer, a carboxybetaine vinyl polymer, a phosphobetaine acrylate polymer, a phosphobetaine methacrylate polymer, a phosphobetaine acrylamide polymer, a phosphobetaine methacrylamide polymer, a phosphobetaine vinyl polymer; a polymer comprising of two or more zwitterionic repeating units selected from the group consisting of: a sulfobetaine acrylate, a sulfobetaine methacrylate, a sulfobetaine acrylamide, a sulfobetaine methacrylamide, a sulfobetaine vinyl compound, a carboxybetaine acrylate, a carboxybetaine methacrylate, a carboxybetaine acrylamide, a carboxybetaine methacrylamide, a carboxybetaine vinyl compound, a phosphobetaine acrylate, a phosphobetaine methacrylate, a phosphobetaine acrylamide, a phosphobetaine methacrylamide, a phosphobetaine vinyl compound; and a mixture of any two or more zwitterionic polymers thereof.

An included zwitterionic polymer according to aspects of the present disclosure is selected from the group consisting of: PCBAA, PMCBAA, PCBAA-1; PCBMA, PSBMA, PMPC, PCBOH, PCAR, and a mixture of any two or more thereof.

In structural formulas herein N⁺ is the cationic center. In certain embodiments, the cationic center is a quaternary ammonium (N bonded to L₁, R₂, R₃, and L₂). In addition to ammonium, other useful cationic centers (R₂ and R₃ taken together with N) include imidazolium, triazaolium, pyridinium, morpholinium, oxazolidinium, pyrazinium, pyridazinium, pyrimidinium, piperazinium, and pyrrolidinium.

A zwitterionic polymer formed from a zwitterionic monomer in structural formulas shown herein can have 2 to about 100,000 monomer units per polymer chain.

According to aspects of the present disclosure, the molecular weight of the zwitterionic polymer is in the range of 5 kDa to 200 kDa. According to aspects of the present disclosure, the molecular weight of the zwitterionic polymer in the range of 5 kDa to 100 kDa. According to aspects of the present disclosure, the molecular weight of the zwitterionic polymer is in the range of 5 kDa to 70 kDa. According to aspects of the present disclosure, the molecular weight of the zwitterionic polymer is in the range of 5 kDa to 50 kDa. According to aspects of the present disclosure, the molecular weight of the zwitterionic polymer is in the range of 1 kDa to 100 kDa, 10 kDa to 100 kDa, 20 kDa to 80 kDa, or 30 kDa to 70 kDa. According to aspects of the present disclosure, zwitterionic polymer described herein is preferred to have a non-zero molecular weight not far above, near to, or below the molecular weight cut-off for glomerular filtration (˜70 kDa) to facilitate clearance from the body of a subject to whom the zwitterionic polymer was administered. According to aspects of the present disclosure, a zwitterionic polymer described herein can be degradable under physiological conditions to facilitate the clearance from the body of a subject to whom the zwitterionic polymer was administered. Degradability of the polymer can be facilitated by various structural features such as, but not limited to, a hydrolysable moiety and/or a reducible moiety, upon the cleavage of which the molecular weight of the polymer is reduced. The zwitterionic polymer can have hydrolysable moieties including but not limited to a carboxylate ester, a phosphate ester, a carbamate, an anhydride, an acetal, a ketal, an acyloxyalkyl ether, an imine, a hydrazone, an orthoester, a thioester, a carbonate, a sulfonate, a peptide, an oligonucleotide, etc. The zwitterionic polymer can have reducible moieties including but not limited to a disulfide, etc.

According to aspects of the present disclosure, R₁, R₂, and R₃ in structural formulas shown herein are independently selected from the group consisting of C₁-C₃ alkyl. In one embodiment, R₁, R₂, and R₃ in structural formulas shown herein are methyl.

According to aspects of the present disclosure, L₁ in structural formulas shown herein is selected from the group consisting of —C(═O)O—(CH₂)n- and —C(═O)NH—(CH₂)n-, wherein n is 0-20. In one embodiment, L₁ in structural formulas shown herein is —C(═O)NH—(CH₂)ₙ—.

According to aspects of the present disclosure, L₂ in structural formulas shown herein is —(CH₂)n-, where n is an integer from 1-20. In one embodiment, L₂ in structural formula (VII) shown herein is —(CH₂)₂—.

According to aspects of the present disclosure, R₁, R₂, and R₃ in structural formulas shown herein are methyl, L₁ in structural formulas shown herein is —C(═O)NH—(CH₂)₃—, L₂ in structural formulas shown herein is —(CH₂)₂—, A is C.

According to aspects of the present disclosure, R₁ in structural formulas shown here is hydrogen, R₂, and R₃ in structural formulas shown herein are methyl, L₁ in structural formulas shown herein is —C(═O)NH—(CH₂)₃—, L₂ in structural formulas shown herein is —(CH₂)₂—, A is C.

According to aspects, the zwitterionic polymer is selected from the group consisting of: poly(3-((3-acrylamidopropyl)dimethylammonio)propanoate) (PCBAA), poly(2-((3-acrylamidopropyl)dimethylammonio)acetate) (PCBAA-1). poly(3-((3-methacrylamidopropyl)dimethylammonio)propanoate) (PMCBAA), poly(3-[[2-(methacryloyloxy)ethyl]dimethylammonio]propionate) (PCBMA); poly(N-(carboxymethyl)-2-hydroxy-N,N-dimethyl-3-[(2-methyl-1-oxo-2-propen-1-yl)oxy]-1-propanaminium) (PCBOH); poly[2-(methacryloyloxy)ethyl]dimethyl-(3-sulfopropyl)ammonium hydroxide (PSBMA); poly(((2R)-3-carboxy-N,N,N-trimethyl-2-[(1-oxo-2-propen-1-yl)oxy]-1-propanaminium) (PCAR); and poly(methacryloyloxyethyl phosphorylcholine) (PMPC).

According to aspects, the zwitterionic polymer is PCBAA.

According to aspects, the zwitterionic polymer is PMCBAA

According to aspects, the zwitterionic polymer is PCBAA-1.

Copolymers

An included copolymer according to aspects of the present disclosure is a polymerization product of a zwitterionic monomer and an amide containing monomer.

An amide-containing monomer can be selected from, but not limited to, acrylamide, methacrylamide, N,N-dimethylacrylamide, N-3-(Dimethylamino)propylmethacrylamide, N-Ethylacrylamide, N-tert-Butylacrylamide, N-Hydroxyethyl acrylamide, N-(Hydroxymethyl)acrylamide, N-acryloyl glycinamide, N-(2-hydroxypropyl) methacrylamide, N-Isopropylacrylamide, N-Tris(hydroxymethyl)methylacrylamide, 2-Acrylamido-2-methyl-1-propanesulfonic acid, [3-(Methacryloylamino)propyl]trimethylammonium chloride, one or more of above.

A copolymer formed from one or more zwitterionic monomers and one or more amide-containing monomers according to aspects of the present disclosure can have 2 to about 100,000 monomer unit per polymer chain.

According to aspects of the present disclosure, the molecular weight of the copolymer is in the range of 5 kDa to 200 kDa. According to aspects of the present disclosure, the molecular weight of the copolymer in the range of 5 kDa to 100 kDa. According to aspects of the present disclosure, the molecular weight of the copolymer is in the range of 5 kDa to 70 kDa. According to aspects of the present disclosure, the molecular weight of the copolymer is in the range of 5 kDa to 50 kDa. According to aspects of the present disclosure, the molecular weight of the zwitterionic copolymer is in the range of 1 kDa to 100 kDa, 10 kDa to 100 kDa, 20 kDa to 80 kDa, or 30 kDa to 70 kDa. According to aspects of the present disclosure, zwitterionic copolymer described herein is preferred to have a non-zero molecular weight not far above, near to or below the molecular weight cut-off for glomerular filtration (˜70 kDa) to facilitate clearance from the body of a subject to whom the zwitterionic copolymer was administered. According to aspects of the present disclosure, zwitterionic copolymer described herein can be degradable under physiological conditions to facilitate the clearance from the body of a subject to whom the zwitterionic copolymer was administered. Degradability of the copolymer can be facilitated by various structural features such as, but not limited to, a hydrolysable moiety and/or a reducible moiety, upon the cleavage of which the molecular weight of the copolymer is reduced. The zwitterionic copolymer can have hydrolysable moieties including but not limited to a carboxylate ester, a phosphate ester, a carbamate, an anhydride, an acetal, a ketal, an acyloxyalkyl ether, an imine, a hydrazone, an orthoester, a thioester, a carbonate, a sulfonate, a peptide, an oligonucleotide, etc. The zwitterionic copolymer can have reducible moieties including but not limited to a disulfide, etc.

Polymerization Reactions

According to aspects of the present disclosure, polymerization of monomers and crosslinkers according to aspects shown or described herein will result in polymer backbones including vinyl backbones (i.e., —C(R′)(R″)—C(R′″)(R″″)—, where R′, R″, R′″, and R′″ are independently selected from hydrogen, alkyl, and aryl) derived from vinyl monomers (e.g., acrylate, methacrylate, acrylamide, methacrylamide, styrene).

Methods of polymerizing reaction components having reactive groups to produce a polymerization product include radical polymerization, living polymerization, condensation, ring opening polymerization and click chemistry. Details of polymerization mechanisms are well-known along with appropriate reaction conditions, initiators, catalysts and other standard co-factors as exemplified herein.

Zwitterionic polymers (linear zwitterionic polymers) and copolymers thereof, are synthesized through free radical polymerization method or living polymerization method. These polymerization methods normally involve initiators, zwitterionic monomers, amide-containing monomers (optional), catalysts (optional), and the polymerization condition is selected from heating, lighting, etc. The feeding monomer amount relative to initiator amount is varied to obtain polymers with different molecular weight (MW). The obtained polymers are typically purified by dialyzing against water followed by freeze-drying.

Zwitterionic Hydrogel Nanoparticles and Zwitterionic Hydrogel Microparticles

According to aspects of the present disclosure, zwitterionic hydrogel nanoparticles and zwitterionic hydrogel microparticles are chemical hydrogel particles selected from the group consisting of: a polymerization product of a zwitterionic monomer included in this disclosure and a non-zwitterionic crosslinker; a polymerization product of a zwitterionic monomer included in this disclosure and a zwitterionic copolymer comprising one or more reactive groups reactive with the zwitterionic monomer; and a polymerization product of a first zwitterionic copolymer comprising reactive functional groups and a second zwitterionic copolymer comprising reactive functional groups, wherein the first and second zwitterionic copolymers are identical or different.

A zwitterionic monomer included in zwitterionic hydrogel nanoparticles and zwitterionic hydrogel microparticles according to aspects of the present disclosure is described herein. A zwitterionic monomer included in zwitterionic hydrogel nanoparticles and zwitterionic hydrogel microparticles according to aspects of the present disclosure is selected from the group consisting of: a sulfobetaine acrylate, a sulfobetaine methacrylate, a sulfobetaine acrylamide, a sulfobetaine methacrylamide, a sulfobetaine vinyl compound, a carboxybetaine acrylate, a carboxybetaine methacrylate, a carboxybetaine acrylamide, a carboxybetaine methacrylamide, a carboxybetaine vinyl compound, a phosphobetaine acrylate, a phosphobetaine methacrylate, a phosphobetaine acrylamide, a phosphobetaine methacrylamide, a phosphobetaine vinyl compound; and a mixture of any two or more thereof.

A non-zwitterionic crosslinker reacted with a zwitterionic monomer according to aspects of the present disclosure is a polyreactive crosslinking agent. According to particular aspects, a non-zwitterionic crosslinker is an acryloyl-containing crosslinker. According to particular aspects, a non-zwitterionic crosslinker is an allyl crosslinker. According to particular aspects, a non-zwitterionic crosslinker is a vinyl compound.

A non-zwitterionic crosslinker reacted with a zwitterionic monomer according to aspects of the present disclosure is one or more of: allyl methacrylate, diallyl itaconate, monoallyl itaconate, dially maleate, diallyl fumarate, diallyl succinate, diallyl phthalate, triallyl cyanurate, triallyl isocyanurate, diethylene glycol bis-allyl carbonate, divinyl ether of diethylene glycol, triallyl phosphate, triallyl trimellitate, allyl ether, diallylimidazolidone, pentaerythritol triallyl ether (PETE), N,N-diallylmelamine, triallyl-1,3,5-triazine-2,4,6-(1H,3H,5H)trione (TATT), 2,4,6-Triallyloxy-1,3,5-triazine; vinyl compounds, e.g. divinyl benzene, N,N′-methylene bis acrylamide (MBAA), methylenebis(methacrylamide), ethylene glycol dimethacrylate, ethylene glycol diacrylate, neopentylglycol dimethacrylate, tetraethylene glycol dimethacrylate, polyethylene glycol dimethacrylate, polyethylene glycol diacrylate, hexamethylene bis maleimide, divinyl urea, bisphenol A bis methacrylate, divinyl adipate, glycerin trimethacrylate, trimethylolpropane triacrylate, trivinyl trimellitate, 1,5-pentadiene, 1,7-octadiene, 1,9-decadiene, 1,3-bis(4-methacryloxybutyl) tetramethyl disiloxane, divinyl ether, divinyl sulfone, N-vinyl-3(E)-ethylidene pyrrolidone (EVP), ethylidene bis(N-vinyl pyrrolidone) (EBVP).

A non-zwitterionic crosslinker reacted with a zwitterionic monomer according to aspects of the present disclosure is MBAA.

According to aspects of the present disclosure, zwitterionic hydrogel nanoparticles and zwitterionic hydrogel microparticles include a polymerization product of a zwitterionic monomer described herein and a non-zwitterionic crosslinker which is degradable under physiological conditions. Upon the degradation of the non-zwitterionic crosslinker, the formed hydrogel particles disintegrate, facilitating further degradation and/or clearance from the body of a subject to whom the zwitterionic nanohydrogel and microhydrogel particles were administered. Degradability of the non-zwitterionic crosslinker may be facilitated by various structural features such as, but not limited to, a hydrolysable moiety and/or a reducible moiety. The non-zwitterionic crosslinker may have hydrolysable moieties including but not limited to a carboxylate ester, a phosphate ester, a carbamate, an anhydride, an acetal, a ketal, an acyloxyalkyl ether, an imine, a hydrazone, an orthoester, a thioester, a carbonate, a sulfonate, a peptide, an oligonucleotide, etc. The non-zwitterionic crosslinker may have reducible moieties including but not limited to a disulfide, etc.

In preferred aspects, the non-zwitterionic crosslinker is an acrylate, methacrylate, acrylamide, or methacrylamide degradable non-zwitterionic crosslinker.

In preferred aspects, the non-zwitterionic crosslinker is N,N′-bis(acryloyl)cystamine.

A zwitterionic copolymer having reactive groups according to aspects of the present disclosure has the structural formula (XI):

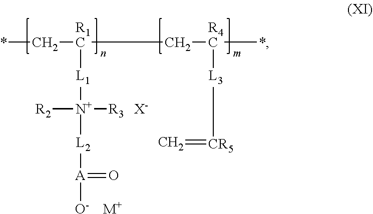

where R₁, R₂, R₃, R₄, and R₅ are each independently selected from hydrogen, alkyl, and aryl groups; L₁ is a linker that covalently couples a cationic center to the polymer backbone; L₂ is a linker that covalently couples the cationic center to an anionic group; A(═O)O⁻ is the anionic group; A is C, S, SO, P, or PO; X⁻ is the counter ion associated with the cationic center; and M⁺ is a metal ion, an ammonium ion, or an organic ion; L₃ is a linker that covalently couples a double bond to a polymer backbone, n is an integer in the range of 2 to about 100,000, m is a positive non-zero number; and m/n is in the range of 0.1%-10000%.

A zwitterionic copolymer having reactive groups according to aspects of the present disclosure has the structural formula: (XI), where R₁, R₂, R₃, R₄, and R₅ are each independently selected from hydrogen, alkyl, and aryl groups; R₁, R₄, and R₅ are each independently selected from the group consisting of hydrogen, fluorine, trifluoromethyl, C₁-C₆ alkyl, and C₆-C₁₂ aryl groups; R₂ and R₃ are independently selected from the group consisting of alkyl and aryl, or taken together with the nitrogen to which they are attached form a cationic center; L₁ is a linker that covalently couples a cationic center [N*(R₂)(R₃)] to a monomer double bond or its polymer backbone [—(CH₂—CR₁)ₙ—]; L₂ is a linker that covalently couples a anionic center [A(═O)—O⁻] to a cationic center; A is C, S, SO, P, or PO; M⁺ is a counter ion associated with the (A=O)O⁻ anionic center; X⁻ is a counter ion associated with the cationic center; L₃ is a linker that covalently couples a double bond to a polymer backbone; n is an integer in the range of 2 to about 100,000, m is a positive non-zero number; and m/n is in the range of 0.1%-10000%.

A zwitterionic copolymer having reactive groups according to aspects of the present disclosure is a PCBAA-1 macrocrosslinker having the structural formula:

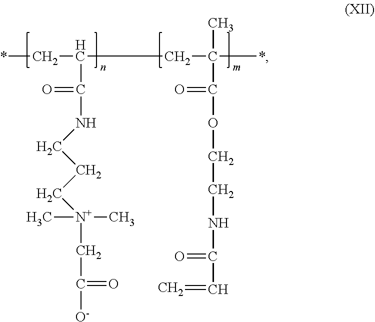

where n is an integer in the range of 2 to about 100,000, m is a positive non-zero number; and m/n is in the range of 0.1%-10000%. According to aspects of the present disclosure, m/n is in the range of 0.2%-50%, 0.25%-50%, 0.5%-50%, 0.75%-50%, 1%-50%, 2%-50%, 3%-50%, 4%-50%, 5%-50%, 6%-50%, 7%-50%, 8%-50%, 9%-50%, 10%-50%, 15%-50% or 20%-50%.

According to aspects, the zwitterionic hydrogel nanoparticles and zwitterionic hydrogel microparticles are synthesized through free radical polymerization method or living polymerization method. These polymerization methods normally involve initiators, zwitterionic monomers, crosslinkers, surfactants (optional; to form emulsion), catalysts (optional), and the polymerization condition is selected from heating, lighting, etc. The feeding monomer amount relative to initiator amount and/or the feeding monomer amount relative to crosslinker amount and/or the overall monomer concentration is varied to obtain crosslinked hydrogel particles. The obtained zwitterionic hydrogel nanoparticles and zwitterionic hydrogel microparticles are typically purified by centrifugation to remove large gel particles, and dialyzing against water to remove unreacted reagents and surfactants, if any.

According to aspects, zwitterionic bulk hydrogel are synthesized through free radical polymerization method or living polymerization method. These polymerization methods normally involve initiators, zwitterionic monomers, crosslinkers, catalysts (optional), and the polymerization condition is selected from heating, lighting, etc. The feeding monomer amount relative to initiator amount and/or the feeding monomer amount relative to crosslinker amount and/or the overall monomer concentration is varied to obtain crosslinked bulk hydrogels. Zwitterionic hydrogel nanoparticles and zwitterionic hydrogel microparticles are obtained by physical methods, such as grinding, milling, and/or repeatedly extruding the zwitterionic hydrogel through different gauges of needles.

Embodiments of inventive compositions and methods are illustrated in the following examples. These examples are provided for illustrative purposes and are not considered limitations on the scope of inventive compositions and methods.

### EXAMPLES

### Example 1

Materials and Methods

Materials, polymer preparation and characterization, in vivo protein and cell adhesion study, the rat abdominal wall and cecum defect model, the rat repeated-injury adhesion model, and the rat 70% heptectomy-induced adhesion model are described herein.

Materials

The carboxybetaine acrylamide (CBAA) monomer was prepared as described in Zhang, Z. et al., Langmuir 22, 10072-10077, 2006. Ammonium persulfate (APS), fluorescein isothiocyanate (FITC, isomer I suitable for protein labeling, F7250), 2-aminoethyl methacrylate hydrochloride, rat plasma fibronectin (F0635), phosphate buffered saline (PBS, pH 7.4), and paraformaldehyde were purchased from Sigma-Aldrich. Cyanine7 (Cy7)-NHS ester was purchased from Lumiprobe Corporation. Heat-inactivated fetal bovine serum (FBS) was obtained from Hyclone Laboratories. Dulbecco's Modified Eagle Medium/Nutrient Mixture F-12 (DMEM/F-12) and 0.25% trypsin-EDTA were purchased from Thermo Fisher Scientific. Commercially available Interceed® Absorbable Adhesion Barrier (Johnson & Johnson) was purchased from Medex Supply. The SD rat dermal fibroblasts carrying red fluorescence (TurboFP602 red fluorescent protein) were purchased from Innoprot (Spain).

Preparation and Characterization of Zwitterionic PCBAA Solution.

To synthesize zwitterionic PCBAA, an appropriate amount of CBAA monomer and APS initiator (0.5% relative to the total monomer mass) were dissolved in water at a concentration of 10 wt % and then the polymerization was carried out at 37° C. for 24 hours. The resulting solution product was dialyzed against sterile deionized water for 72 hours using a dialysis bag (MWCO 8000 Da) and then freeze-dried to obtain PCBAA powder. To label PCBAA, 2-aminoethyl methacrylate hydrochloride (5% in molar) was added to form a PCBAA polymer with —NH₂ group. 600 mg of PCBAA with —NH₂ group was dissolved in 10 mL of HEPES buffer (pH 8.2), followed by addition of 10 mg of Cy7-NHS in 2 mL of DMSO and stirring in the dark at room temperature for 48 hours. The resulting solution was dialyzed against sterile deionized water for 72 hours (MWCO 3500 Da) to remove unreacted Cy7-NHS and then freeze-dried to obtain PCBAA-cy7.

The molecular weight (Mw) of the prepared PCBAA was characterized using gel permeation chromatography (GPC) (Waters 1525 pump and Waters 2414 differential refractometer) with phosphate-buffered saline (PBS) as eluent at a flow rate of 1.0 mL/min. The monodisperse poly(ethylene glycol) was used as a standard. PCBAA solution was reconstituted in sterile PBS (pH 7.4) at different concentrations, 10%, 20%, and 30% by weight. Shear rheology of various polymer solutions was studied using a TA Instruments ARG2 rheometer (TA Instruments Inc., New Castle, DL) equipped with a 20 mm diameter parallel plate at 25° C. Dissolution rate of PCBAA solution was monitored by measuring the weight loss over time. 1 mL of PCBAA solution was immersed in 10 mL of PBS and incubated in a shaking bath at 37° C. with 125 rpm. At predetermined time points, the supernatant was removed and the remaining sample was lyophilized and weighed.

In Vivo Protein Adsorption Study.

The fluorescein isothiocyanate-labeled rat plasma fibronectin (FITC-Fn) was prepared as follows: 100 uL of FITC solution (0.18 mg/mL) was added to 1 mL Fibronection solution (1 mg/mL) in the carbonate-bicarbonate basic buffer at pH 9.0 and then incubated for 1 hour at room temperature in the dark. The unbound FITC was removed by dialyzing in sterile PBS (pH 7.4) for 2 hours and then in sterile deionized water for 4 hours (MWCO 3500 Da). After freeze-drying, the lyophilized FITC-Fn powder was reconstituted in sterile PBS at the concentration of 1 mg/mL and stored in the dark before use.

Male Sprague-Dawley (SD) rats (250±20 g) were purchased from Charles River Laboratory. The animal experiments were approved by the Institutional Animal Care and Use Committee at Wayne State University and performed in compliance with the relevant laws and institutional guidelines. All the animals were housed in a climate-controlled room under 12 h/12 h light/dark cycle with food and water ad libitum. Rats were given buprenorphine SR (0.4 mg/kg, SQ) prior to surgery. Surgery anesthesia was conducted by isoflurane inhalation (inducted in a chamber and maintained via a nosecone). Appropriate anesthesia levels were monitored by observing respiration rate, eye response, and response to front toe pinch. After the abdominal skin was shaved and prepped with three alternating scrubs of betadine and 70% alcohol, a single 4-5 cm long incision was opened along the linea alba on the abdominal wall with surgical scissors. A 1.5×2 cm defect (including the parietal peritoneum and ˜1 mm of muscle) on the right lateral abdominal wall was created using a scalpel. For the PCBAA group, 500 uL of PCBAA solution was used to cover the sidewall defect. Then 250 uL of prepared FITC-Fn solution (1 mg/mL) was applied on the PCBAA protected defect. For the control group, the FITC-Fn solution was directly applied onto the sidewall defect. Finally, the peritoneum was closed with 3-0 PDS suture (Ethicon), and the skin was closed with 4-0 Nylon suture (Ethicon), respectively. At 2 hours and 24 hours after the surgery, the rats were euthanized by CO₂ and the FITC-Fn treated abdominal walls were harvested and lightly washed three times with PBS and then photographed with the Carestream In Vivo Xtreme Imaging System (Bruker, 480 nm excitation, 535 nm emission).

In Vivo Fibroblast Adhesion Study.

The rat fibroblasts carrying red fluorescence were cultured in DMEM/F-12 medium containing 10% inactivated FBS at 37° C. under the 5% CO₂ atmosphere. Before the in vivo study, fibroblasts were seeded on the coverslip for 48 hours in vitro culture and observed by a EVOS FL fluorescence microscope (AMG, 530 nm excitation, 593 nm emission) and the Carestream In Vivo Xtreme Imaging System (Bruker, 550 nm excitation, 600 nm emission) to confirm the red fluorescence.

For the in vivo study, similar to the typical surgery described above, rats were prepped with abdomen opened. After the cecum was identified, the serosal surface was gently abraded with dry sterile surgical gauze (about 50 strokes) until visible hemorrhaging was developed. A corresponding 1.5×2 cm peritoneal defect on the right lateral abdominal wall was created using a scalpel. Then the injured cecum was sutured to close the injured abdominal wall by puncturing the surrounding mesentery rather than cecum with 3-0 PDS suture. For the PCBAA group, 1 mL of PCBAA was applied on the injured sites (the damaged cecum surface and the abdominal wall defect) and then 250 uL of fibroblasts carrying red fluorescence (2×10⁵ cell/mL) were seeded on the PCBAA protected defects. Likewise, fibroblasts were directly seeded onto the untreated defects as the control. The peritoneum and the skin were closed as described above. At 2 hours, 1 day and 4 day after the surgery, the rats were euthanized and the injured abdominal walls were harvested for fluorescence photography with the Carestream In Vivo Xtreme Imaging System (Bruker, 550 nm excitation, 600 nm emission).

In Vivo Anti-Adhesion Evaluation in a Rat Abdominal Wall and Cecum Defect Model.

The sidewall defect-cecum abrasion model was established as described in the fibroblasts adhesion study, except that fibroblasts were not applied. For the PCBAA group, 1 mL of PCBAA solution was injected onto the injured abdominal wall and damaged cecum. As a positive control, the defects were also covered by a commercialized 2×2 cm Interceed® film. For negative control, no anti-adhesion material was employed on the injured area. 1 week and 2 weeks after the surgery, 6 rats for each group were euthanized by CO₂ asphyxiation. The peritoneum was opened and the extent of adhesion was evaluated and scored based on the standard adhesion scoring system as follows: score 0, no adhesion; score 1, mild, easily separable intestinal adhesion; score 2, moderate intestinal adhesion, separable by blunt dissection; and score 3, severe intestinal adhesion requiring sharp dissection to separate. Tissues collected from different groups were fixed in 4% paraformaldehyde, embedded in paraffin, sectioned, and stained with Hematoxylin and Eosin (H&E) and Masson trichrome, and then imaged using an EVOS XL Core microscope (Thermo Fisher Scientific).

In Vivo Anti-Adhesion Evaluation in a Rat Repeated-Injury Adhesion Model.

The repeated-injury adhesion model was established by creating a first abdominal wall and cecum injury with the peritoneum closed without any anti-adhesion material treatment as untreated control described in the sidewall defect-cecum abrasion model. One week later, a second laparotomy was performed to create a repeated injury. After reopening the incision, the adhesion site resulted from the first surgery was separated by an appropriate dissection as needed, and then the separated abdominal wall and cecum were abraded monodirectionally with a sterilized brush until bleeding surfaces were produced. For treatment groups, the repeated-injured sites were covered either by 1 mL of PCBAA solution or 2×2 cm Interceed® film before the final closure. For the untreated control group, no anti-adhesion material was employed on the repeated-injured sites. At 7 and 14 days after the second surgery, 6 rats for each group were euthanized by CO₂ asphyxiation. The peritoneum was opened, and the extent of adhesion was evaluated and scored. The tissues were collected for histological examination.

In Vivo Anti-Adhesion Evaluation in a Rat 70% Hepatectomy-Induced Adhesion Model.

Similar to the typical surgery described above, rats were prepped with abdomen opened. Hepatectomy was performed by ligating the pedicle of the median lobe using 3-0 silk sutures, then cutting the parenchyma of median lobe using surgical scissors near the base of the lobe. Similarly, the left lateral lobe was ligated and resected. After lavaging the peritoneal cavity with 15 mL of sterile saline, the cut surfaces of the excisional liver parenchyma were completely covered with 2 mL of injected PCBAA. For the film treated group, the 2×2 cm Interceed® film was applied to cover the cut surfaces and the surface of remnant liver lobes. For the untreated control group, the peritoneal cavity was closed without any anti-adhesion materials applied. After surgery, 20% glucose water was provided instead of regular water for 3 days. Day 7, 14, and 30 after the hepatectomy, 6 rats in each group were euthanized and the abdomen was opened to evaluate and score the adhesions. The presented adhesions at the cut surface, diaphragm, hepatic hilum, and remnant liver surface were scored respectively since the locations of adhesion formation were uncertain in this model.

Statistical Analysis.

All data were presented as mean±SD and statistical analysis was performed using GraphPad Prism software (GraphPad Software). Adhesion scores did not always follow a normal distribution, so statistical analysis was performed using a non-parametric Mann-Whitney U test. The body weight data were normally distributed and analyzed by one-way ANOVA followed by Tukey multi-comparison tests. For all statistical analyses, significance was accepted at the 95% confidence level, and all analyses were two-tailed. Statistical differences were defined as *P<0.05, **P<0.01, and differences with P<0.05 were considered significant.

Characterization of Zwitterionic PCBAA.

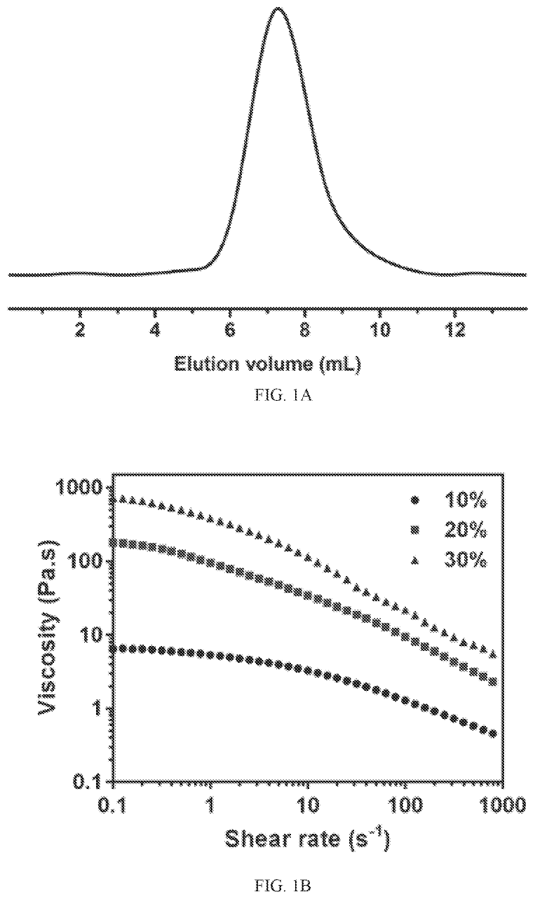

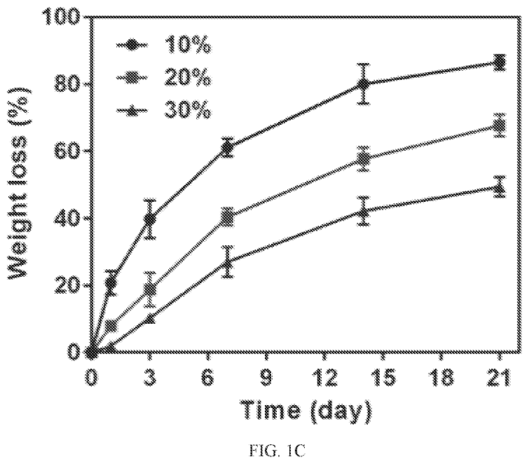

The molecular weight (MW) of the prepared zwitterionic PCBAA was 42 kDa (FIG. 1A) as characterized using gel permeation chromatography (GPC). The low MW is expected to enable an ultimate removal of the polymer from systemic circulation after in vivo application (below the MW cut-off for glomerular filtration-70 kDa). In a shear rheology study, PCBAA solutions at different concentrations showed marked shear-thinning behavior with reducing viscosity by at least one order of magnitude over shear rates extending from 0.1-1000/s, see FIG. 1B. In vitro dissolution test indicated that PCBAA solutions were gradually dissolved over time, see FIG. 1C). 10 wt % PCBAA solution had more than 60% dissolved on day 7 and almost totally dissolved on day 21. By contrast 30 wt % PCBAA solution showed no observable dissolution on day 7. Therefore, 20 wt % PCBAA solution was selected for the in vivo adhesion prevention study, which showed a medium viscosity (cream-like yet injectable) and dissolution rate, see FIGS. 1A-1C.

Zwitterionic PCBAA Resisted Fibronectin Adsorption on Rat Abdominal Wall Wound In Vivo.

After surgical trauma to the peritoneal surface, deposited matrix (from exudate and body fluid) acts as a docking site for subsequent inflammatory cells and fibroblasts, facilitating the connection between damaged intra-abdominal surfaces. Among the various components of the deposited matrix, fibronectin is critical and can direct cellular adhesion and migration (mainly macrophages and fibroblasts) at initial-stage in the formation of postoperative adhesions. Resisting fibronectin adsorption on the traumatized surface in vivo can reduce the fibroblast adhesion and subsequent permanent tissue adhesion formation.

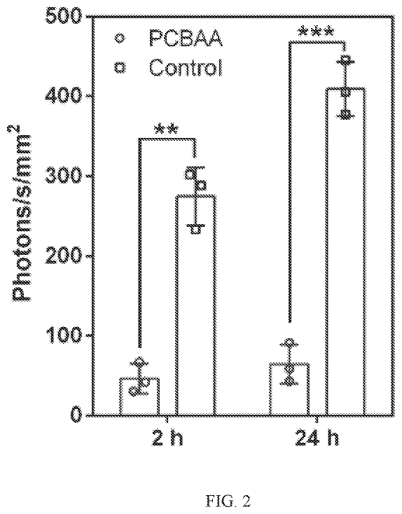

To evaluate the efficacy of zwitterionic PCBAA in resisting fibronectin adsorption on the injured rat abdominal wall in vivo, a peritoneal defect was created on the right lateral abdominal wall, followed by injecting 500 uL of the cream-like zwitterionic PCBAA solution (20 wt %) onto the injured abdominal wall. Then 250 uL of prepared fluorescein isothiocyanate-labeled fibronectin (FITC-Fn) solution (1 mg/mL, mimicking the one from body fluid) was applied on the PCBAA protected injured site. For the control group, the FITC-Fn solution was directly applied onto the defect. By 2 h and 24 h after the surgery, the FITC-Fn treated abdominal walls were harvested and lightly washed three times with sterilized PBS and then photographed with the Carestream In Vivo Xtreme Imaging System (Bruker). There was no significant difference in the appearance of the abdominal wall defects between the two groups on gross observation. Nevertheless, fluorescence photography clearly showed that almost no fluorescence (no fibronectin absorption) on the PCBAA protected trauma at either 2 hours or 24 hours. By contrast, the untreated group showed significant signal indicating fibronectin absorption on the defect with fluorescent intensity increasing over time after the surgery (see FIG. 2). These results show that the zwitterionic PCBAA effectively resisted fibronectin adsorption on the surgical trauma after abdominal surgery.

Zwitterionic PCBAA Resisted Fibroblast Adhesion on Rat Abdominal Wall and Cecum Wound Model In Vivo.

Fibroblast adhesion and proliferation on the deposited matrix play a key role in the late-stage adhesion formation. Once invaded by fibroblasts, the initial fibrin matrix is gradually replaced by deposited collagen, leading to the formation of permanent adhesion. Therefore, prevention of fibroblasts invasion and adhesion is a key step for anti-adhesion and was evaluated.

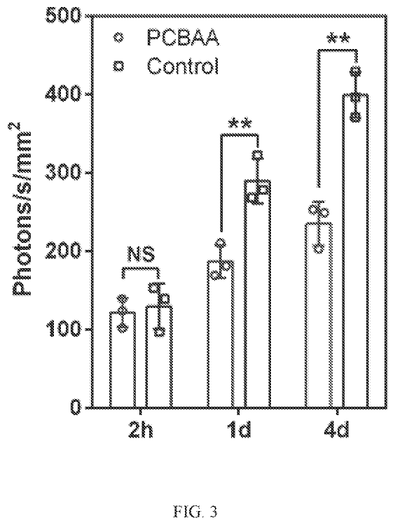

SD rat dermal fibroblasts carrying red fluorescence (TurboFP602 red fluorescent protein) were purchased with their fluorescence verified and calibrated before the in vivo study, using both EVOS FL fluorescence microscope (AMG) and the Carestream In Vivo Xtreme Imaging System (Bruker). The rat abdominal wall and cecum wound model was created by introducing two defects through abrasion of the cecum and partial abdominal wall excisions, followed by injecting 500 uL of PCBAA solution on each of the defect. 250 uL of fibroblasts (2×10⁵ cell/mL) carrying red fluorescence were then seeded on the PCBAA protected injured sites. For the control group, the fibroblasts were directly seeded onto the defect. 2 hours after the surgery, no fluorescence (no fibroblasts adhesion) was observed on the PCBAA protected and unprotected defects. 1 and 4 days after the surgery, significantly more and more intensive fluorescence, representative of fibroblast adhesion, was shown on the unprotected defects, but only a limited increase in fluorescence was observed on the PCBAA protected surface (see FIG. 3). These results indicate that applying zwitterionic PCBAA on the traumatized surface prevented fibroblast invasion and adhesion for at least four days in vivo after the surgery, and thereby reduced collagen secretion and deposition and subsequent tissue adhesion formation.

Zwitterionic PCBAA Prevented Postoperative Adhesion in a Rat Abdominal Wall-Cecum Defect Model.

The in vivo anti-adhesion efficacy of PCBAA was first evaluated in a rat model of sidewall defect-cecum abrasion, where the defects were created by abrasion of the cecum and partial abdominal wall excisions. To apply the anti-adhesion formulation, 1 mL of PCBAA solution was injected onto the injured abdominal wall and damaged cecum. As a positive control, the defects were covered by the commercial Interceed® film (Johnson & Johnson). As a negative control, no anti-adhesion material was applied to the injured area in control animals.

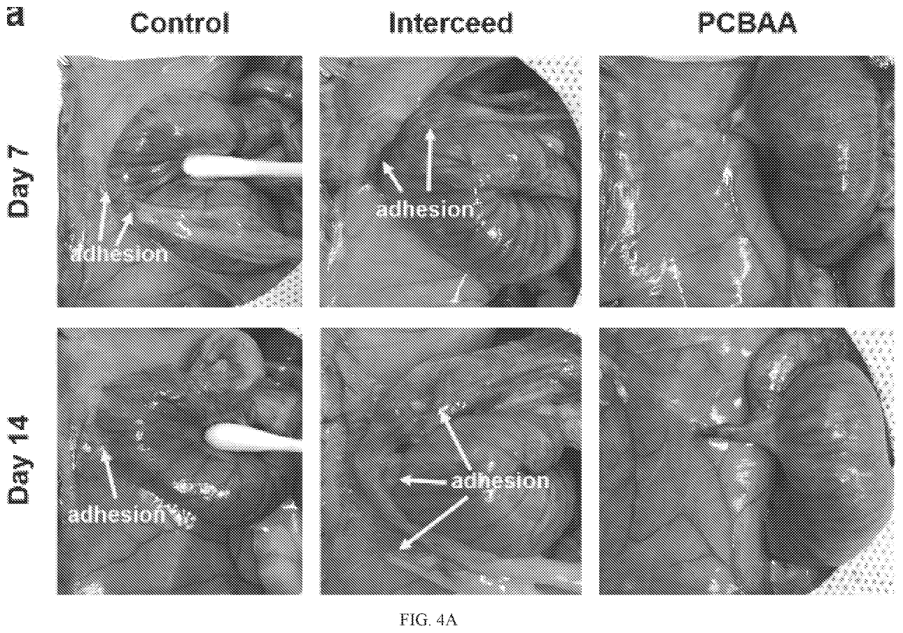

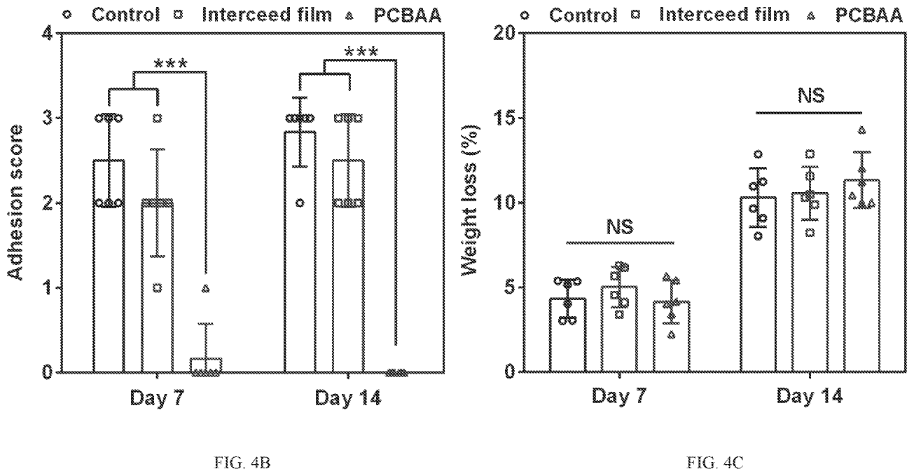

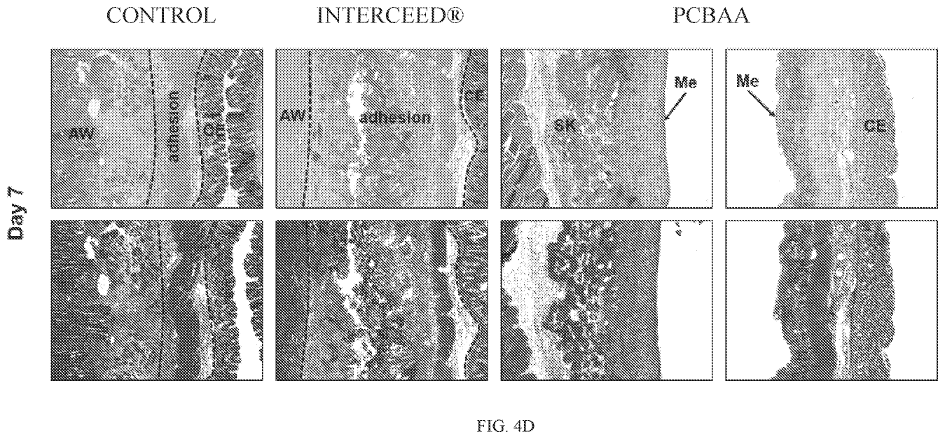

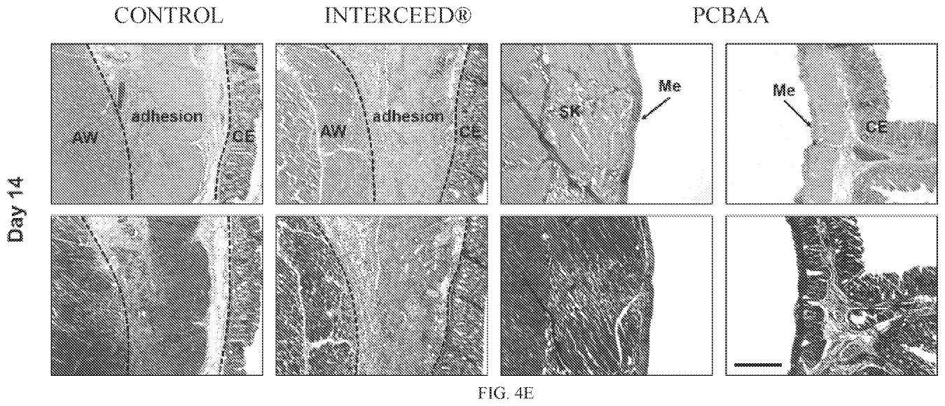

At 7 and 14 days after the surgery, the peritoneum was opened and the extent of adhesion was evaluated, see FIG. 4A, and scored, see FIG. 4B. In the control group which received no treatment, all rats (n=6) suffered from severe abdominal adhesions scored at 2 and 3 at day 7, and most developed more serious and tenacious adhesions with score 3 at day 14. For the group treated with Interceed® films, rats still suffered from peritoneal adhesions at day 7, but both the adhesion scores and adhesion area were significantly reduced (mostly score 2) compared with the untreated group, indicating the film could alleviate the postoperative adhesion to some extent. At day 14, adhesions in the film group became worse, which were not only visible in injured sites but also involved the circumjacent mesentery. It should be noted that the postoperative adhesions in the untreated and the film groups occurred not only between the injured cecum and abdominal wall but also between the abrased cecum and the proximal mesentery in some cases. By comparison, for PCBAA treated group, nearly no adhesion was observed on day 7 and both the damaged abdominal wall and cecum were partly healed. At day 14, 100% no adhesion was observed at all (score 0), and both the injured abdominal wall and cecum were completely healed; this indicates that PCBAA fully prevented postsurgical peritoneal adhesions in this model. In addition, no apparent PCBAA residue was present on the treated sites or abdominal cavity at day 14 post-surgery, indicating that the material was able to be absorbed. The change of body weight after the surgery did not show significant difference among these groups (p >0.05), see FIG. 4C.

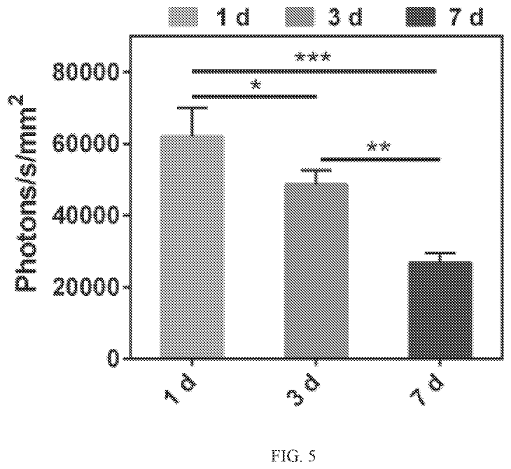

To further evaluate the retention of PCBAA presented on the treated sites, PCBAA was labeled with Cy7 and the applied PCBAA-Cy7 was examined both visually and through fluorescent quantification in the rat sidewall defect-cecum abrasion adhesion model, see FIG. 5. 1 day and 3 days after the application, a significant amount of polymer remained at the traumatized site. 7 days after the application, only a minimum amount of the polymer remained. Fibroblast invasion and adhesion within the first 3 days after the surgical injury is a key step for tissue adhesion formation. These data indicated that PCBAA remains at the application site during the critical days and beyond, preventing fibroblast invasion and adhesion.

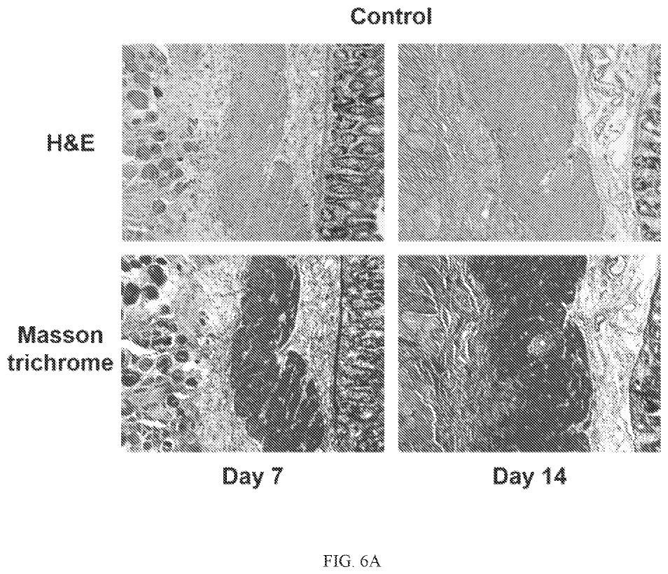

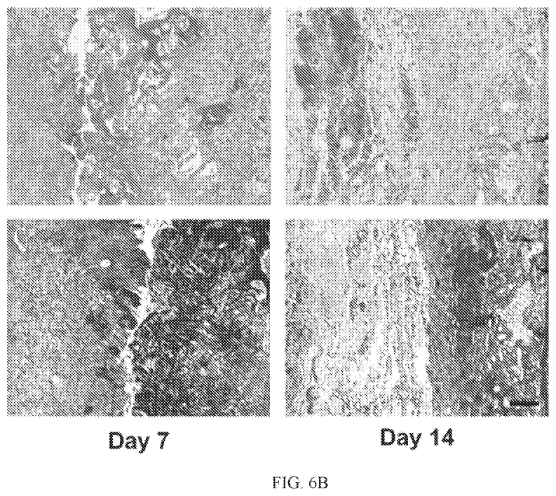

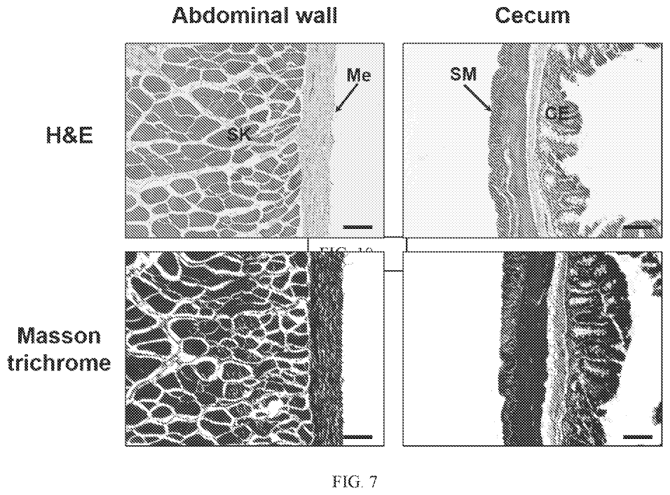

Tissue samples were collected from different groups and histological analysis using Hematoxylin and Eosin (H&E) and Masson trichrome staining were performed, see FIGS. 4D and 4E. For untreated group on day 7, the skeletal muscles of the injured abdominal wall and the smooth muscles of injured cecum were fused with connective tissues (adhesive region), which composed of granulation tissue, collagen deposition, and fibroblasts and some inflammatory cells, see FIG. 6A. Deposited collagen at the adhesion site can be easily observed in Masson Trichrome staining with intense blue. The film group on day 7 presented a similar adhesive structure as the untreated control group with looser adhesion region and less collagen deposition (see FIG. 6B). On day 14, both untreated and film-treated groups showed more compacted adhesion region with increased thickness and more granulation tissue and collagen deposition. In addition, many new blood vessels could be observed in the adhesion site, see FIGS. 6A and 6B, indicating the formation of mature adhesions. For the PCBAA treated group, histological observations of the injured abdominal wall and cecum were performed separately since no adhesion was developed at all. On day 7, it was clearly observed that the injured abdominal wall (skeletal muscle was shown) and cecum were healed with a comparatively complete neo-mesothelial cell layer at the damaged sites. On day 14, the damaged abdominal wall and cecum have completely re-mesothelialized with a smooth and fully developed mesothelium layer similar to that in the normal tissue, see FIG. 7. These results indicate that the PCBAA completely and reliably prevented the formation of postoperative adhesion in the abdominal wall defect-cecum abrasion adhesion model without interfering regular wound healing.

Zwitterionic PCBAA Prevented Recurrent Adhesion after Adhesiolysis in a Rat Repeated-Injury Adhesion Model.

In clinical practice, adhesiolysis is an indispensable operation for patients to eliminate pre-existing postoperative adhesions. Unfortunately, the new trauma resulted from the surgical lysis of the prior adhesions tends to induce recurrent adhesion. Although minimally invasive procedures such as laparoscopy have been used in adhesiolysis to decrease the peritoneal trauma and thus prevent new adhesion formation, there is still a high incidence (more than 55%) of recurrent adhesion regardless of adhesiolysis being performed by laparoscopy or laparotomy. As a matter of fact, the prevention of recurrent adhesion after adhesiolysis is more difficult because the trauma is more severe and the adhesion mechanism is more complicated when compared with the primary adhesion.

Thus, the efficacy of the PCBAA on the prevention of recurrent adhesion after adhesiolysis with a more rigorous rat repeated-injury adhesion model was investigated.

The repeated-injury adhesion model was established by creating a first abdominal wall and cecum injury with the peritoneum closed without any anti-adhesion material treatment (on day −7), followed by a second surgery on day 0 to reopen the incision, lyse the adhesion site that resulted from the first surgery by an appropriate dissection as needed, and abrade the separated abdominal wall and cecum monodirectionally with a sterilized brush until bleeding surfaces were produced. For treatment groups, the repeated-injured sites were covered either by 1 mL PCBAA solution or Interceed® film before the final closure.

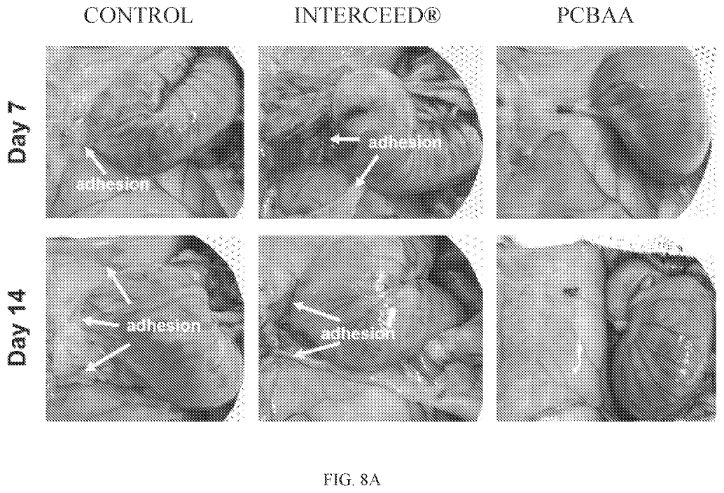

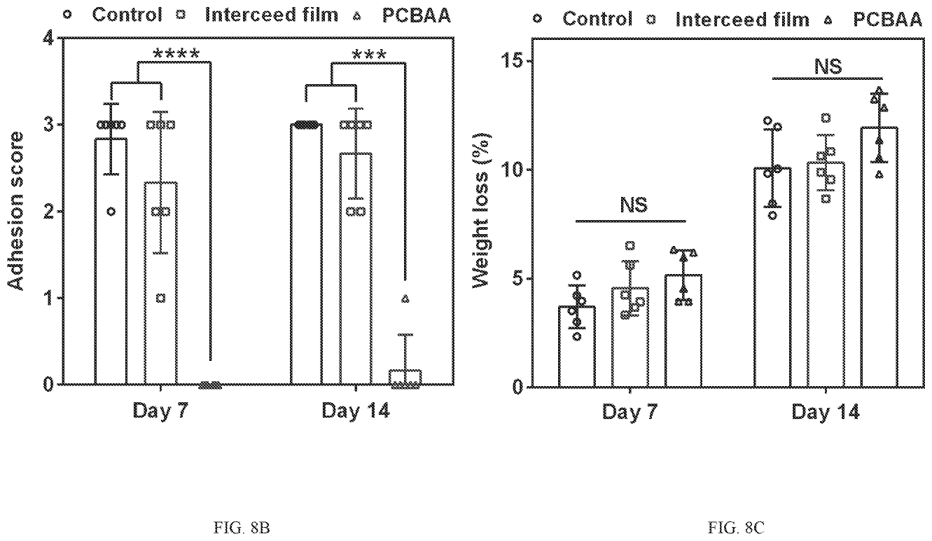

7 and 14 days after the second surgery, the rats were euthanized and the extent of recurrent adhesions was evaluated, see FIG. 8A, and scored, see FIG. 8B. On day 7 for the control group receiving no treatment, 5 out of 6 rats suffered from severe recurrent adhesions scored at 3 and the remaining rat had severe recurrent adhesions scored at 2. The adhesion area was observed to be larger than the initial injured area, indicating the uninjured surface of abdominal wall and cecum were also involved in the recurrent adhesion. On day 14, all 6 rats in the untreated group developed more serious vascularized adhesions scored at 3. The adhesions were not only visible between the injured sites but also involved the uninjured surface and circumambient mesentery. For the group treated with Interceed® films, rats still suffered the recurrent adhesions at day 7 and 14 similar to the untreated group, but both the adhesion scores and adhesion area were reduced. By contrast, in the PCBAA-treated animals on day 7, 100% no adhesion was observed (score 0) in any of the rats and the re-injured abdominal wall was mostly healed (the wound area was still visible). On day 14, nearly no adhesion was observed and the damaged abdominal wall and cecum were completely healed with visible new blood vessels on the smooth surface. These results indicate that the administered PCBAA prevents recurrent postsurgical peritoneal adhesions as demonstrated in this model which mimics the complications after adhesiolysis.

Additionally, on day 14 post-the-second-surgery, no apparent PCBAA residue was present on the treated sites or abdominal cavity, indicating that the material was able to be absorbed. No significant difference in body weight was measured among these groups (p >0.05), see FIG. 8C. Rats treated with PCBAA were closely monitored and recorded after the second surgery to evaluate potential signs for toxicity. All animals displayed normal behavior without any adverse reactions such as slow movement, eye secretion, abnormal food and water intake.

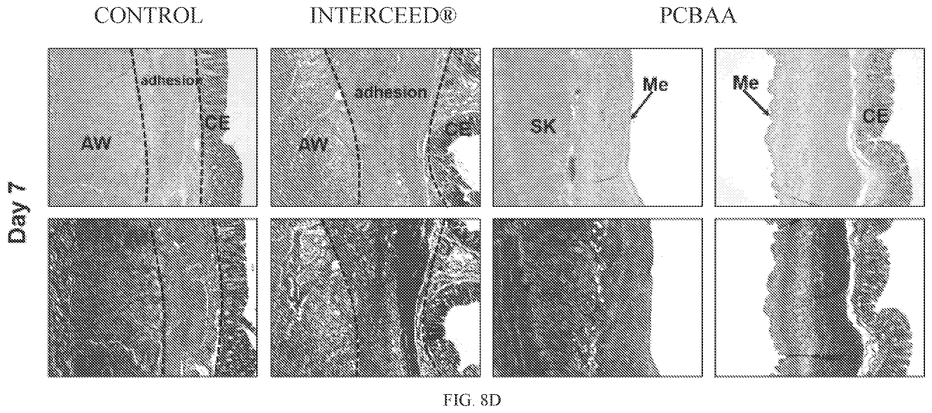

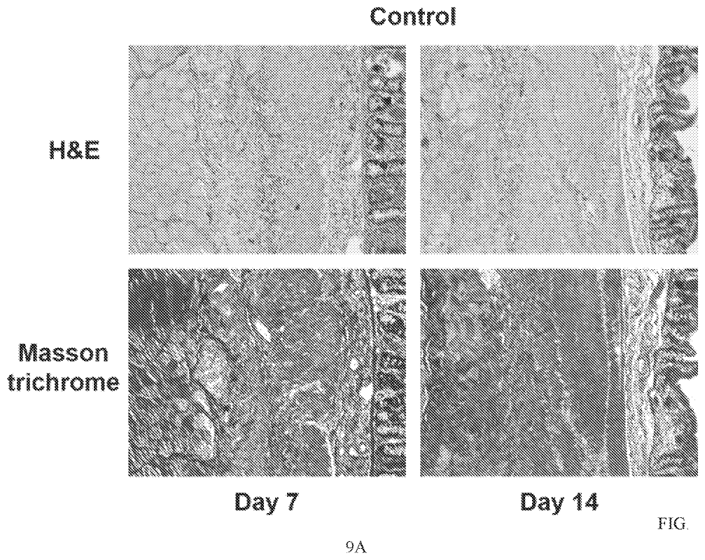

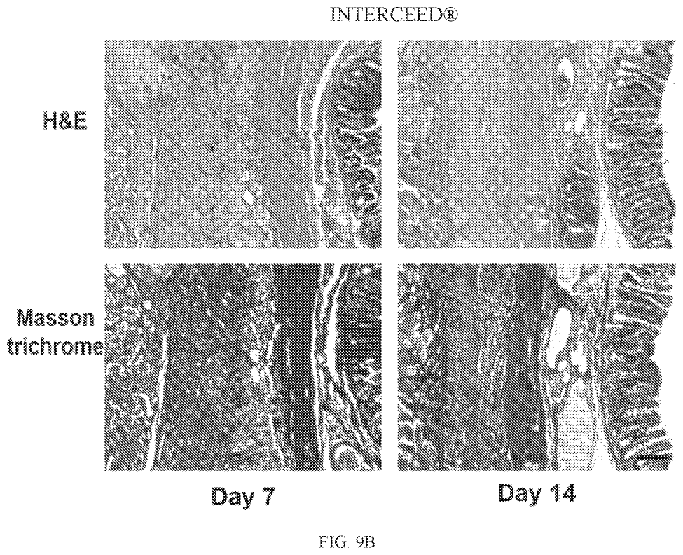

Tissue samples from different groups were collected to perform histological analysis by H&E and Masson trichrome staining, see FIG. 8D. On day 7 after the second surgery, the tissues taken from the adhesion site in the control and film groups showed that the injured cecum was fused to the skeletal muscles of damaged abdominal wall, and the resulting adhesion contained a great deal of granulation tissue, fibroblasts, inflammatory cells, and deposited collagen (staining with intense blue in Masson Trichrome), see FIGS. 9A and 9B. For the film treated group, some residual film was observed in the adhesion region. On day 14, both untreated and film-treated groups showed mature adhesion with neovascularization and more granulation tissue and collagen deposition, see FIGS. 9A and 9B. For the animals treated with PCBAA, both the injured abdominal wall and cecum were recovered with an integral neo-mesothelial cell layer despite some fibroblasts, inflammatory cells, and collagen populated at the damaged sites on day 7. Two weeks later, the surfaces of the healed abdominal wall and cecum were completely remesothelialized with decreasing collagen deposition. These results showed that PCBAA fully prevented the recurrent adhesion in the rigorous rat repeated-injury adhesion model.

Zwitterionic PCBAA Prevented Postoperative Adhesion in a Rat 70% Hepatectomy-Induced Adhesion Model

Liver cancer is the fifth most prevalent human malignancy while the second most frequent cause of cancer-related death. Despite the progress in cancer treatment over the last 50 years, liver resection remains the most available and effective therapy for patients with hepatocellular carcinoma, and currently represents the only potentially curative therapy for liver metastases from colorectal cancers. Nevertheless, intraabdominal adhesions induced by hepatectomies posed significant clinical challenges; they not only brought suffering and postoperative complications to the patients but also created additional troubles in reoperative procedures, such as longer operation time and a higher risk of bleeding or injury to adhered organs. In particular, numerous patients required repeated liver resections in clinical practice due to the frequent recurrent liver cancer after a first liver resection, whereas the presence of adhesions around the remaining liver from the previous hepatectomy significantly impeded the feasibility of repeat liver resection due to increased technical difficulties.

A classical rat partial hepatectomy model, removing approximately 70% of the total liver, was used to study hepatectomy-induced peritoneal adhesion. This model presents several distinctive features compared with commonly reported adhesion models: 1) the median lobe (ML) and the left lateral lobe (LLL) of the liver, 70% of the total liver, are resected, which is a more standardized injury than previous abrasion models, 2) this model creates a complex three-dimensional (3D) cut surface, which is harder to completely cover than sidewall defect models, 3) this model can reproduce more severe adhesions compared with other animal models since the hepatectomy consistently causes severe trauma to the peritoneal cavity, and 4) the locations of adhesion formation are uncertain because the injured surface is in contact with several surrounding tissues, including hepatic hilum, diaphragm, remnant liver, small bowel, and omentum, etc. Therefore this standardized and reproducible model with severe adhesions can serve as a powerful tool to evaluate and compare the efficacy of various anti-adhesive materials for adhesion prevention.

The SD rat liver is composed of four main lobes, including the left lateral lobe (LLL), median lobe (ML), right liver lobe (RLL), and the caudate lobe (CL), among which ML and LLL account for approximately 70% of the total liver. To establish the 70% hepatectomy model, the ML and LLL, were resected after ligating the hepatic pedicle through a midline incision. For the untreated control group, the peritoneal cavity was closed without any anti-adhesion materials. For PCBAA group, the complex cut surface was completely covered with 2 mL of injected PCBAA. For the film treated group, the Interceed® film was applied to cover the cut surfaces and the surface of remnant liver lobes. Nevertheless, full coverage of the cut surfaces with this film product was difficult since the cut surfaces presented highly complicated 3D structure.

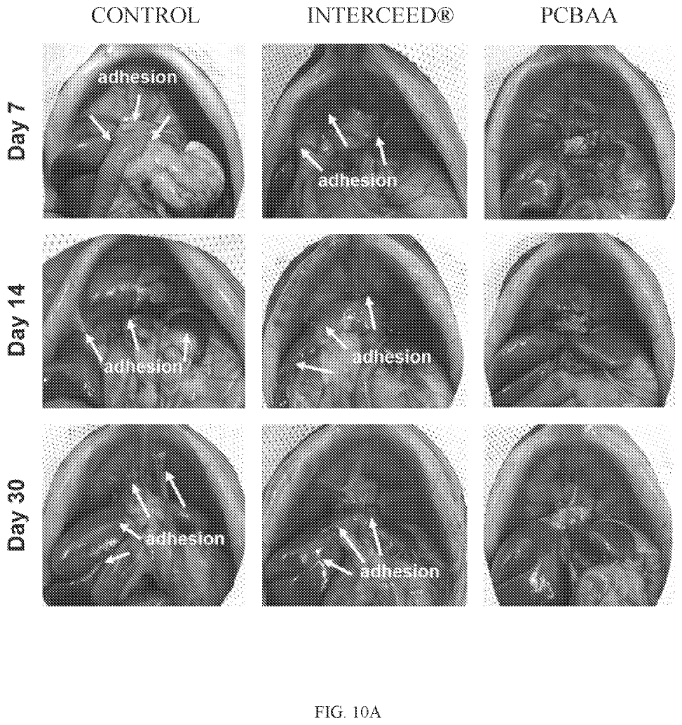

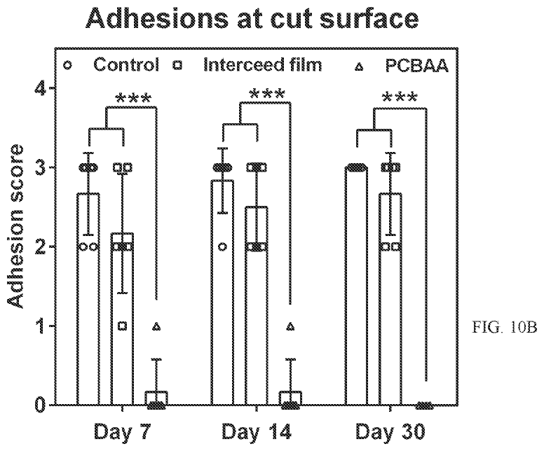

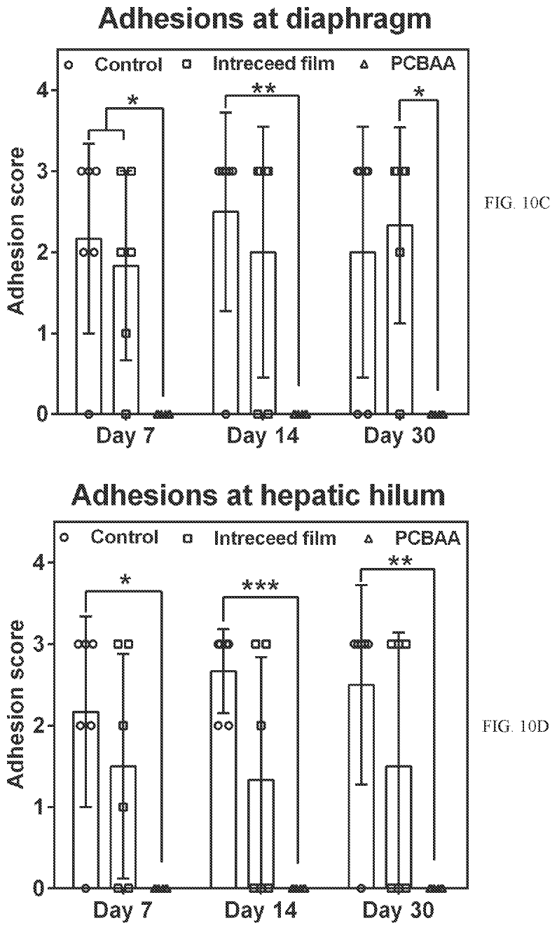

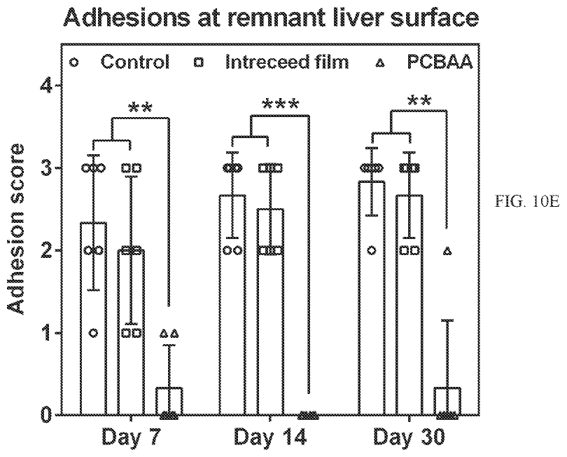

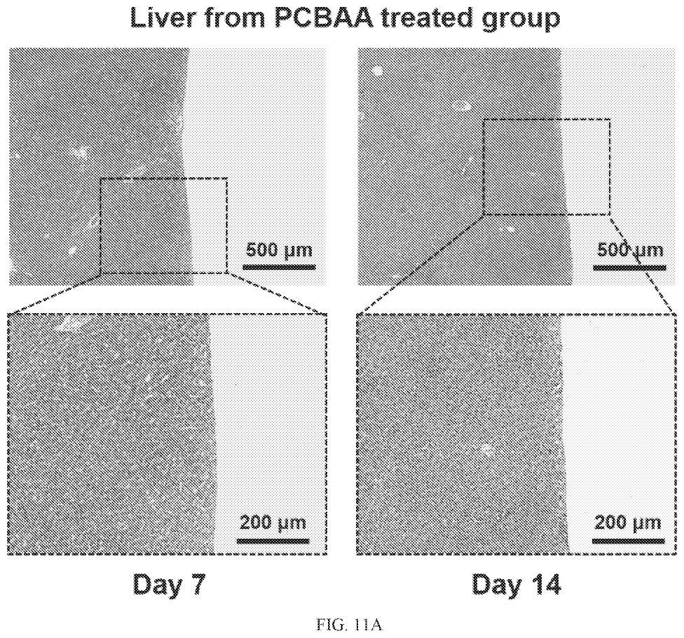

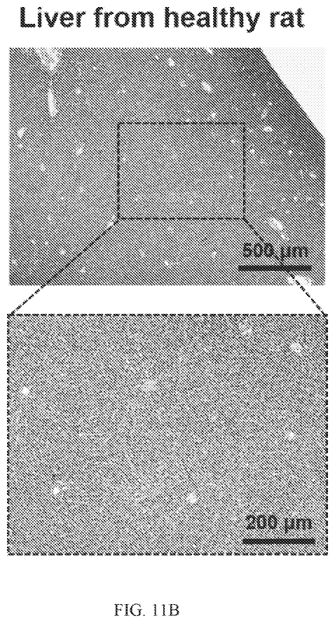

One week after the hepatectomy, 6 rats in each group were euthanized and the abdomen was opened to evaluate and score the adhesions. All the rats in the untreated group developed severe adhesions, see FIG. 10A. The location of the adhesions was not limited to the cut surface and adjacent organs (mainly small bowel or omentum), but also included the diaphragm, hepatic hilum and remnant liver surface, adhesion scored respectively in FIGS. 10B, 10C, 10D, 10E. The Interceed® film treated rats showed slightly lower adhesion scores compared to the untreated one. Most rats developed adhesions between the cut surface of the liver and the omentum, rather than small bowel that presented in the control group. Meanwhile, the adhesions were also observed between the diaphragm and the omentum, see FIG. 10A. Of note, the adhesions around the cut surface were relatively easier to separate than that in the untreated control group, indicating the film could alleviate the postoperative adhesion to some extent. For PCBAA treated group, hardly any adhesion was observed in this highly challenging adhesion model. No apparent PCBAA residue was observed in the abdominal cavity on day 7 after the hepatectomy and the cut surface with small portion of the residual liver parenchyma could be clearly observed without any adhesion. On day 14 and 30 after the hepatectomy, all the animals in control and film group developed more mature and vascularized adhesions that were hard to separate. By contrast, the PCBAA group did not suffer from adhesion except for two rats that developed slight adhesion between the superior right lobe and the inferior right lobe. In addition, it was clearly observed that the remnant right liver lobe and posterior caudate lobe showed an obvious increase in volume compared to day 7-an expected compensatory liver regeneration after partial hepatectomy. In addition, histological examinations showed that the increscent remnant lobes were similar to that in the healthy rat, see FIGS. 11A and 11B, without any abnormality further demonstrating that PCBAA effectively prevents postoperative adhesions after partial hepatectomy.

These results show that the injectable zwitterionic PCBAA polymer can completely and reliably prevent postoperative adhesions under three clinically relevant and increasingly challenging models. The PCBAA formulation fully prevents protein adsorption, such as fibronectin absorption, on a wound surface in vivo administered within the 24 hours of surgery, i.e. during the initial stage development of adhesions, and remarkably reduce cell invasion, such as fibroblast invasion, and adhesion in vivo by day four after the surgery (the late-stage development of the adhesion).

### Example 2

Materials and Methods

Materials

The carboxybetaine acrylamide (CBAA) monomer was prepared as described in W. Wang, Y. Lu, H. Zhu, Z. Cao, Adv. Mater. 2017, 29, 1606506. N, N′-bis(acryloyl)cystamine (BAC), ammonium persulfate (APS), glutathione (GSH), rat plasma fibronectin (F0635), phosphate buffered saline (PBS, pH 7.4), Hanks' balanced salt solution (HBSS), and paraformaldehyde were purchased from Sigma-Aldrich. Heat-inactivated fetal bovine serum (FBS) was obtained from Hyclone Laboratories. Dulbecco's Modified Eagle Medium (DMEM), 0.25% trypsin-EDTA, and Live/Dead viability/cytotoxicity kit were purchased from Thermo Fisher Scientific. Commercially available Interceed® Absorbable Adhesion Barrier (Johnson & Johnson) was obtained from Medex Supply. National Institutes of Health 3T3 (NIH/3T3) mouse embryonic fibroblast cells were purchased from the American Type Culture Collection without further authentication.

Preparation of Zwitterionic PCBAA Bulk Gel and Cream Gel

N,N′-bis(acryloyl)cystamine (BAC) crosslinker (0.1 or 1% relative to monomer by weight) and APS initiator (0.2% relative to monomer by weight) were added to the 40 wt % CBAA monomer solution, then the resulting solution was purified by a 0.22-μm filter. After thorough mixing and degassing, the solution was stored at a vial to prepare the bulk hydrogel at 37° C. for 24 hours or transferred into two glass slides with a 0.1 mm polytetrafluoroethylene spacer to prepare hydrogel disk, followed by equilibration in sterile PBS for 3 days. The bulk gel was further repeatedly extruded through sterile 18G, 20G, 22G, and 26G needles to obtain cream gel.

Rheological Measurements

Rheological characterization was performed using a ARG2 rheometer (TA Instruments Inc., New Castle, DL) equipped with 20 mm or 40 mm diameter parallel plates at 25° C. For oscillatory frequency sweep test, a solvent trap bar was loaded to avoid evaporation. All results were recorded and analyzed using TA Instruments TA Orchestrator software.

In Vitro Degradation of PCBAA Cream Gel

In vitro degradation of PCBAA cream gel was monitored by measuring weight loss over time. 1 mL of cream gel was lyophilized and recorded as the initial weight. Then 1 mL of cream gel was immersed in 10 mL of PBS (10 mM, pH 7.4) containing 20 μM GSH and incubated in a shaking bath at 37° C. at 125 rpm. The degradation media were refreshed daily to ensure the GSH concentration. At predetermined time points, the cream gels were lyophilized and weighed.

In Vitro Cytotoxicity and Live Dead Assay

In vitro cytotoxicity was evaluated by the 3-(4,5-dimethylthiazol-2-yl)-2,5-diphenyltetrazolium bromide (MTT) assay using National Institutes of Health (NIH) 3T3 mouse embryonic fibroblast cells. 3T3 cells were seeded on 96-well plates (2×10⁴ cells/well) in DMEM with 10% FBS and cultured in 5% CO₂ atmosphere at 37° C. for 24 hours. Then culture media were substituted by DMEM containing PCBAA cream gel extraction or degradation products for another 24-hour incubation. The cream gel extraction was obtained by incubating 1 mL of cream gel in DMEM with 10% FBS for 48 hours at 37° C. The media containing degradation product was prepared by incubating 1 mL of cream gel in DMEM with 20 mM GSH for 48 hours at 37° C. After the challenge, the cell media were replaced with 150 μL of HBSS containing MTT (0.5 mg/mL), followed by a further incubation at 37° C. for 4 hours. Subsequently, the supernatant was replaced by dimethyl sulfoxide (150 μL/well). The absorbance was tested by microplate reader (Bio-Rad) at 570 nm. The cells cultured with regular DMEM medium were used as a negative control for 100% cell viability. Live/dead assay was conducted by staining the 3T3 cells with 150 μl of PBS buffer containing 2 μM Calcein and 4 μM ethidiumhomodimer-1 (Live/Dead viability/cytotoxicity kit, Invitrogen) for 30 min at room temperature and observed under a EVOS FL fluorescence microscope (AMG).

Fibronectin Adsorption Assay

The fibronectin adsorption on disk and cream gel was evaluated by a standard bicinchoninic acid (BCA) method as follows. After being equilibrated with PBS for 3 days, PCBAA hydrogel disk (12 mm in diameter and 0.2 mm in thickness) or 100 μL of PCBAA cream gels were immersed in 0.5 mL of fibronectin solution (50 ug/mL) at 37° C. for 2 hours. Then fibronectin solution was removed and the cream gels were carefully rinsed with fresh PBS three times. The cream gels were then soaked in 0.5 mL of 1% sodium dodecyl sulfate (SDS) for 1 hour to detach the adsorbed protein. The concentration of the adsorbed protein was determined using a micro-BCA™ protein assay reagent kit by microplate reader at 562 nm.

Cell Adhesion Assay

2×10⁴ of 3T3 cells were seeded on the surface of a PCBAA hydrogel disk (12 mm in diameter and 0.2 mm in thickness) and incubated at 37° C. in a 5% CO₂ atmosphere for 48 hours. Then cell media were removed and the hydrogel disk was gently rinsed with HBSS to remove unattached cells. 3T3 cells attached on the surface were stained by Live/Dead kit and the morphology was observed by the EVOS FL fluorescence microscope (AMG). 3T3 cells seeded in the tissue culture plate (TCP) were used as a control.

In Vivo Anti-Adhesion Evaluation in a Rat Abdominal Wall and Cecum Defect Model

Sprague-Dawley rats (Male, 250±20 g, purchased from Charles River Laboratory) received buprenorphine SR (0.4 mg/kg, SQ) prior to surgery. Under an appropriate anesthesia (isoflurane inhalation), the abdominal skin was shaved and prepped with three alternating scrubs of betadine and 70% alcohol, followed by a single incision (4-5 cm long) along the linea alba on the abdominal wall using surgical scissors. The serosal surface of the cecum was gently abraded using a dry surgical gauze (50 strokes) to result in a visible hemorrhaging. A peritoneal defect on the right lateral abdominal wall (1.5×2 cm; including the parietal peritoneum and approximately 1 mm of muscle) was further created using a scalpel. The injured cecum and abdominal wall were forced to contact each together by suturing the surrounding mesentery with 3-0 PDS suture. For anti-adhesion treatment, cream gel was injected onto the injured abdominal wall and cecum without hemostasis. For the film treatment group, a commercial Interceed® film was used to cover the defects. 7 and 14 days after the surgery, rats were euthanized to open the peritoneum and examine the adhesion level. A standard adhesion scoring system was used: score 0, no adhesion; score 1, mild, easily separable intestinal adhesion; score 2, moderate intestinal adhesion, separable by blunt dissection; and score 3, severe intestinal adhesion requiring sharp dissection to separate. Cecum and abdominal wall tissues relating to the injury and/or adhesion were collected and processed for histological evaluation (hematoxylin and eosin (H&E) and Masson trichrome) under an EVOS XL Core microscope (Thermo Fisher Scientific).

In Vivo Anti-Adhesion Evaluation in a Rat Repeated-Injury Adhesion Model

The repeated-injury adhesion model was established first by creating a primary adhesion as described in the sidewall defect-cecum abrasion model without applying any anti-adhesion material. 7 days later, a second laparotomy was performed, and the primary adhesion was separated by an appropriate dissection, followed by abrading the separated abdominal wall and cecum monodirectionally with a sterilized brush to produce a bleeding surface. For anti-adhesion treatment, cream gel or Interceed® film was similarly applied as described in the prior model. 7 and 14 days after the surgery, rats were subject to adhesion scoring and histological evaluation as described in the prior model.

Statistical Analysis

All data were presented as mean±s.d. and statistical analysis was performed using GraphPad Prism software. Adhesion scores did not always follow a normal distribution, so statistical analysis was performed using a non-parametric Mann-Whitney U test. The data of body weight were normal distributed and analyzed by one-way ANOVA followed by Tukey multi-comparison tests. For all statistical analyses, significance was accepted at the 95% confidence level, and all analyses were two-tailed. Statistical differences were defined as *P<0.05, **P<0.01, and differences with P<0.05 were considered significant.

A cream gel formulation for anti-adhesion applications inhibits postoperative adhesion as demonstrated herein in multiple rat models, including abdominal wall defect-cecum abrasion adhesion model and repeated-injury adhesion model.

Unlike current anti-adhesive films or hydrogels replying on suturing or curing to be immobilized on the traumatized surface, the cream gel formulation can be immediately immobilized on any irregular surface through convenient topical application without waiting for a curing step to occur, due to its injectable yet malleable and self-supporting properties. For instance, the cream gel can be conveniently injected through a syringe or a needle depending on the surgical need to fully cover a wound surface without performing hemostasis, and then immediately remain on the injured surface even when the surface was inverted or rotated.

In this example, preparation of a biodegradable cross-linked zwitterionic polymer cream gel is described. Carboxybetaine acrylamide (CBAA) monomer was crosslinked with a crosslinker containing disulfide bonds, N,N′-bis(acryloyl)cystamine (BAC). After complete equilibration in phosphate-buffered saline (PBS), the resulting bulk zwitterionic polymer gel was processed into a zwitterionic polymer cream gel by grinding and repeatedly extruding the material through different gauge needles. The resulting zwitterionic polymer cream gel has a mean particle diameter of 20-50 m and can be easily injected through a 26-gauge needle.

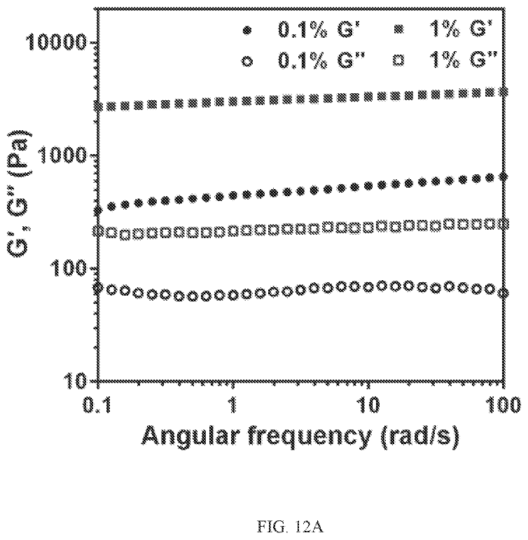

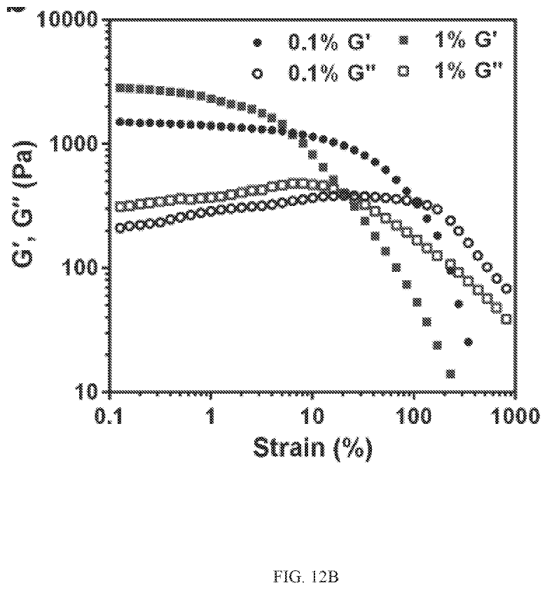

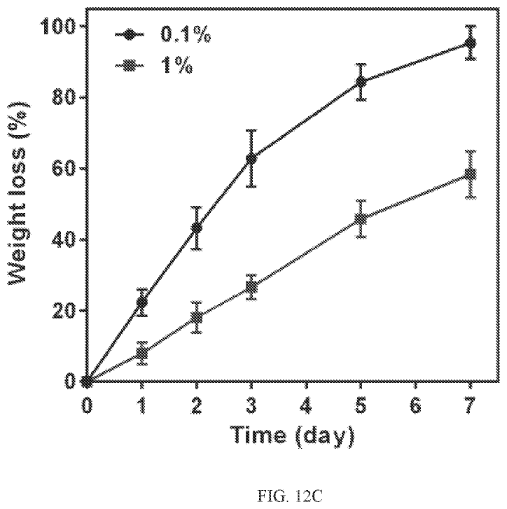

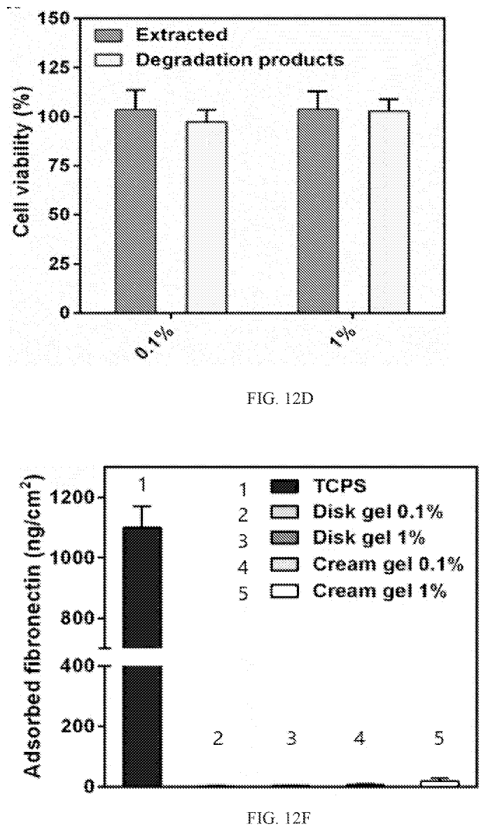

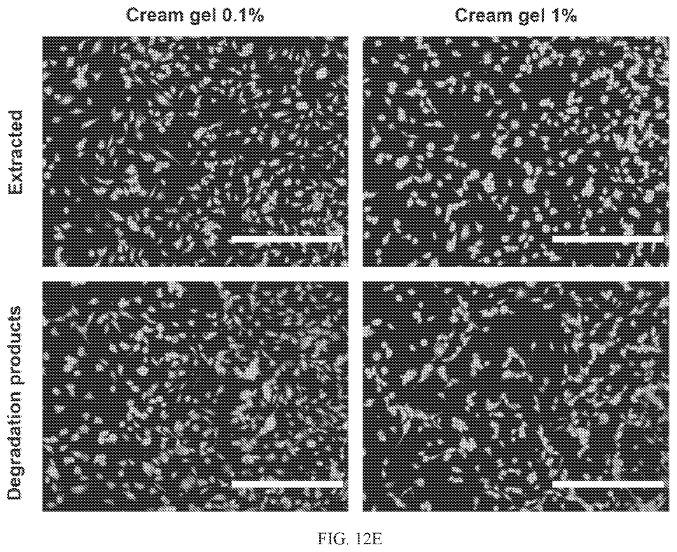

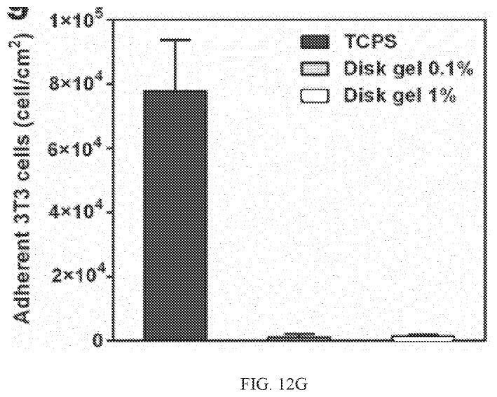

The viscoelastic properties of the produced biodegradable cross-linked zwitterionic polymer cream gels were studied using rheological tests. Frequency-dependent oscillatory sweeps in the linear viscoelastic regime (under 1% strain) showed that the storage modulus (G′) was dominant over the loss modulus (G″) over the full angular frequency range (0.1-100 rad/s), see FIG. 12A. This indicated that zwitterionic polymer cream gels with different crosslinker content (0.1% or 1% BAC) maintained a solid-like behavior over the entire frequency range. In a strain-dependent oscillatory rheological measurement, the zwitterionic polymer cream gels maintained the solid-like behavior at the low strain range (0.1-10%) with G′ higher than G″, but adopt more viscous behavior at high strain region where G′ became lower than G″, see FIG. 12B. It should be noted that the highest strain experienced by tissues in the body has been reported to be less than 10%.^{[38]}

The biodegradability of the biodegradable cross-linked zwitterionic polymer cream gels was examined in 10 mL of PBS containing 20 μM GSH (mimicking the concentration in bloodstream and body fluids^{[39, 40]}) at 37° C. All of the biodegradable cross-linked zwitterionic polymer cream gels were found to gradually degrade over time as reflected by the weight loss, see FIG. 12C. of Biodegradable cross-linked zwitterionic polymer cream gel with 0.1% BAC crosslinker had more than 60% degraded on day 3 and almost totally degraded on day 7. Biodegradable cross-linked zwitterionic polymer cream gel with 1% BAC showed a slower degradation rate, with less than 60% degraded on day 7.

Cytotoxicity of both an extract of biodegradable cross-linked zwitterionic polymer cream gel and the degradation products (zwitterionic polymer cream gel degraded in DMEM with 20 mM GSH for 48 h at 37° C.) was tested using an in vitro cell viability test and live/dead imaging. Both the extract and degradation products exhibited no significant cytotoxicity on the 3T3 cells regardless of the BAC content, see FIG. 12D, which was also confirmed through live/dead staining, see FIG. 12E.

It has been well recognized that fibronectin adsorption and fibroblast adhesion lead to a connection between damaged intra-abdominal surfaces and subsequent adhesion development. An in vitro study of this example showed, using a BCA protein quantification assay, that little fibronectin was able to adhere on the surface of disk gels or zwitterionic polymer cream gels, see FIG. 12F. By contrast, a large amount of fibronectin, >1000 ng/cm², was found to adhere on the tissue culture plate surface (TCPS). In a further experiment, fibroblasts (3T3 cells) were seeded on the surfaces of disk gels and zwitterionic polymer cream gels followed by cell staining and fluorescent microscopy. Few spherical cells were observed on the disk gel surface, whereas a large number of 3T3 cells settled and spread on the TCPS, see FIG. 12G, 12H, and FIG. 13.

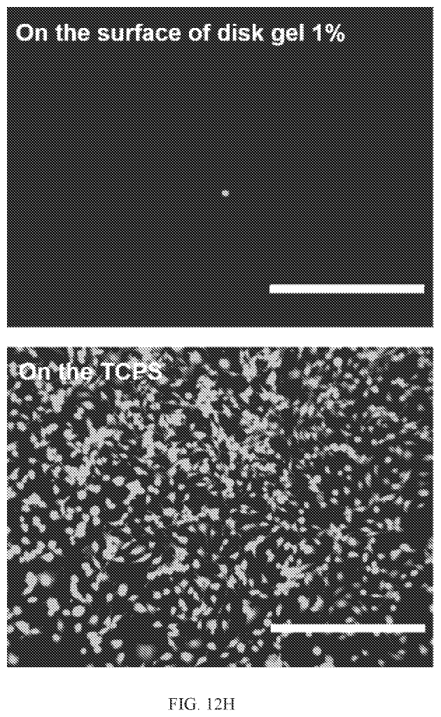

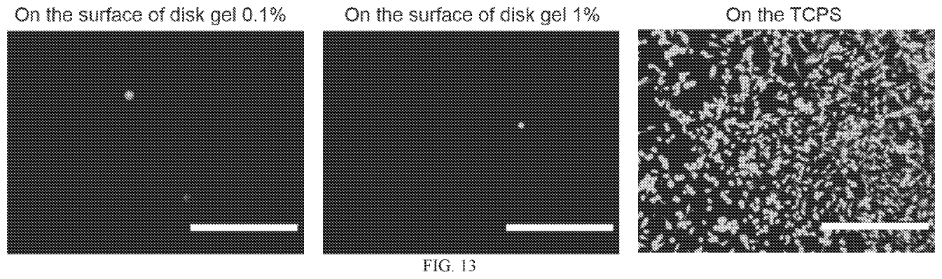

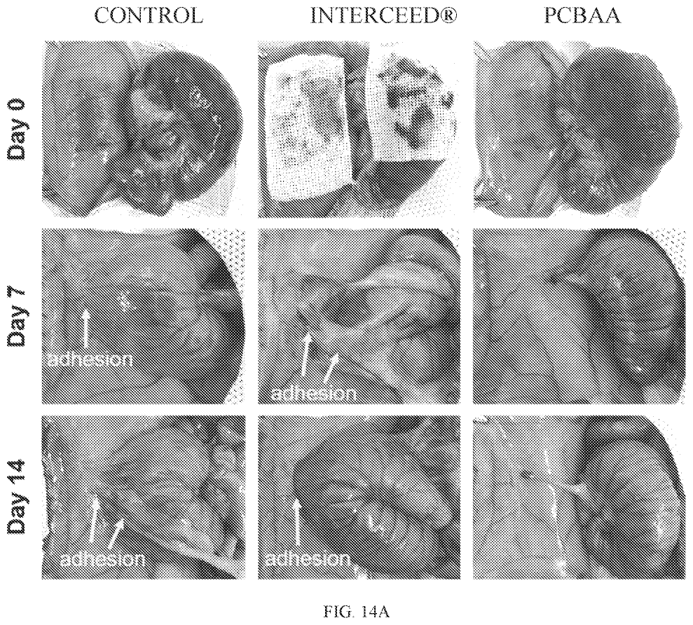

The anti-adhesion efficacy of the zwitterionic polymer cream gel was evaluated in a rat sidewall defect-cecum abrasion model. Defects on the cecum and the abdominal wall were created by abrasion and excision, respectively, followed by the application of 2 mL of zwitterionic polymer cream gel on both traumatized surfaces, see FIG. 14A. On day 7 and 14 post-surgery, the peritoneum was opened and examined and the level of adhesion, if any, was scored. The group without any anti-adhesion treatment showed severe abdominal adhesions on day 7 and 14 post-surgery, see FIGS. 14A and 14B. Treatment with a commercial Interceed® film (2×2 cm) appeared to lower the adhesion score compared with the untreated group but significant adhesions were still present. Zwitterionic polymer cream gel treatment, by contrast, provided nearly a complete protection: on day 7, 6 out of 6 animals showed no observable adhesion (score 0); on day 14, 5 out of 6 animals showed no observable adhesion and 1 out of 6 animals had a minor adhesion (score 1). The change of body weight 7 or 14 days post-surgery did not vary significantly among these groups (p >0.05), see FIG. 14C. Tissues from injured sites were collected for histological analysis at day 7 and 14 post-surgery. Results from Hematoxylin and Eosin, and Masson trichrome staining supported the observative scoring: connective/adhesive regions can be easily identified for the untreated and film treated groups (the deposited collagen in the adhesion site was stained in blue while muscle showed red in Masson trichrome staining), but can hardly be located in zwitterionic polymer cream gel treated group since no adhesion developed at all (histology for injured abdominal wall and cecum was performed separately), see FIGS. 14D and 14E. It should also be noted that the biodegradable zwitterionic polymer cream gel was still present on the injured sites, such as the abdominal wall, on day 7 but fully disappeared by day 14, as confirmed by both autopsy and histological examination. Tissue sections from major organs collected on day 7 and 14 indicated no damage or inflammatory reactions in the zwitterionic polymer cream gel treated group compared with healthy, untreated, tissues, see FIG. 15. These results overall indicate that the biodegradable zwitterionic polymer cream gel was able to completely and reliably prevent adhesion in the abdominal wall defect-cecum abrasion adhesion model, and was effectively cleared over time without eliciting toxicity.

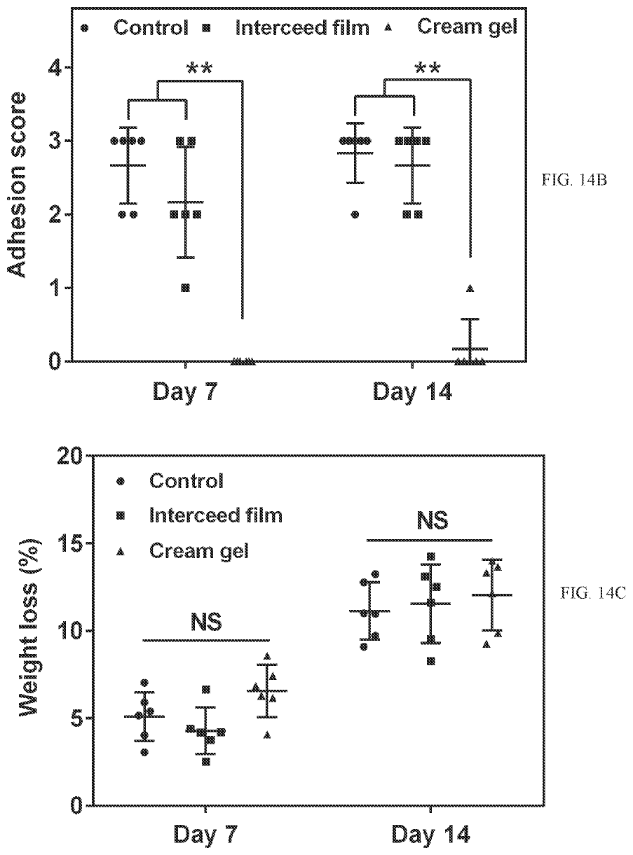

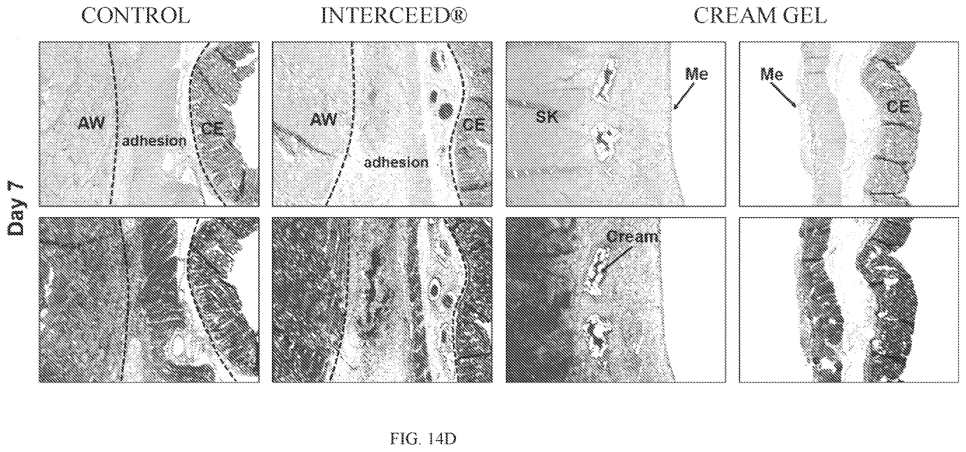

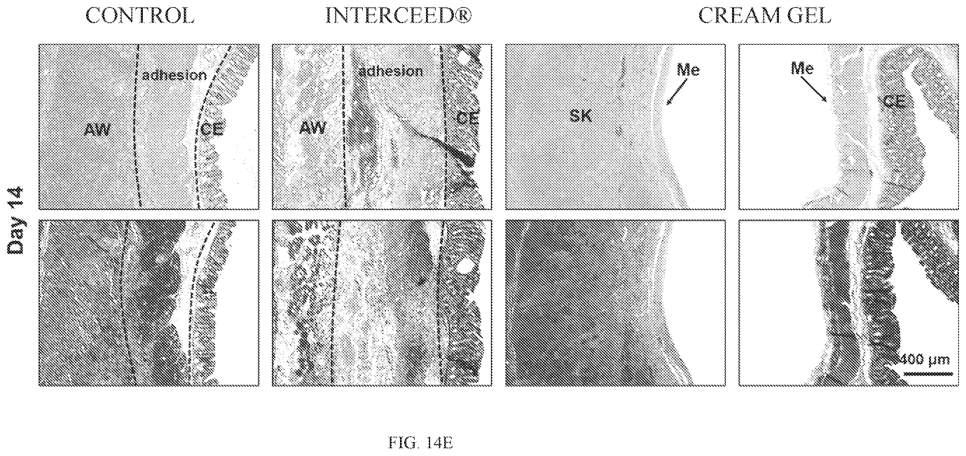

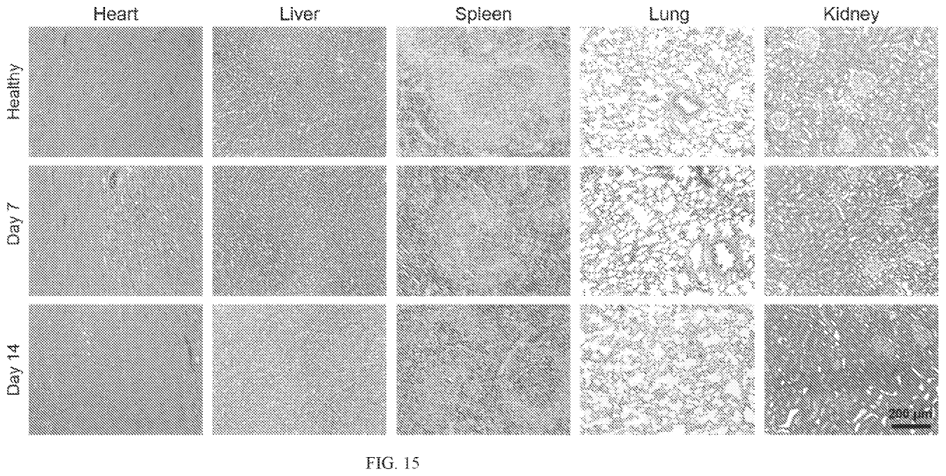

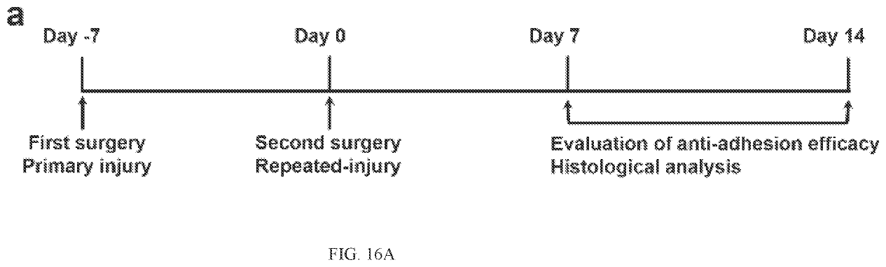

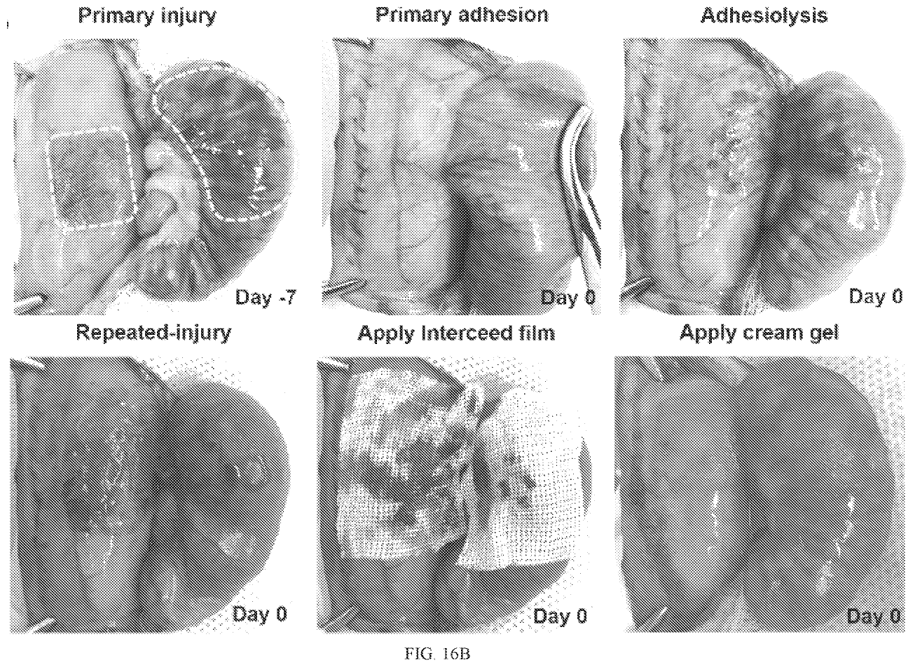

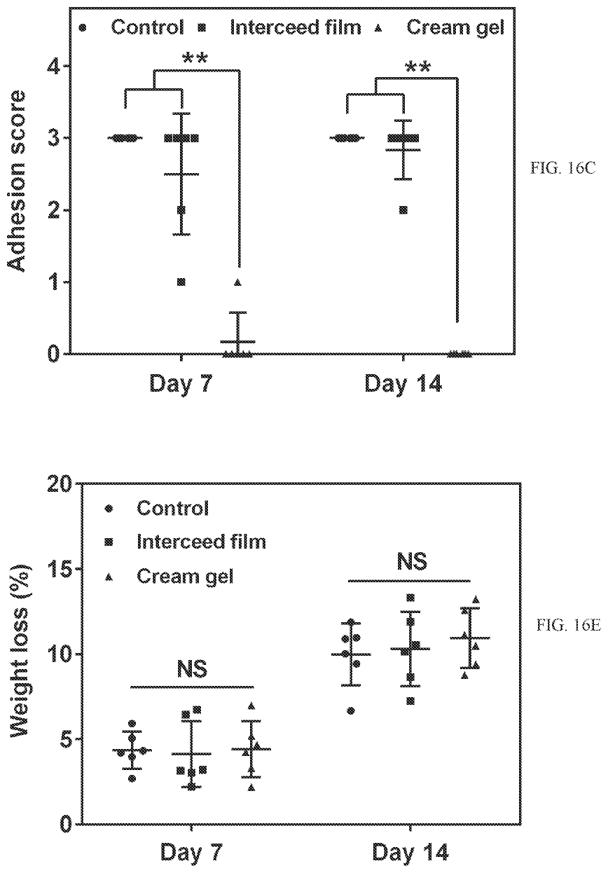

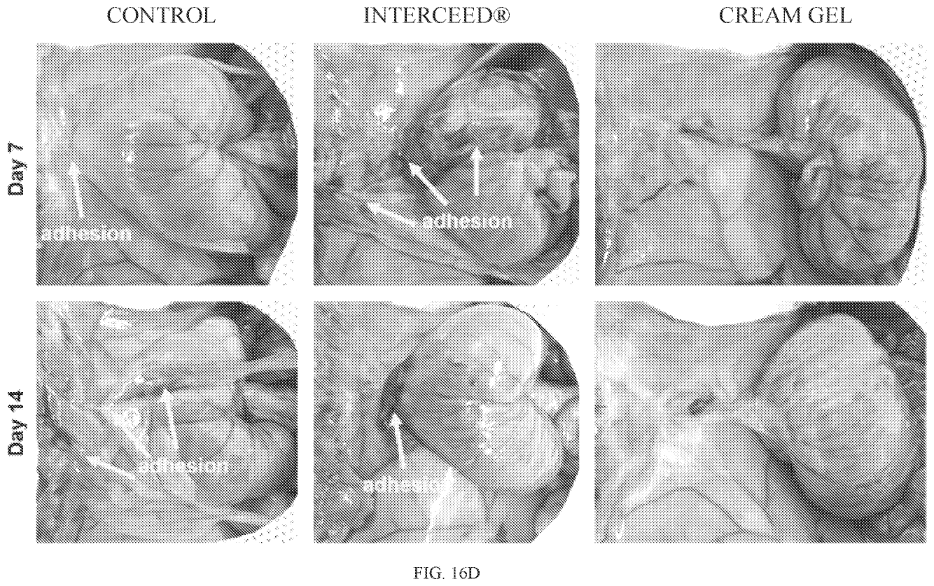

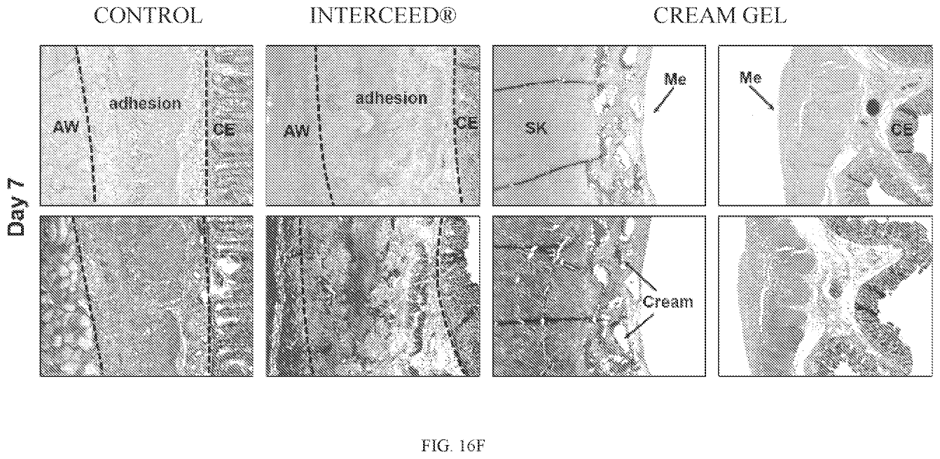

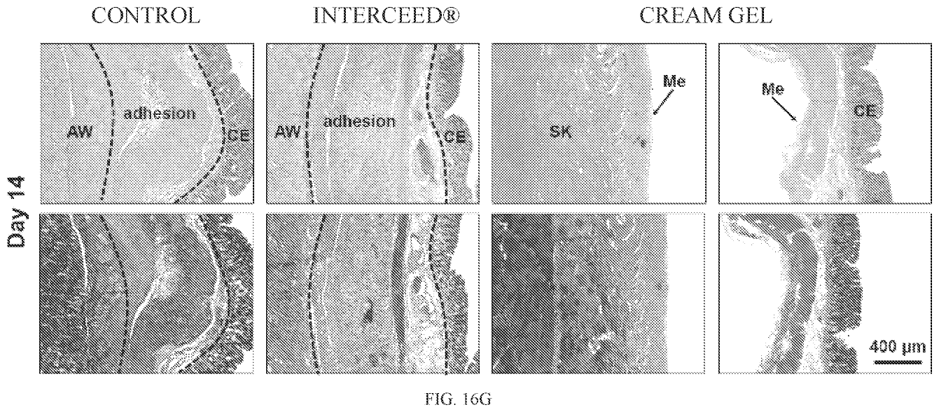

Anti-adhesion efficacy of the zwitterionic polymer cream gel was evaluated in a rigorous rat repeated-injury adhesion model. This model mimics when a recurrent adhesion was developed after conducting adhesiolysis. Adhesolysis was used to treat a primary adhesion but created more severe trauma and adhesion. To establish the repeated-injury adhesion model, a first abdominal wall and cecum injury was created without any anti-adhesion treatment. 7 days later, the primary adhesion was developed, adhesiolysis was conducted, and a second injury was created. This was followed by zwitterionic polymer cream gel treatment, film treatment, or no treatment, in separate animal groups, see FIGS. 16A, 16B. Compared with the primary adhesion model, see FIGS. 14A-14E, this recurrent model presented a much more severe adhesion when no treatment applied, i.e. all animals had a score 3 adhesion on both day 7 and 14 post surgery, see FIGS. 16C, 16D. Commercial film treatment only slightly relieved the recurrent adhesion. However, zwitterionic polymer cream gel treatment remarkably achieved a nearly complete prevention of recurrent adhesion: on day 7, 5 out of 6 animals treated with the zwitterionic polymer cream gel showed no observable adhesion (score 0) and 1 out of 6 animals so treated had a minor adhesion (score 1); on day 14, 6 out of 6 animals treated with the zwitterionic polymer cream gel had no observable adhesion. The change of body weight 7 or 14 days post-surgery also not differed significantly among groups (p >0.05), see FIG. 16E. Histological results for day 7 and 14 were consistent with the scoring results, supporting the reliable anti-adhesion efficacy of the zwitterionic polymer cream gel, see FIGS. 16F and 16G. The biodegradable zwitterionic polymer cream gel appeared to be similarly cleared as in the primary adhesion model, with gel particles present on the injured abdominal wall on day 7 but completely gone on day 14, as shown by both autopsy and histology. Further examination on day 30 post surgery showed that the zwitterionic polymer cream gel treated tissue recovered from the severe injury to a condition as good as a healthy, untreated, tissue. These results further support the efficacy of the zwitterionic polymer cream gel in completely and reliably preventing the more rigorous recurrent adhesion in the rat repeated-injury adhesion model.

These results show that a zwitterionic polymer-based cream gel was able to completely and reliably prevent a primary and a more severe recurrent adhesion in rat models.

Zwitterionic PCBAA polymer prevented postoperative adhesion in a pig abdominal wall defect-cecum abrasion adhesion model (validation in a large animal model).

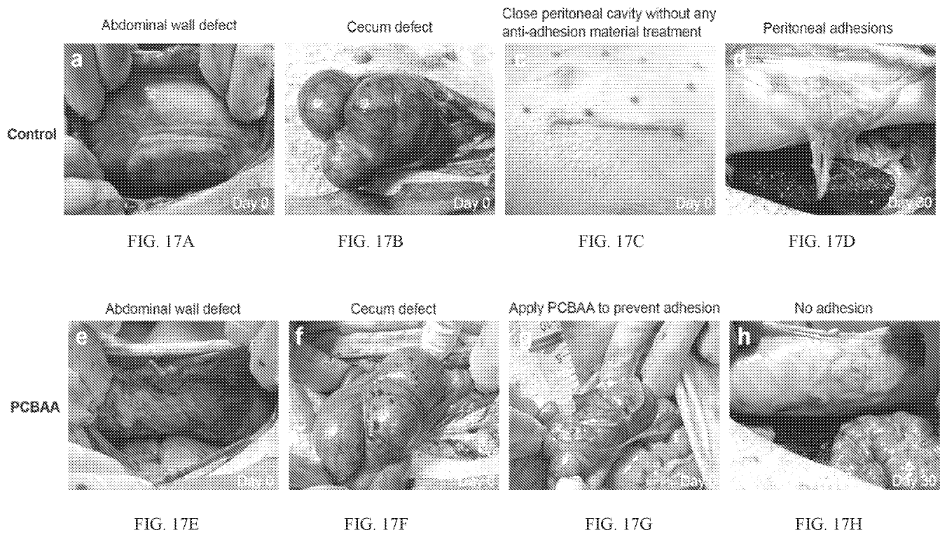

Two Ellegaard Gottingen Minipigs (male, 30-33 kg) were used for anti-adhesion evaluation of zwitterionic polymer PCBAA. Pigs received laparotomy with the serosa of the cecum wall abraded using a sterile Bovie scratch pad until the serosa was damaged and hemorrhaging but not perforated to create a 6×6-cm defect (FIGS. 17A-17H). Then a 6×6-cm area of the parietal peritoneum in the right hypogastrium opposed to the cecum was excised with scissors. One pig received 20 mL zwitterionic polymer PCBAA treatment on the injured sites followed by peritoneum and skin closure. The other pig served the untreated control. Within 3 days after the surgery, untreated pig suffered severe abdominal pain and required analgesic interventions at 3 times regular doses. Zwitterionic polymer PCBAA treated pig behaved normally without any interventions. On day 30, the pigs were sacrificed. Severe peritoneal adhesions developed on the untreated pigs, while the zwitterionic polymer PCBAA-treated pig was free from any observable adhesion (FIGS. 17D, 17H). This result further validates the efficacy of zwitterionic polymer PCBAA in a pig model.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1.-14. (canceled)

15. A packaged dosage unit of an implantable anti-adhesion zwitterionic composition, comprising: an aqueous liquid and a zwitterionic polymer or copolymer, the zwitterionic polymer or copolymer comprising a plurality of repeating units, where each repeating unit has structural formula (VI), (VIII), or (X): 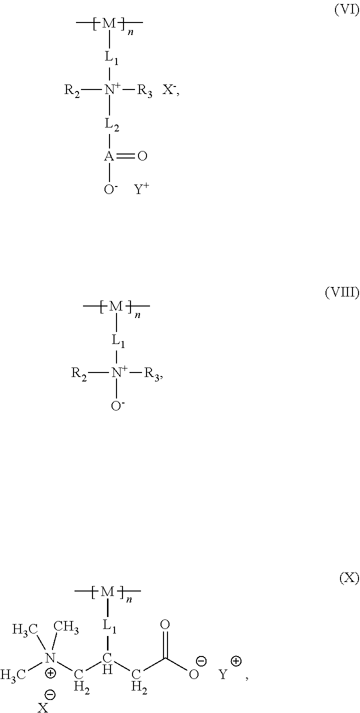
where M is a monomeric repeating unit, n is an integer from 1 to about 100,000, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

16. The packaged dosage unit of an implantable anti-adhesion zwitterionic composition of claim 15, wherein the zwitterionic polymer and/or copolymer comprises a polymerization reaction product of a zwitterionic monomer having structural formula (I), (III), or (V): 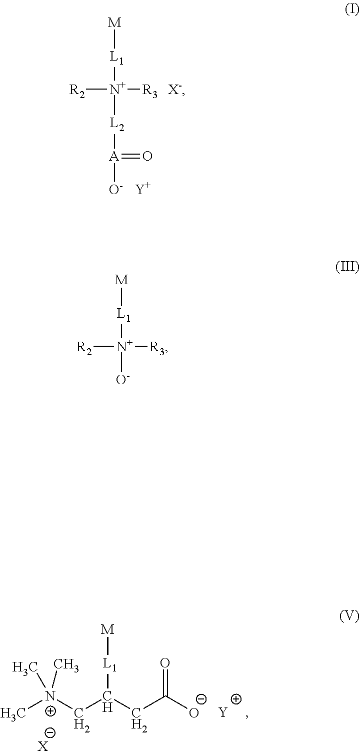
where M is a monomeric repeating unit, where R₂, and R₃ are each independently selected from hydrogen, alkyl, and aryl groups, L₁ is a linker that covalently couples a cationic center to a polymer backbone; L₂, if present, is a linker that covalently couples the cationic center to an anionic group; A(═)O⁻ if present, is an anionic group, X⁻, if present, is a counter ion associated with the cationic center, and Y+, if present, is a metal ion, an ammonium ion, or an organic ion.

17. The packaged dosage unit of an implantable anti-adhesion zwitterionic composition of claim 15, wherein the zwitterionic polymer or copolymer comprises a plurality of repeating units, where each repeating unit has structural formula: 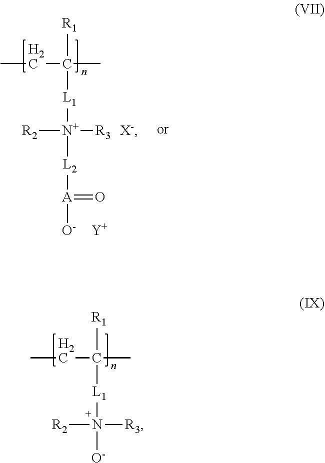
wherein R₁, R₂, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻ if present, is an anionic group, X⁻, if present, is a counter ion associated with the cationic center, and Y+, if present, is a metal ion, an ammonium ion, or an organic ion. n is an integer in the range of 2 to about 100,000.

18. The packaged dosage unit of an implantable anti-adhesion zwitterionic composition of claim 15, wherein the zwitterionic polymer and/or copolymer comprises a polymerization reaction product of a zwitterionic monomer having structural formula: 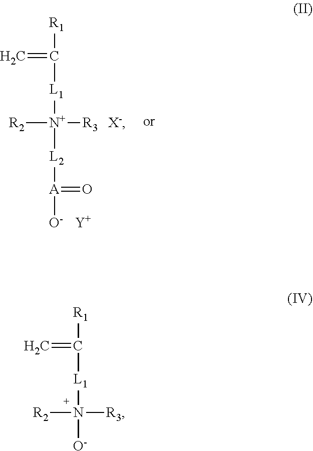
where R₁, R₂, and R₃ are each independently selected from: hydrogen, alkyl, and aryl groups; L₁ is a linker that covalently couples a cationic center to a polymer backbone; L₂, if present, is a linker that covalently couples the cationic center to an anionic group; A(═O)O⁻, if present, is an anionic group, X⁻, if present, is a counter ion associated with the cationic center, and Y+, if present, is a metal ion, an ammonium ion, or an organic ion.

19. The packaged dosage unit of an implantable anti-adhesion zwitterionic composition of claim 15, further comprising a crosslinker covalently bound to at least two of the monomeric repeating units.

20. The packaged dosage unit of an implantable anti-adhesion zwitterionic composition of claim 15, wherein the crosslinker is present in an amount in the range of 0.001 wt % to 50 wt %, 0.001 wt % to 20 wt %, 0.01 wt % to 10 wt %, 0.01 wt % to 5 wt %, 0.01 wt % to 1 wt %, 0.1 wt % to 1 wt %, relative to n monomeric repeating units

21. The packaged dosage unit of an implantable anti-adhesion zwitterionic composition of claim 15, further comprising a polymerization reaction product of a zwitterionic copolymer having reactive groups and comprising the structural formula (XI): 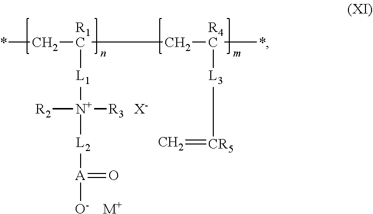
where R₁, R₂, R₃, R₄, and R₅ are each independently selected from hydrogen, alkyl, and aryl groups; L₁ is a linker that covalently couples a cationic center to the polymer backbone; L₂ is a linker that covalently couples the cationic center to an anionic group; A(═O)O⁻ is the anionic group; A is C, S, SO, P, or PO; X⁻ is the counter ion associated with the cationic center; and M⁺ is a metal ion, an ammonium ion, or an organic ion; L₃ is a linker that covalently couples a double bond to a polymer backbone, n is an integer in the range of 2 to about 100,000, m is a positive non-zero number; and m/n is in the range of 0.1%-10000%.

22. The packaged dosage unit of an implantable anti-adhesion zwitterionic composition of claim 15, wherein M is a repeating unit of a polymer selected from the group consisting of: polyester, polyamide, poly(amino acid), polyimide, polycarbonate, polysiloxane, polyurethane, polyphosphazene, acrylic polymer, amino resin, epoxy resin, phenolic resin, and alkyd resin.

23. The packaged dosage unit of an implantable anti-adhesion zwitterionic composition of claim 15, wherein A is C, S, SO, P, or PO.

24. The packaged dosage unit of an implantable anti-adhesion zwitterionic composition of claim 15, further comprising a polymerization reaction product of a zwitterionic copolymer having reactive groups and comprising the structural formula (XII): 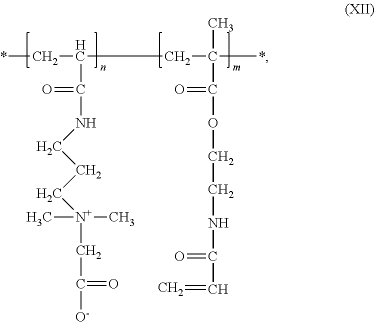
(XII), where n is an integer in the range of 2 to about 100,000, m is a positive non-zero number; and m/n is in the range of 0.1% to 10000%.

25. The packaged dosage unit of an implantable anti-adhesion zwitterionic composition of claim 15, wherein m/n is in the range of: 0.2% to 10%, 0.25% to 10%, 0.5% to 10%, 0.75% to 10%, 1% to 10%, 2% to 10%, 3% to 10%, 4% to 10%, 5% to 10%, 6% to 10%, 7% to 10%, 8% to 10%, 9% to 10%, 0.2% to 50%, 0.25% to 50%, 0.5% to 50%, 0.75% to 50%, 1% to 50%, 2% to 50%, 3% to 50%, 4% to 50%, 5% to 50%, 6% to 50%, 7% to 50%, 8% to 50%, 9% to 50%, 10% to 50%, 15% to 50% or 20% to 50%.

26. The packaged dosage unit of an implantable anti-adhesion zwitterionic composition of claim 15, comprising nanoparticles and/or microparticles of zwitterionic hydrogel.

27.-29. (canceled)

30. The packaged dosage unit of an implantable anti-adhesion zwitterionic composition of claim 26, wherein the nanoparticles and/or microparticles of zwitterionic hydrogel are present in an amount in the range of: 0.01 wt % to 40 wt %, 0.1 wt % to 40 wt %, 1 wt % to 30 wt %, 1 wt % to 20 wt %, 2 wt % to 10 wt %, 2 wt % to 6 wt %, relative to the aqueous liquid.

31. (canceled)

32. The packaged dosage unit of an implantable anti-adhesion zwitterionic composition of claim 15, wherein the zwitterionic polymer is, consists essentially of, or comprises, PCBAA having the structural formula: 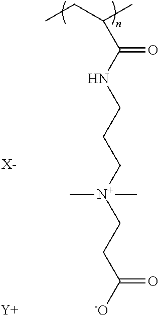
where n is an integer in the range of 2 to about 100,000, X⁻, if present, is a counter ion associated with the cationic center, and Y+, if present, is a metal ion, an ammonium ion, or an organic ion.

33.-49. (canceled)

50. A method of inhibiting tissue adhesion in a subject in need thereof, comprising:
administering an effective amount of an implantable anti-adhesion zwitterionic composition included in a packaged dosage unit of claim 15 to a tissue surface of the subject in need thereof.

51. The method of claim 50, wherein the tissue surface is exposed by surgery.

52. The method of claim 50, wherein the implantable anti-adhesion zwitterionic composition requires no curing following administration.

53. The method of claim 50, wherein the implantable anti-adhesion zwitterionic composition undergoes no polymerization or crosslinking reaction following administration.

54. The method of claim 50, wherein the implantable anti-adhesion zwitterionic composition is flowable and/or malleable, and conforms to the tissue surface.

55. The method of claim 50, wherein the implantable anti-adhesion zwitterionic composition does not undergo further chemical reaction which leads to an increase of the MW of the composition.
